(19) United States
(12) Patent Application Publication
(10) Pub. No.: US 2014/0364899 A1
(43) Pub. Date: Dec. 11, 2014

(54) SYSTEMS AND METHODS FOR IMPROVED VESSEL ACCESS CLOSURE

(71) Applicant: PROMED, INC., Santa Clara, CA (US)

(72) Inventors: Richard GINN, Gilroy, CA (US); Hans VALENCIA, Santa Clara, CA (US)

(21) Appl. No.: 14/301,889

(22) Filed: Jun. 11, 2014

Related U.S. Application Data
(60) Provisional application No. 61/833,842, filed on Jun. 11, 2013.

(51) Int. Cl. A61B 17/00

(52) U.S. Cl. CPC A61B 17/0057; A61B 2017/00601
USPC 606/213

(57) ABSTRACT

Embodiments are described for closing vascular access ports, such as arteriotomies, which involve placement and deployment of an expandable device configured to prevent blood flow across a subject arteriotomy while also keeping disturbance of intravascular flow to a minimum. Suitable prostheses may comprise one or more frames constructed from lengths of flexible materials, such as shape memory alloys or polymers. Such frames may be coupled to sheetlike or tube-like structures configured to spread loads, minimize thrombosis which may be related to intravascular flow, and maintain hemostasis.

### CROSS-REFERENCE TO RELATED APPLICATIONS

The present application is a non-provisional of U.S. Patent Application Ser. No. 61/833,842, filed Jun. 11, 2013, the full disclosure of which is incorporated herein by reference.

### FIELD OF THE INVENTION

The present invention relates generally to closure of surgically created vascular access ports or holes, such as arteriotomies, and more specifically to closure technologies pertinent to relatively large surgically-created access defects.

### BACKGROUND

Minimally invasive diagnostic and interventional procedure prevalence in US and foreign hospitals continues to increase, as does the demand for certain procedures which involve placement of relatively large devices into targeted locations that are initially accessed through the vasculature. For example, percutaneous prosthetic heart valve placement and abdominal aortic aneurysm stent graft procedures that are not accomplished using one or more trans-thoracic or trans-abdominal access ports generally involve one or more femoral arteriotomies which may be large in size relative to conventional femoral arteriotomies, due, at least in part, to the size of devices utilized for such procedures. Subsequent to completion of the diagnostic or interventional aspects of such treatments, any associated arteriotomies generally must be closed. While there are existing technologies for closing defects created in veins and arteries due to diagnostic and/or interventional tool access, such as those available from St. Jude Medical, Inc., Abbott Laboratories, Inc., and Access Closure, Inc. under the tradenames Angio-Seal®, StarClose®, and Mynx®, respectively, none of these are well suited for closing relatively large defects—particularly not in the arterial environment wherein relatively high flow rate and pressure are complicating factors.

### BRIEF DESCRIPTION OF THE DRAWINGS

The details of the inventive subject matter, both as to its structure and operation, may be gleaned in part by study of the accompanying figures, in which like reference numerals refer to like parts. The components in the figures are not necessarily to scale, emphasis instead being placed upon illustrating principles. Moreover, all illustrations are intended to convey concepts, where relative sizes, shapes and other detailed attributes may be illustrated schematically rather than literally or precisely. Explicit or implicit description of subject matter in the written text of this document shall be a basis for claiming that subject matter, regardless of whether that subject matter is also depicted in the accompanying figures. Likewise, the explicit or implicit presence of any subject matter in a particular figure shall be a basis for claiming that subject matter, regardless of whether that subject matter is also depicted in the written text of this document. Aspects of the inventive subject matter are illustrated by way of example, and not by way of limitation, in the accompanying drawings wherein:

FIGS. 1A-1E illustrate aspects of an access closure device deployment wherein a collapsed device is passed through an introducer lumen to a targeted intravascular deployment position where it is deployed.

FIGS. 1F and 1G illustrate cross sectional views of two different embodiments of deployed closure device configurations.

FIGS. 1H-1J illustrate orthogonal views of two different embodiments of deployed closure device configurations.

FIGS. 1K and 1L illustrate cross sectional views of two different embodiments of deployed closure device configurations.

FIGS. 2A-2F illustrate aspects of an access closure device deployment wherein a two-portion collapsed device is passed through an introducer lumen to a targeted intravascular deployment position before expansion.

FIGS. 2G-2I illustrate side views and an orthogonal view, respectively, of a suitable device frame configuration.

FIGS. 3A-3D illustrate orthogonal views of a device embodiment that may be rolled up into a collapsed shape, and unrolled or unfurled to an expanded shape.

FIG. 4 illustrates one embodiment of a manual operational interface configuration.

FIG. 5 illustrates various aspects of an arteriotomy closure method in accordance with the present invention.

FIGS. 6A to 6Z-1 illustrate various aspects of arteriotomy closure configurations wherein a collapsed closure device may be controllably rotated relative to an elongate deployment member to prevent withdrawal of the closure device through the arteriotomy.

FIGS. 7A-7F illustrate various aspects of an arteriotomy closure device deployment wherein a collapsed closure device may be controllably rotated relative to an elongate deployment member to prevent withdrawal of the closure device through the arteriotomy.

FIG. 8A illustrates aspects of deployment steps using a configuration such as that illustrated in FIGS. 6A-6X, wherein a foot is pre-biased to flex once released from a restraining sheath.

FIG. 8B illustrates aspects of deployment steps using a configuration such as that illustrated in FIGS. 6A-6X, wherein a foot is flexed after release from a restraining sheath, subsequent to application of a flexing load.

FIGS. 9A-9E illustrate various aspects of arteriotomy closure configurations wherein a collapsed closure device may be controllably rotated relative to an elongate deployment member to prevent withdrawal of the closure device through the arteriotomy.

FIG. 10 illustrates aspects of deployment steps using a configuration such as that illustrated in FIGS. 9A-9E, wherein a foot is pre-biased to flex once released from a restraining sheath.

FIG. 11A illustrates aspects of a deployment sheath configured to assist an operator with positioning of related instrumentation adjacent an arteriotomy location.

FIGS. 11B-H illustrate various aspects of an example embodiment of a delivery system configured with bleed-back capability.

FIGS. 12A-12D illustrate aspects of a proximal deployment interface which may be utilized with closure device deployment configurations such as those depicted in FIGS. 9A-9E.

FIGS. 13A-13D illustrate various aspects of arteriotomy closure configurations wherein a collapsed closure device may be deployed as facilitated by a catch member configured to prevent withdrawal of the closure device after insertion through the arteriotomy.

FIGS. 14A-14B illustrate various aspects of arteriotomy closure configurations wherein a collapsed closure device may be deployed as facilitated by a catch member configured to prevent withdrawal of the closure device after insertion through the arteriotomy.

FIGS. 15A-15C illustrate three views of a closure device frame configuration including a catch member configured to aid in anchoring and prevent withdrawal after insertion through an arteriotomy and subsequent expansion in situ.

FIGS. 16A-16K illustrate various aspects of arteriotomy closure configurations wherein a collapsed closure device may be deployed as facilitated by a catch member configured to prevent withdrawal of the closure device after insertion through the arteriotomy.

FIG. 17 illustrates various aspects of a process flow for deploying an arteriotomy closure configuration wherein a collapsed closure device may be deployed as facilitated by a catch member configured to prevent withdrawal of the closure device after insertion through the arteriotomy.

FIGS. 18A-18C illustrate various aspects of a closure device configuration wherein each structure thereof may include a bioresorbable material, and wherein expansion from a collapsed state to an expanded state may include an unfurling transformation.

FIGS. 18D-18G illustrate various aspects of an inflatable closure device configuration wherein each structure thereof may include two-layer bioresorbable material, and wherein expansion from a collapsed state to an expanded state may include an unfurling transformation.

FIGS. 18H-18I illustrate a top-down view of an inflatable closure device in an unfurled configuration wherein each closure device includes a two layer bioresorbable material that dot or slot welded together.

FIGS. 18J-18M illustrate various aspects of a two-layer closure device configuration wherein each structure thereof may include a bioresorbable material, and wherein sutures are routed between the two-layer arrangement.

FIG. 19 illustrates various aspects of a process flow for deploying an arteriotomy closure configuration wherein a collapsed closure device may be deployed and expanded in an unfurling transformation.

FIGS. 20A-20K illustrate various aspects of arteriotomy closure configurations wherein a collapsed closure device may be deployed as facilitated by a catch member configured to prevent withdrawal of the closure device after insertion through the arteriotomy, and wherein a guidewire may be left in place to facilitate easy re-access to the endovascular structures near the arteriotomy.

FIG. 21 illustrates various aspects of a process flow for deploying an arteriotomy closure configuration wherein a guidewire may be left in place to facilitate easy re-access to the endovascular structures near the arteriotomy.

FIGS. 22A-22C illustrate various aspects of embodiments configured to employ an elongate guiding member such as a guidewire to facilitate ease of re-access after closure.

FIGS. 23A-23C illustrate various aspects of embodiments configured to employ a proximal catch member to effect a hole closure, and in the embodiments of FIGS. 23B and 23C, a distal catch member as well.

FIG. 24 illustrates various aspects of a process flow for deploying an arteriotomy closure configuration wherein a guidewire may be left in place to facilitate easy re-access to the endovascular structures near the arteriotomy.

FIG. 25 illustrates various aspects of a process flow for deploying an arteriotomy closure configuration wherein a guidewire may be left in place to facilitate easy re-access to the endovascular structures near the arteriotomy.

FIGS. 26A-B illustrate various aspects of an example embodiment of a delivery system for delivery of an arteriotomy closure device with a foot member.

FIGS. 26C-E illustrate various aspects of an example embodiment of a foot member coupled with an arteriotomy closure device.

FIGS. 26F-H illustrate various aspects of an example embodiment of a foot frame for use in the example foot member of FIGS. 26C-E.

FIG. 27 illustrates various aspects of an example embodiment of an arteriotomy closure system having a delivery system and introducer.

FIG. 28 illustrates various aspects of an example embodiment of an arteriotomy closure procedure.

FIGS. 29A-B illustrate various aspects of an alternative embodiment of an arteriotomy closure system, wherein the distal end of the delivery system has a soft tip.

FIGS. 30A-B illustrate a cross section and an elevated perspective view of an alternative embodiment of an arteriotomy closure system, wherein a hook protrudes from the distal end of the outer sheath to prevent inadvertent removal through the arteriotomy.

FIGS. 31A-31B illustrate a cross section of an alternative embodiment of an arteriotomy closure system, wherein a collapsible hook protrudes from the distal end of the outer sheath, capable of being drawn towards the outer sheath by manipulating a suture.

FIGS. 32A-32B illustrate alternative hook embodiments, wherein the hook is retracted into the outer sheath.

FIGS. 33A-33C illustrate a cross section of an outer sheath, wherein a collapsible hook protrudes from the distal end of the outer sheath through exit holes or apertures.

FIGS. 34A-34B illustrate a cross section of a closure device configured to be deployed prior to the contemplated surgical procedure.

FIGS. 35A-35B illustrates example embodiments of a hook wire for use in the example embodiments of FIGS. 36A-36D, 38A-38D, and 39A-39C.

FIGS. 36A-36D illustrate a cross section of an alternative embodiment of an arteriotomy closure system, wherein a collapsible hook is attached to a bottom portion of a distal end of an inner sheath and protrudes through the outer sheath, and is capable of being drawn within the outer sheath by manipulating the inner and outer sheath in relation to one another.

FIG. 37 illustrates an alternative example embodiment of a hook wire for use in the example embodiments of FIGS. 38A-38D.

FIGS. 38A-38D illustrate a cross section of an alternative embodiment of an arteriotomy closure system, wherein a collapsible hook is attached to a top portion of a distal end of an inner sheath and protrudes through the outer sheath, and is capable of being drawn within the outer sheath by manipulating the inner and outer sheath in relation to one another.

FIGS. 39A-39C illustrate a cross section of alternative embodiments of an arteriotomy closure system, wherein the collapsible hook is configured to be retained against the outer sheath to facilitate subsequent deployment of the hook.

### SUMMARY

Described herein are example embodiments of improved systems, devices, and methods for vessel access site closure with an expandable and implantable closure device deployed within the vessel. The closure device is implanted with a delivery system that generally includes an elongate member with a deflector coupled with the closure device and deployable from within an outer sheath. The deflector can be configured as a foot of the elongate member or as a component of a foot assembly. The delivery system can be configured with bleed-back capability to provide indication of current placement of the delivery system during the implantation procedure.

Other systems, methods, features and advantages of the subject matter described herein will be or will become apparent to one with skill in the art upon examination of the following figures and detailed description. It is intended that all such additional systems, methods, features and advantages be included within this description, be within the scope of the subject matter described herein, and be protected by the accompanying claims, or claimable at a later date. In no way should the contents of this summary section be used to further limit claims made on the subject matter described in this document.

### DETAILED DESCRIPTION

The breadth of the present subject matter is not to be limited to the examples provided and/or the subject specification, but rather only by the scope of claim language associated with this disclosure.

This application is related to U.S. application Ser. No. 13/205,528, filed Aug. 8, 2011 and U.S. Provisional Application Ser. Nos. 61/308,589, filed Feb. 26, 2010 and 61/353,561, filed Jun. 10, 2010, all of which are hereby incorporated by reference into the present application in their entirety for all purposes other than priority.

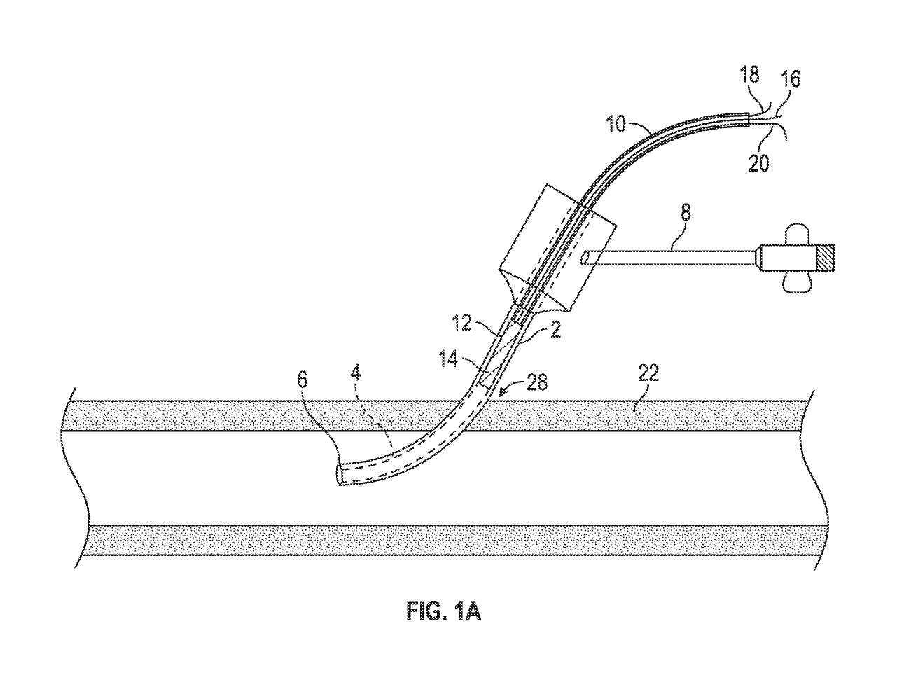

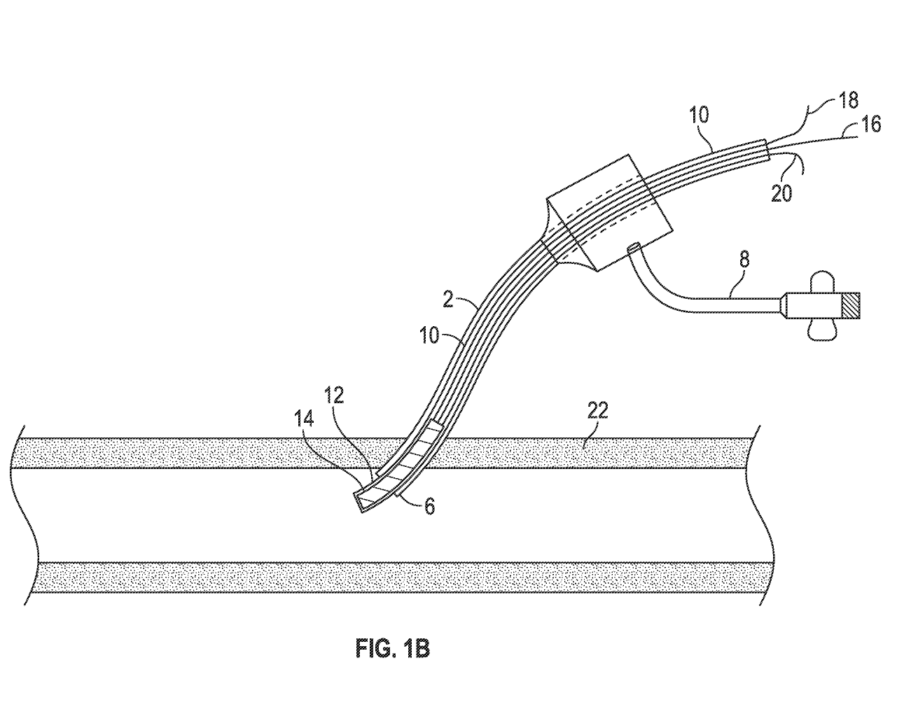

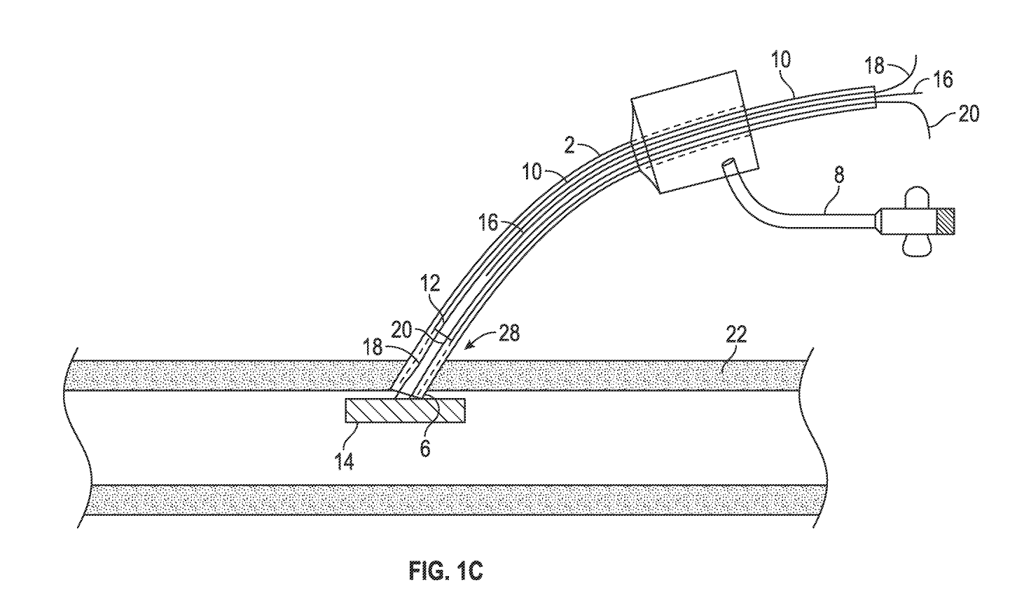

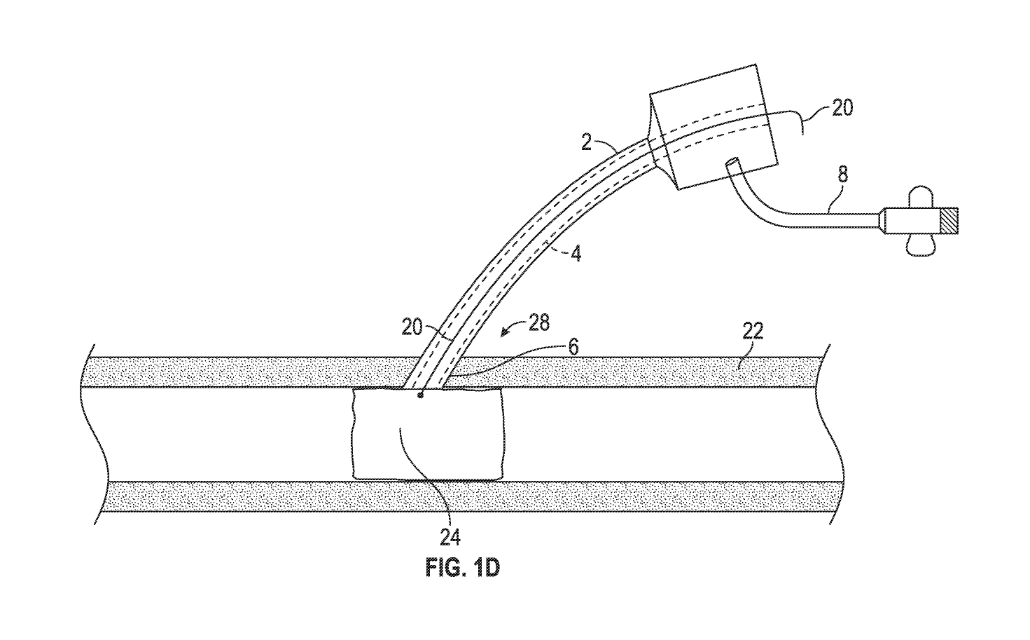

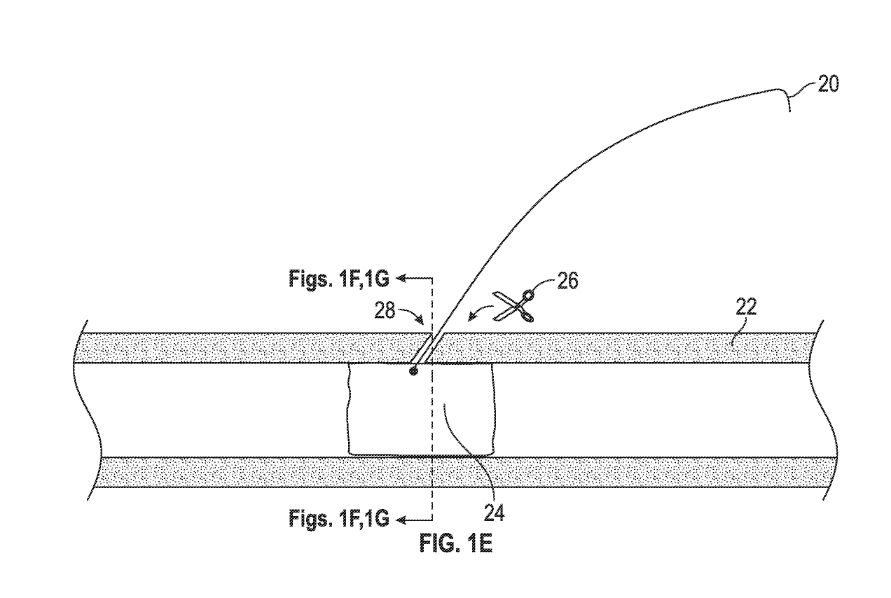

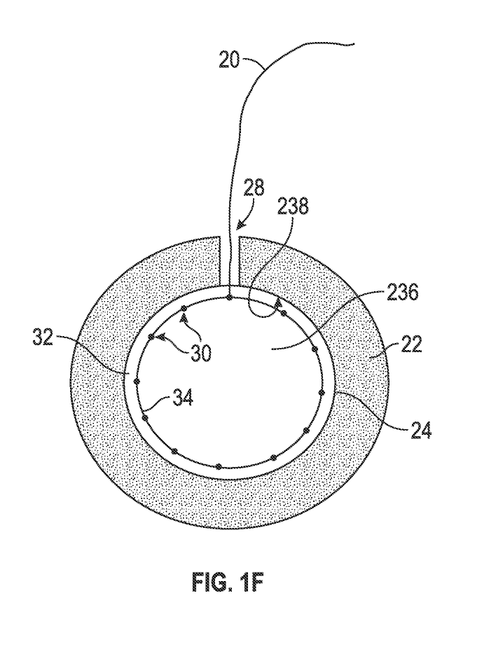

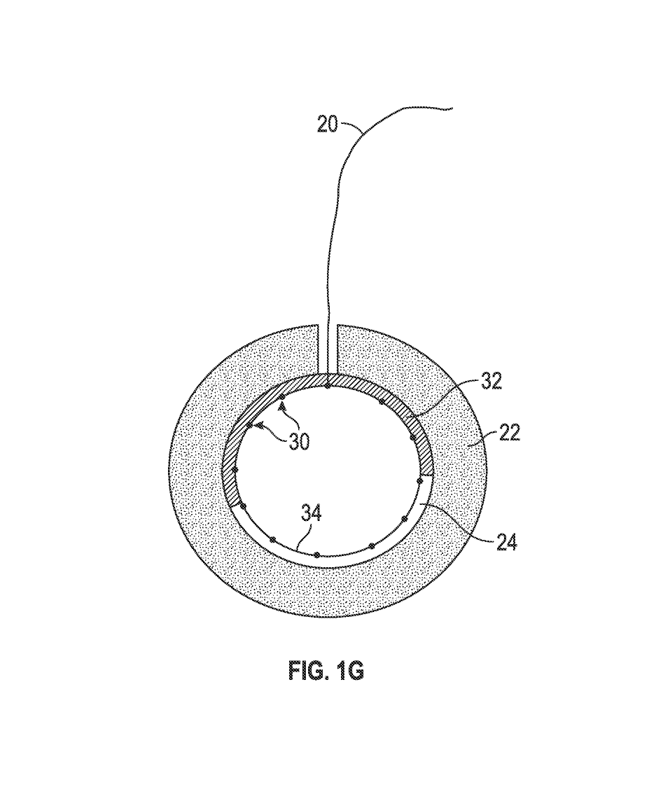

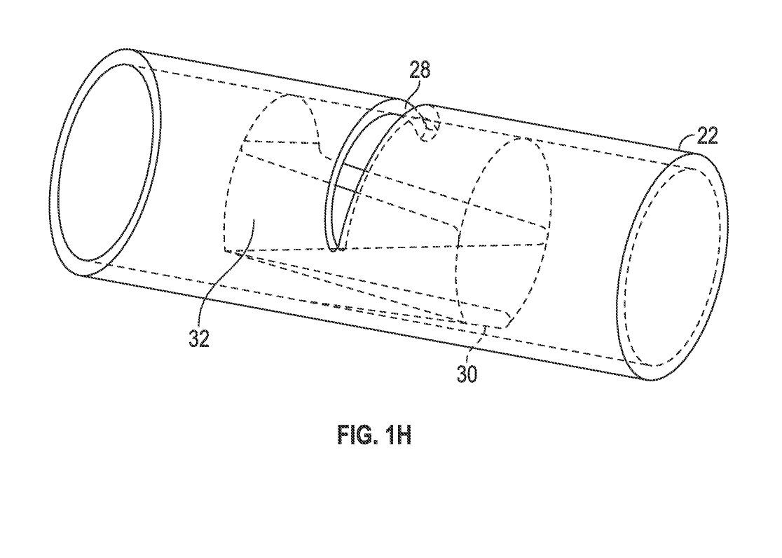

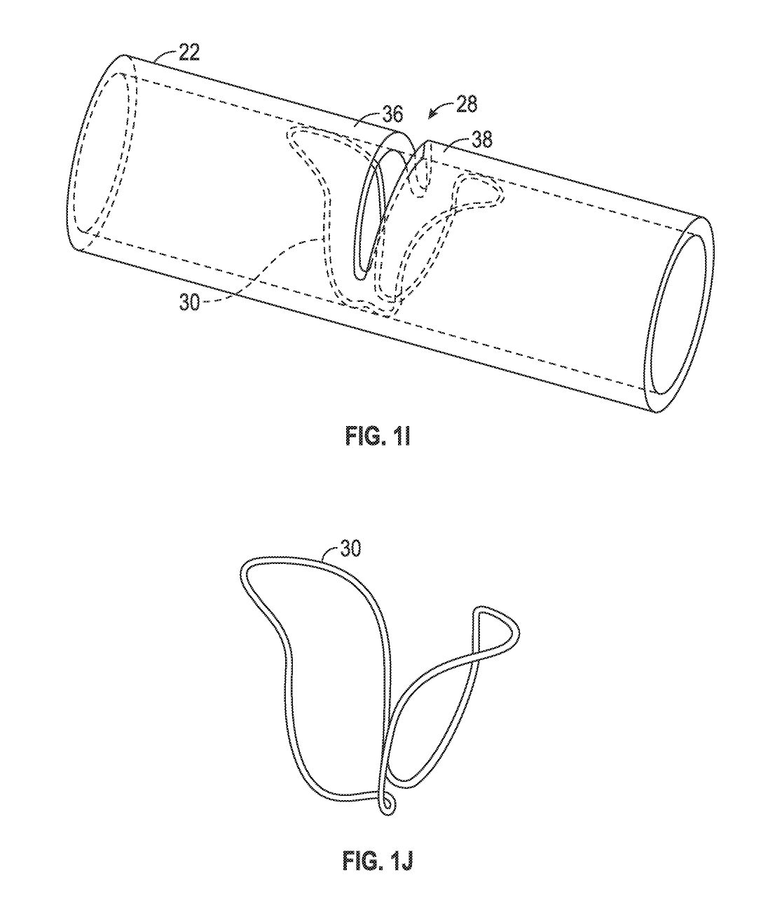

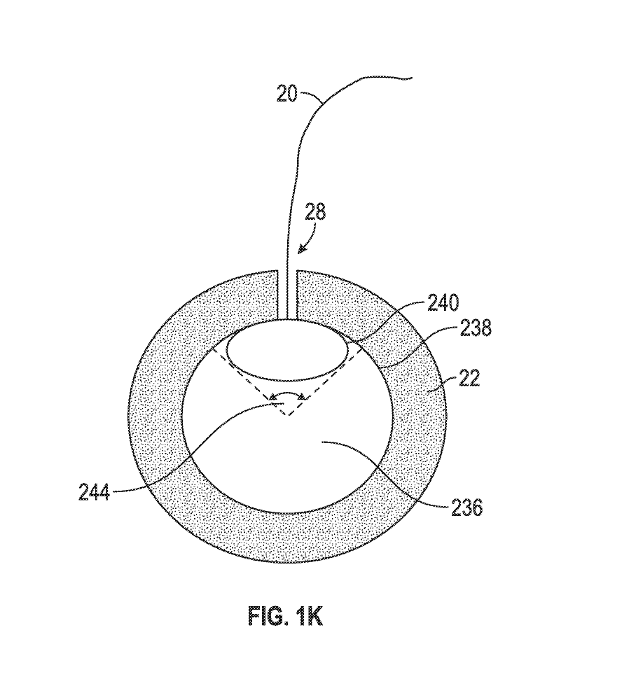

Referring to FIG. 1A, an introducer catheter (2) is shown with its distal tip (6) inserted across a hole formed in a blood vessel, such as an arteriotomy (28), which has been created in a blood vessel such as the femoral artery (22) to provide transvascular access for a procedure such as a percutaneous heart valve installation. The hole or arteriotomy (28) may have a diameter as large as 14 French or larger. In FIG. 1A, the valve deployment related tooling has been removed, and hemostasis through the lumen (4) defined through the introducer (2) may be controlled, for example, with valves integrated into the introducer or positive flush from an associated flush assembly (8). The embodiment depicted in FIG. 1A shows a closure device assembly being inserted through the introducer to facilitate execution of a controlled arteriotomy closure, the device assembly generally including a collapsed closure device (14), an elongate deployment member (10) having a distal portion (12) configured to removably accommodate the collapsed device (14), an insertion/retraction member (16) removably coupled with the device (14), a deployment tension member (18) configured to cause the collapsed device (14) be expandable to a deployed or expanded shape, and an attachment tension member (20) configured to pull the device proximally toward the operator of the assembly. In one embodiment the closure device (14) may be selected to span and close an arteriotomy having a diameter as large as 14 French or larger. The elongate deployment member preferably includes a flexible material construct, such as a polymer extrusion. The deployment and attachment tension members preferably include relatively small diameter sutures, wires, or tensile load bearing lines made from polymers and/or metals, such as polyethylene, polyethylene terepthalate, stainless steel, titanium, Nitinol, and the like. An insertion/retraction member (16) preferably is capable of not only withstanding tensile loads, but also relatively low-level compressive loads, as in a scenario wherein such structure is utilized to push a device (14) distally. Suitable materials for an insertion/retraction member (16) include the polymers and metals mentioned above in reference to the tension members (18, 20; in construction, given the desirable compressive functionality in addition to tensile, the insertion/retraction member generally will be stiffer, and potentially larger in diameter, as compared with such tension members (18, 20). The collapsed closure device (14) may include a plurality of flexible structural frame elements coupled together to form a collapsible and expandable member having an outer shape that is substantially cylindrical in both collapsed form and expanded form, and defining a lumen through the cylindrical expanded form, with generally no lumen defined through the generally cylindrical collapsed form. Further description of suitable closure device (14) details is featured below.

Referring to FIG. 1B, the delivery member (10) has been inserted farther through the introducer (2), and the distal portion (12) of the delivery member (10), with the collapsed device (14) confined therein, is being positioned past the distal tip of the introducer (6) and into the blood vessel (22).

Referring to FIG. 1C, with insertion of the insertion/retraction member (16) relative to the delivery member (10), the collapsed device (14) may be pushed out of the confining distal portion (12) of the delivery member (10) and into the free bloodstream space of the vessel (22). The attachment tension member (20) may then be tensioned to maintain the collapsed device in a position adjacent to the distal tip (6) of the introducer (2) as the delivery member (10) is retracted relative to the introducer (2), as shown in FIG. 1C.

Referring to FIG. 1D, with tensioning of the deployment tension member (element 18 in FIGS. 1A-1C), the device may be allowed to expand to an expanded shape (element 24 refers to the expanded form of the previously collapsed closure device, element 14) preferably substantially occupying the entire cross section of the blood vessel (22) and spanning well beyond the diameter of the arteriotomy (28) with space on either side to prevent exit of the closure device (24) through the arteriotomy (28) and achieve arterial closure. The deployment tension member (18) may include an elongate cable, suture, string, or the like configured to occupy very little cross sectional space, and to be able to withstand tensile loading sufficient, for example, to untie a knot configured to maintain the device in a collapsed configuration. One suitable tying or knot configuration includes what is known as a “highwayman's hitch” tied around the collapsed device (element 14 in FIGS. 1A-1C); with such a configuration, a controlled tensile pull on the deployment tension member from an operator at the proximal end of the instrument assembly causes the highwayman's hitch to untie, allowing the device to expand. Expansion from a collapsed state to an expanded state may be accomplished using a self-expanding device structure configured to expand itself to a final expanded shape, or an expandable shape configured to be expanded from a collapsed state to an expanded shape with the assistance of a balloon or other expansion device which may be placed through the device. In another embodiment, an expansion device including an expandable balloon or other expandable member configured to controllably expand based at least in part upon thermal energy, electric energy, shape memory, and/or hydrophilic expansion may be utilized to complete expansion of a device configuration which at least partially expands on its own, but which may require assistance to expand fully. As shown in FIG. 1D, the expanded closure device (24) remains coupled to the attachment tension member (20), and the introducer may remain located adjacent to the expanded device (24), holding the arteriotomy (28) open and providing a conduit for the attachment tension member to be used to make small adjustments in the positioning of the expanded device (24) relative to the blood vessel (22) structure.

Referring to FIG. 1E, with the expanded device (24) in a desirable position, the introducer catheter (2) has been removed, allowing the arteriotomy (28) to close to a greater degree as it still surrounds the attachment tension member (20) which continues to be coupled to the expanded device (24). The attachment tension member (20) may then be released or uncoupled from the deployed device (24) with a cutting tool (26), or controlled detachment configuration, such as a small mechanical latch or fitting, or a controlled release link configuration described, for example, in U.S. Pat. No. 5,122,136, which is incorporated by reference herein in its entirety. Uncoupling of the attachment tension member (20) from the device (24) allows for the arteriotomy (28) to become substantially or completely closed, leaving behind an access closure supported at least in part by the expanded device.

FIG. 1F depicts a cross sectional view of the configuration illustrated in FIG. 1E. Referring to FIG. 1F, the attachment tension member (20) is shown coupled to the expanded closure device (24) and leading out of the collapsing/closing arteriotomy (28). Subsequent to decoupling of the attachment tension member (20) from the device (24), complete hemostasis of the arteriotomy may be accomplished based upon one or more of several factors: 1) the device may be configured to bias the arteriotomy closed; 2) the vessel wall tissue defining the arteriotomy therethrough generally is self-biased to close (with a somewhat spring-like state of tissue mechanics in a vessel wall, the wall is generally biased to close when mechanically allowed to do so); 3) the device (24) may include a structure or materials which are specifically configured to prevent the flow of blood through the wall at the location of the arteriotomy. Many suitable construction variations may be utilized for the closure device (14, 24); for example, the device embodiment depicted in FIG. 1F, includes a frame comprised of frame elements or structural members (30) which are coupled to a thin, sheetlike connecting material (34) configured to be substantially impermeable to blood, and therefore a blocking element is pressed adjacent the arteriotomy (28) location to facilitate hemostasis across the arteriotomy (28) until it has healed shut. The outer surface of the structural member (30)/connecting material (34) assembly may be coupled to cover structure (32), and may be relatively thick (in one embodiment having a substantially uniform thickness of about 0.015 inches) as compared with the connecting material (34), which may be further selected for its ability to facilitate hemostasis of the arteriotomy (28). In one embodiment a suitable frame may be substantially cylindrical, with a diameter of between 6 and 14 mm, more preferably between about 11 and 14 mm, and a length of between about 12 and 20 mm, more preferably about 16 mm; with such a configuration, an associated substantially rectangular cover member may have a length of between about 16 and 41 mm, more preferably between about 19 and 24 mm, and a perpendicular rectangular dimension of between about 11 and 19 mm, more preferably about 15 mm. Suitable materials for connecting material (34) and cover structures (32) include polytetrafluoroethylene (“PTFE”), expanded polytetrafluoroethylene (“ePTFE”), polyethylene terepthalate (“PET”), polyester, polylactic acid (“PLA”), poly glycolic acid (“PGA”), poly-lactic-co-glycolic acid, fluorinated ethylene-propylene, silicone, polyethylene, polyurethane, copolymers of any of the above, other polymers, as well as porcine or equine submucosa. The structural members (30) may include metals, such as Nitinol, or polymers, such as resorbable polymers, as described below in reference to the construction of the collapsed (14) or expanded (24) forms of suitable closure devices.

In one embodiment, a cover structure may be biased to maintain a substantially cylindrical outer surface shape, and to assist in spreading loads from the structural members (30) substantially uniformly to surrounding tissue via such substantially cylindrical outer surface shape. The closure device embodiment depicted in FIG. 1F, when in the expanded configuration as shown, forms a substantially cylindrical outer shape defining a lumen therethrough; the substantially cylindrical outer shape interfaces with substantially all (i.e., approximately 360 degrees of the circumferential inner surface 238 of the vessel 22 which defines the vessel lumen 236) of the interior surface (238) of the blood vessel (22) in the region of the arteriotomy (28), including in this embodiment, the portions of this inner surface (238) which extend along the longitudinal axis of the vessel on either side of the arteriotomy (28) for a given distance each way. In other words, if an arteriotomy is created at a position approximately midway along a two-inch-long, generally cylindrical vessel portion of interest, in one embodiment, a portion of the expanded closure device may be configured to interface with substantially all of the two-inch-long, generally cylindrical, inner surface (238). In other embodiments, an expanded closure device may be configured to interface with less than substantially all 360 degrees of this circumferential surface. For example, Referring to FIG. 1K, an embodiment is depicted that is similar to that depicted in FIG. 1F, with the exception that an expanded closure device (240) directly interfaces with approximately 90 degrees (244) of the circumferential inner surface (238) of the vessel (22). FIG. 1L depicts an embodiment wherein an expanded closure device (242) directly interfaces with approximately 135 degrees (246) of the circumferential inner surface (238) of the vessel (22). The embodiment depicted in FIG. 1G varies from that of FIG. 1F in that the cover structure (32) of FIG. 1F is configured to cover substantially the entire approximately cylindrical outer surface of the structural member (30)/connecting material (34) assembly, while the cover structure (32) of FIG. 1G is configured to cover the approximately ⅔ of the approximately cylindrical outer surface of the structural member (30)/connecting material (34) assembly closest to the arteriotomy location.

Many closure device (14) variations are suitable, with general preferred characteristics being that the device be deployable through the arteriotomy in a collapsed state, and expandable once in the targeted vessel to an expanded state which promotes closure of the hole or arteriotomy through which it was delivered. For example, in one embodiment shown in orthogonal view in FIG. 1H, the closure device may include a simple scaffold or frame constructed of bent and straight portions of elongate structural members (30) in a coiled, meshed, zig-zag, or other pattern coupled to a cover structure (32) positioned to promote hemostasis of the arteriotomy (28). Such structural members (30) may, for example, include highly flexible metallic alloys or polymeric materials, such as bioresorbable polymers. Suitable metallic alloys include nickel titanium alloys, such as the superalloy known as nitinol; other suitable materials include stainless steel, cobalt chrome, titanium, nickel, gold, tantalum, magnesium, and alloys thereof. Suitable polymeric materials include those comprising poly lactic acid, poly glycolic acid, poly(lactic-co-glycolic acid), silicone, polyethylene, polyurethane, polyesther, and copolymers thereof.

In another embodiment, such as that shown in FIGS. 1I and 1J, a suitable closure device (14) may include merely a scaffold or frame comprising bent and/or straight portions of elongate structural member (30) material in a configuration that tends to bias an arteriotomy (28) closed when in a deployed/expanded configuration by urging adjacent vessel (22) wall tissue portions (36, 38) toward each other by means of small hooks, or high-friction or protein binding materials that are configured to attach or adhere to the inside of the vessel wall.

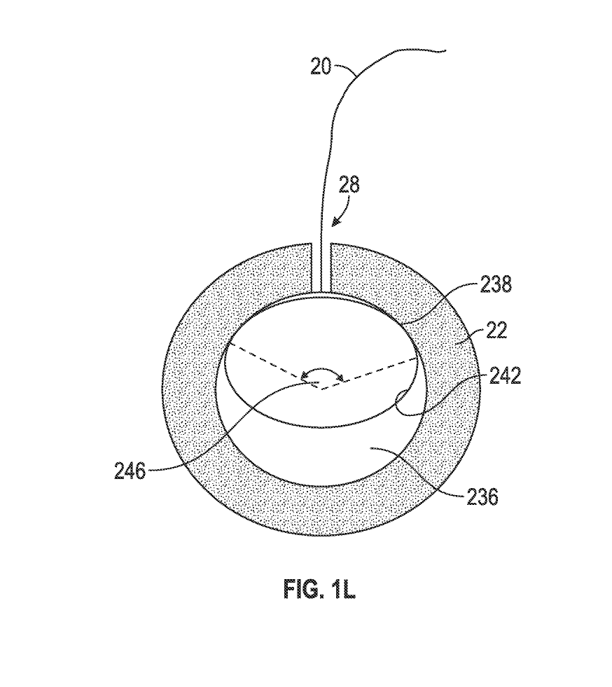

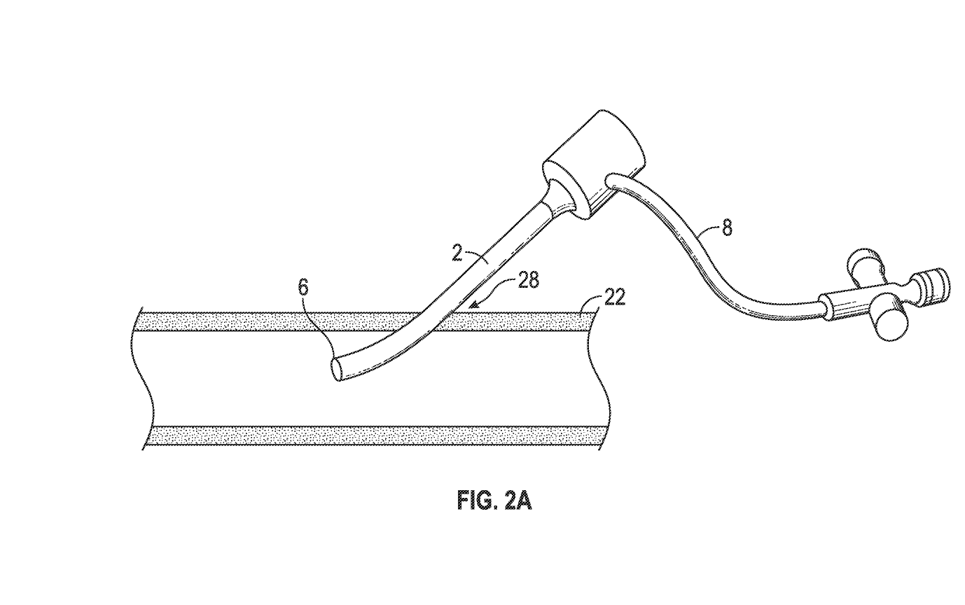

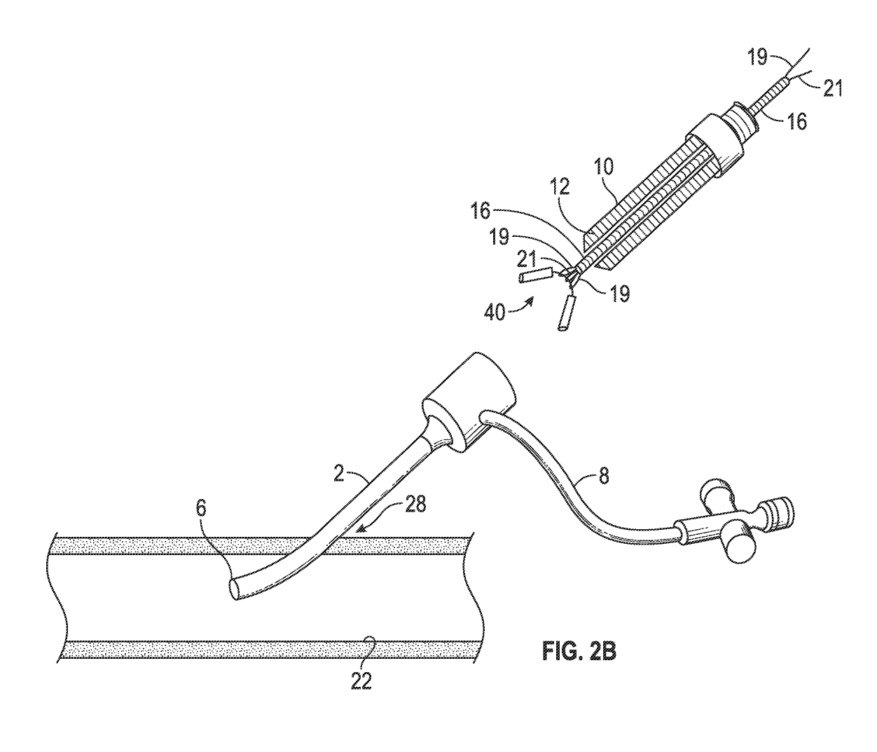

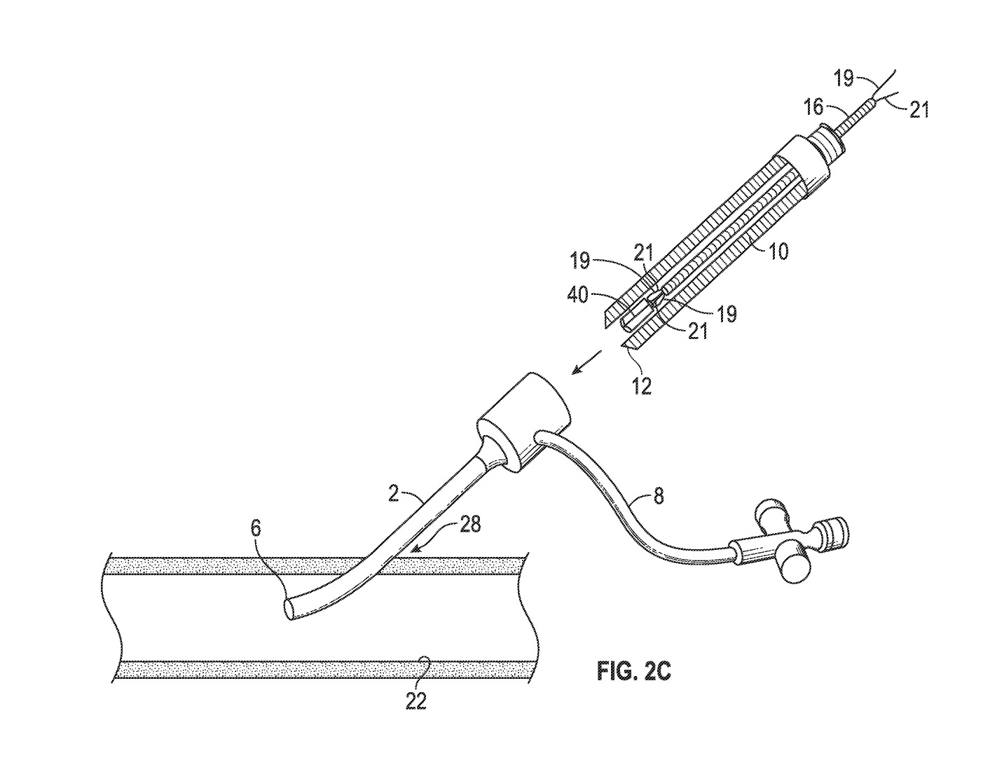

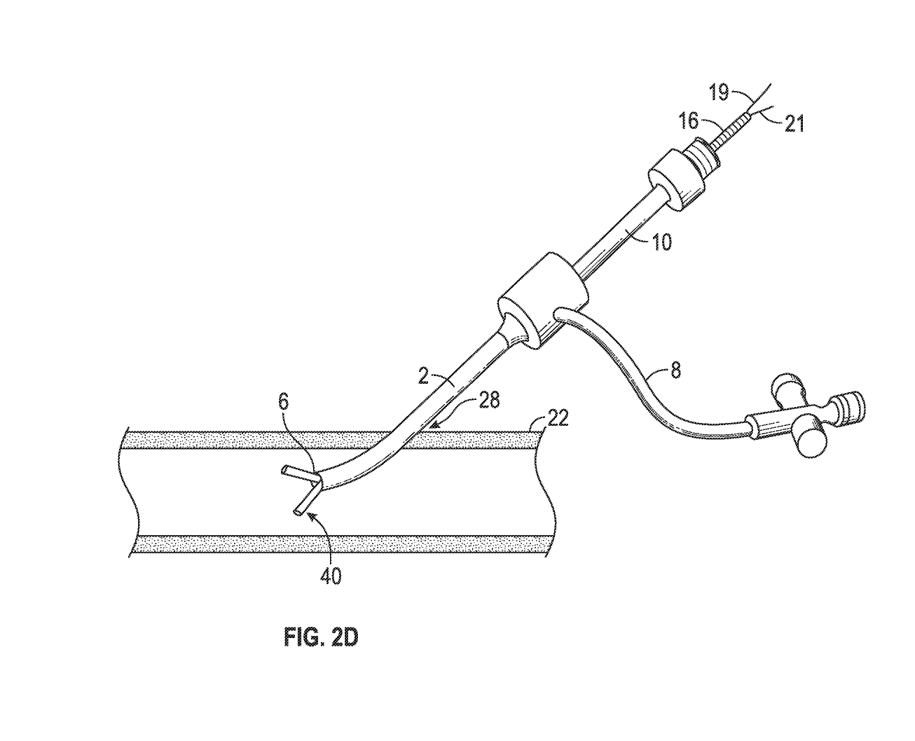

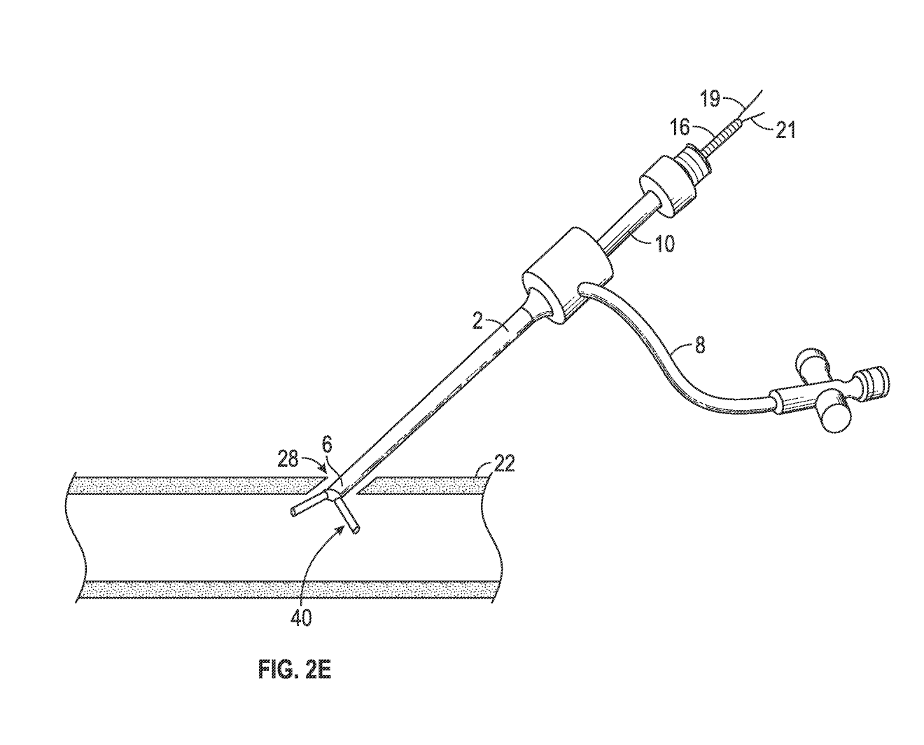

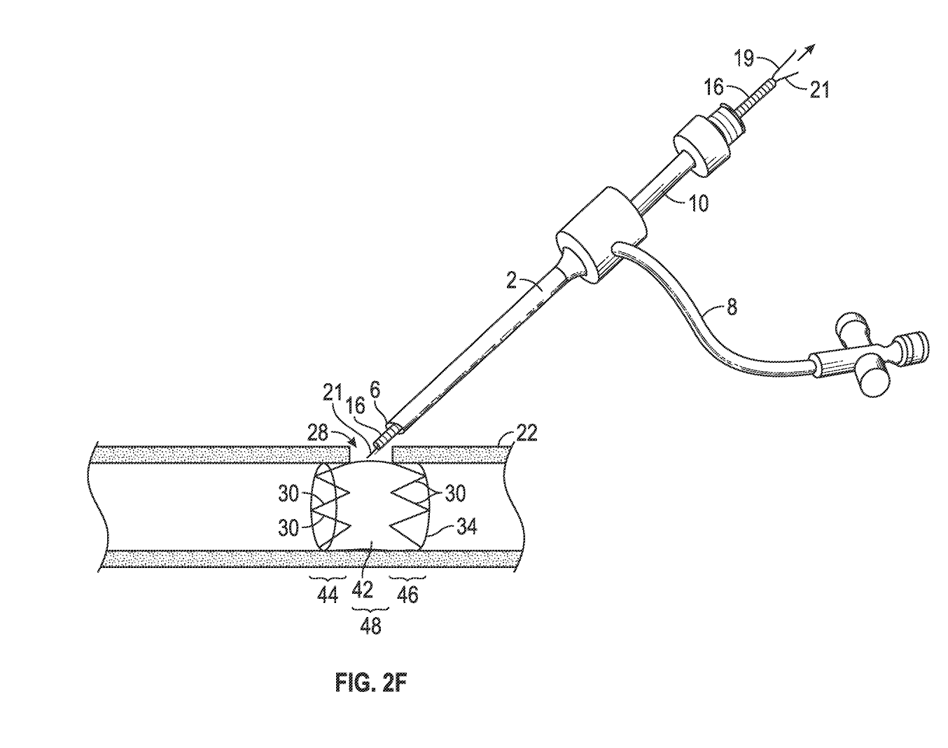

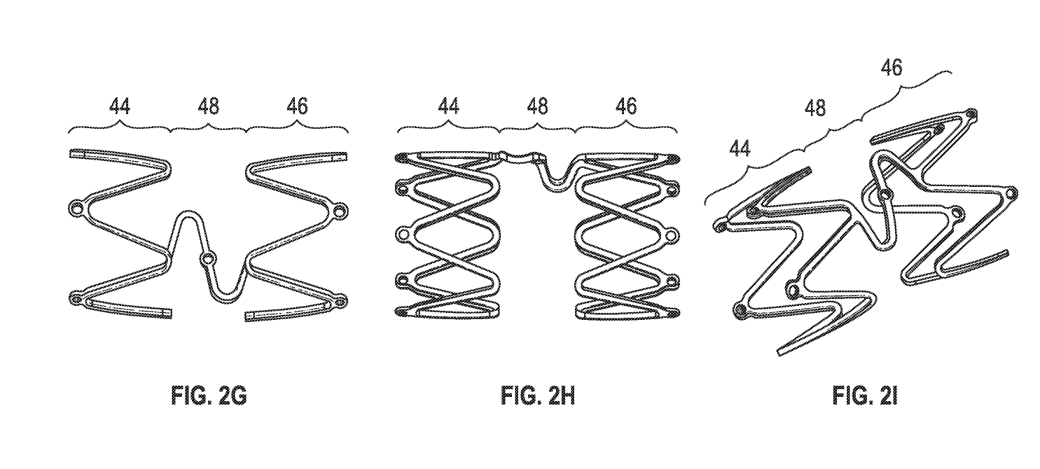

In another embodiment, a closure device (14) may include an expandable scaffold or frame such as an intraluminal stent or stentlike structure. The stent may be self-expanding or balloon-expandable and may be a stent configured for any blood vessel including coronary arteries and peripheral arteries (e.g., renal, Superficial Femoral, Carotid, and the like), a urethral stent, a biliary stent, a tracheal stent, a gastrointestinal stent, or an esophageal stent, for example. More specifically, the stent may be, for example, a stent available commercially as a Wallstent, Palmaz-Shatz, Wiktor, Strecker, Cordis, AVE Micro Stent, Igaki-Tamai, Millenium Stent (Sahajanand Medical Technologies), Steeplechaser stent (Johnson & Johnson), Cypher (Johnson & Johnson), Sonic (Johnson & Johnson), BX Velocity (Johnson & Johnson), Flexmaster (JOMED) JoStent (JOMED), S7 Driver (Medtronic), R-Stent (Orbus), Tecnic stent (Sorin Biomedica), BiodivYsio (Biocompatibles Cardiovascular), Trimaxx (Abbott), DuraFlex (Avantec Vascular), NIR stent (Boston Scientific), Express 2 stent (Boston Scientific), Liberte stent (Boston Scientific), Achieve (Cook/Guidant), S-Stent (Guidant), Vision (Guidant), Multi-Link Tetra (Guidant), Multi-Link Penta (Guidant), Multi-Link Vision (Guidant), Gianturco-Roubin FLEX-STENT®, GRII™, SUPRA-G, or V FLEX coronary stents from Cook Inc. (Bloomington, Ind.). Some exemplary stents are also disclosed in U.S. Pat. No. 5,292,331 to Boneau, U.S. Pat. No. 6,090,127 to Globerman, U.S. Pat. No. 5,133,732 to Wiktor, U.S. Pat. No. 4,739,762 to Palmaz, and U.S. Pat. No. 5,421,955 to Lau, each of which is incorporated by reference herein in its entirety. Suitable expandable stentlike structures are also disclosed in percutaneous valve configuration disclosures. For example, the configuration disclosed in U.S. Pat. No. 7,445,631 to Sadra Medical, Inc., absent the valve leaflets, may be suitably used in the subject application; U.S. publication 2009/0210052 to Forster et al discloses (for example, FIGS. 2A-2C) a tri-star collapsible frame configuration which may be utilized absent the valve leaflets in the subject application; each of these references is incorporated by reference herein in its entirety.

Any of the above devices, scaffolds, frames, stents, or stentlike structures may be combined with strips, sheets, or sheetlike portions of connecting material, such as PTFE or ePTFE, to form what may be referred to as a variant of a stent graft. A suitable stent graft is described, for example, in PCT Publication WO 1997-021403 to Prograft Medical, and is incorporated by reference herein in its entirety. Further, any of the aforementioned frames may be coupled to a cover structure (with or without connecting material as well) including a metal or polymer material positioned to assist in maintaining hemostasis of the arteriotomy, as depicted in FIGS. 1F-1H.

Referring to FIG. 2A-2I, another embodiment is depicted wherein a closure device (40) has two end portions coupled by a highly bendable midportion. Referring to FIG. 2A, a distal tip (6) of an introducer catheter (2) is positioned through an arteriotomy (28) formed in a blood vessel (22). In one embodiment, the arteriotomy may be as large as 18 French or larger in diameter. An access closure device deployment assembly is shown being advanced toward the introducer (2) in FIG. 2B, the assembly includes an expandable device (40) removably coupled to a deployment tension member (19) and an attachment tension member (21), each of which are movably coupled through a device insertion/retraction member (16) to a proximal location where they may be manipulated or controlled by an operator.

Referring to FIG. 2C, the insertion/retraction member (16) is withdrawn relative to a delivery member (10), causing the collapsed device (40) to become disposed within the distal portion (12) of the delivery member (10). Such a configuration may be inserted into the introducer catheter (2), as shown in FIG. 2D, to dispose the collapsed device (40) past the distal tip (6) of the introducer (2). Referring to FIG. 2E, the introducer (2) may be withdrawn to urge the collapsed device (40) into a position wherein it is approximately centered adjacent the arteriotomy (28) to provide a temporarily hemostasis through the arteriotomy (28). Referring to FIG. 2F, having placed the non-expanded device (40) in a desirable position as in FIG. 2E, the deployment tension member (19) may be proximally tensioned to allow the device to expand, such as by using a highwayman's hitch as described above, and the attachment tension member (21) may be subsequently uncoupled from the expanded device (42) to allow for the arteriotomy (28) to close and for the delivery instrumentation to be removed. As shown in FIG. 2F, for example, the deployed device (42) spans across the longitudinal length of the arteriotomy (28) with extra length on both sides to provide stability and leak prevention. Referring to FIGS. 2G-2I, three views of a suitable structural member frame for the deployment paradigm illustrated in FIGS. 2A-2F are depicted to show that two substantially cylindrical end portions (44, 46) are coupled by a highly flexible mid portion (48) in this embodiment, the midportion (48) preferably being positioned directly adjacent the arteriotomy location to provide support for the closure (i.e., by urging a related cover member directly against the arteriotomy location); such preferred position/orientation of the flexible mid portion (48) may be accomplished, at least in part, by interfacing a tensile member (not shown) directly with the midportion (48), for example by tying with a knot to one of the small apertures shown in the midportion (48) embodiments of FIGS. 2G-2I. Tension on such a tensile member is likely to assist with the orientation/position selection described herein, either before or after allowing the device to reach its expanded state. Such structural members may include materials similar to the structural members (30) described in reference to FIGS. 1A-1J, and may be coupled to sheetlike members and/or cover members for form a substantially cylindrical expanded device surface shape (as in the embodiment shown in FIG. 2F wherein a cover member 30 extends around each of, and between (i.e., across the midportion 48 span), the two zig-zag cylindrical frame sub-portions 44, 46, to span the arteriotomy 28 and play a key role in effecting the closure thereof), which also may be similar to those (34, 32) described above in reference to FIGS. 1A-1J. In other words, the embodiment shown in FIGS. 2A-2F, while delivered in a two-lobed collapsed form, may be expanded to form a substantially cylindrical shape due to a sheetlike member coupled across the frame, and/or a cover member extended across the outer surfaces of the frame. Individual release of an end portion (46) may be selected to support the ability to reposition the partially opened frame. Following desired positioning, the remaining end portion (44) can be expanded. Alternatively, following release of an end portion (46) the device could be safely recovered and removed from the artery. Individual release could be accomplished with use of multiple tension members (19).

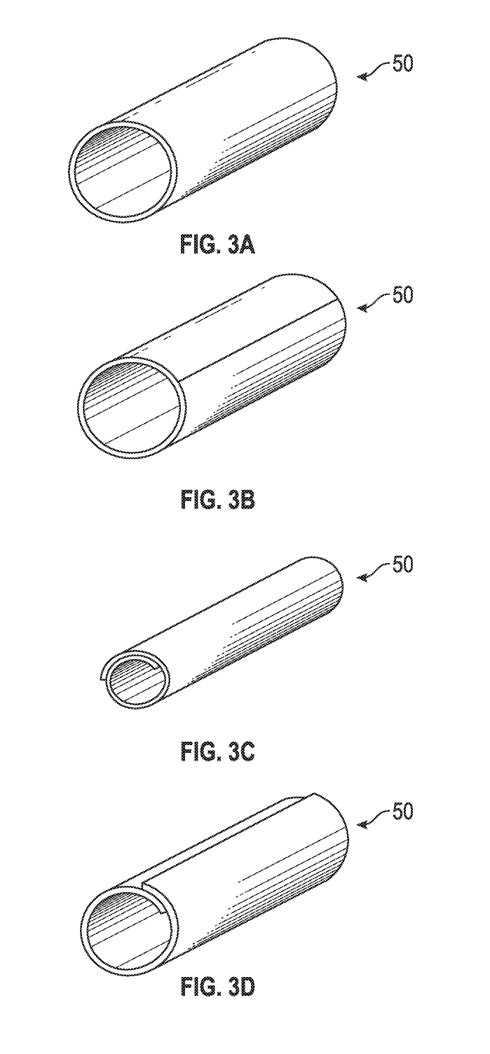

Referring to FIGS. 3A-3D, a roll-up type expandable device (5) configuration is depicted to illustrate that suitable prostheses need not be conventionally radially expandable—they may be expanded by allowing, or mechanically facilitating (i.e., with an assisting device such as a balloon or unrolling torque tool), the unrolling or unfurling of a device that has been rolled into a smaller radial configuration, as illustrated in FIG. 3C.

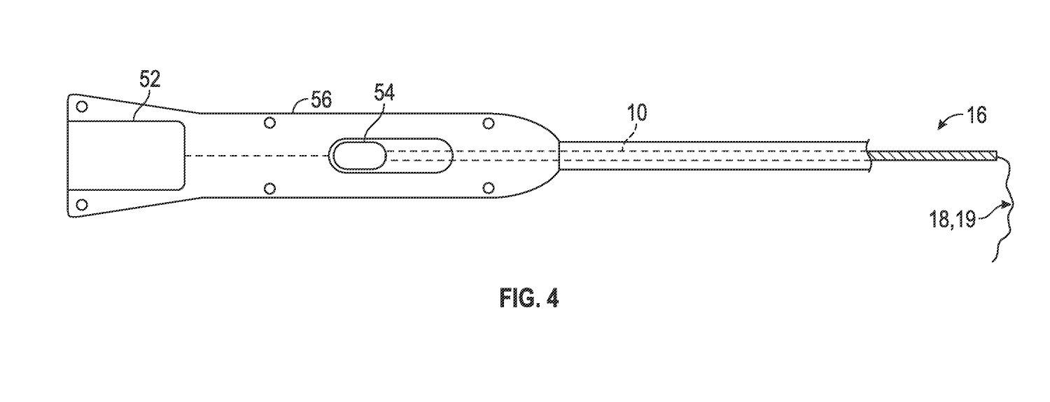

Referring to FIG. 4, one embodiment of a handle is depicted for deploying and actuating configurations such as those described in reference to FIGS. 1A-1J and 2A-I. As shown in FIG. 4, an actuator handle body (56) is movably coupled to a delivery member (16) insertion/retraction actuator slide button (54) and a deployment tension member (18, 19) tension actuation pull feature (52). Such a configuration allows for an operator to hold the handle body (56) in one hand and easily control insertion and retraction of the insertion/retraction member (16) with a thumb or finger of the same hand, while also allowing for the operator to use fingers of the other hand to pull the deployment tension member (18, 19) tension actuation pull feature (52) and allow a related device to expand or be expanded.

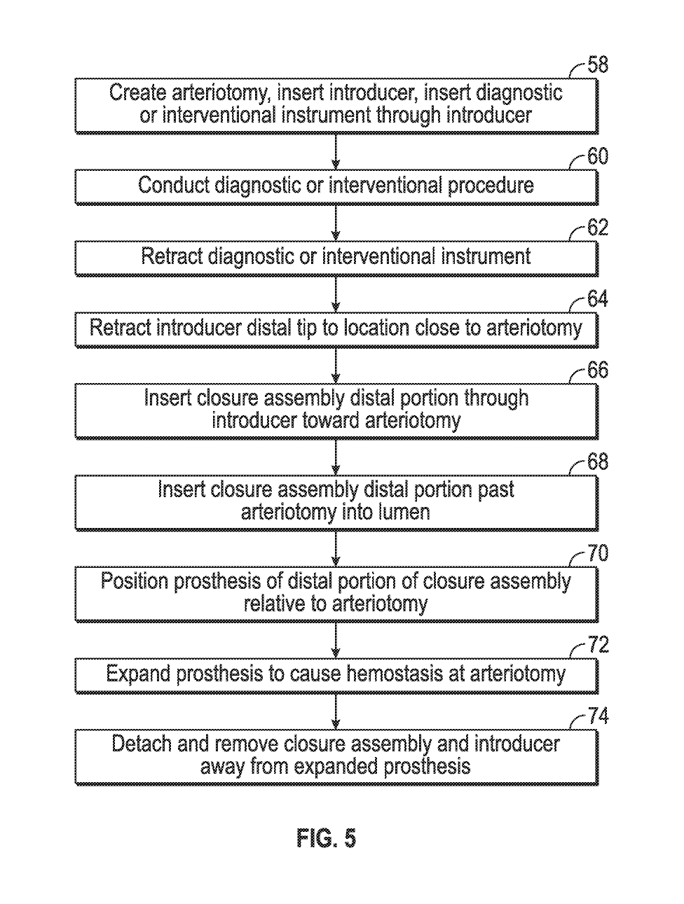

Referring to FIG. 5, a method is illustrated wherein an arteriotomy is created, an introducer inserted, and a diagnostic or interventional tool inserted through the introducer (58) to conduct a cardiovascular procedure (60) such as a percutaneous valve replacement. After the diagnostic and/or interventional tool or tools have been refracted back through the introducer (62), it may be desirable to close the arteriotomy. A distal tip or portion of the introducer may be retracted to a position close to the arteriotomy (64) but still within the vessel, and a closure assembly comprising an expandable device configured to facilitate hemostasis of the arteriotomy may be inserted through the introducer toward the arteriotomy (66). The closure assembly distal portion may be inserted past the arteriotomy and into the vascular lumen (68), after which the device may be positioned and/or repositioned to a desired location relative to the arteriotomy and surrounding anatomy (70). The device may then be allowed to expand, or may be expanded, to cause hemostasis at the arteriotomy (72), and the closure assembly and introducer may be withdrawn away from and detached from the expanded device, leaving a closed arteriotomy (74).

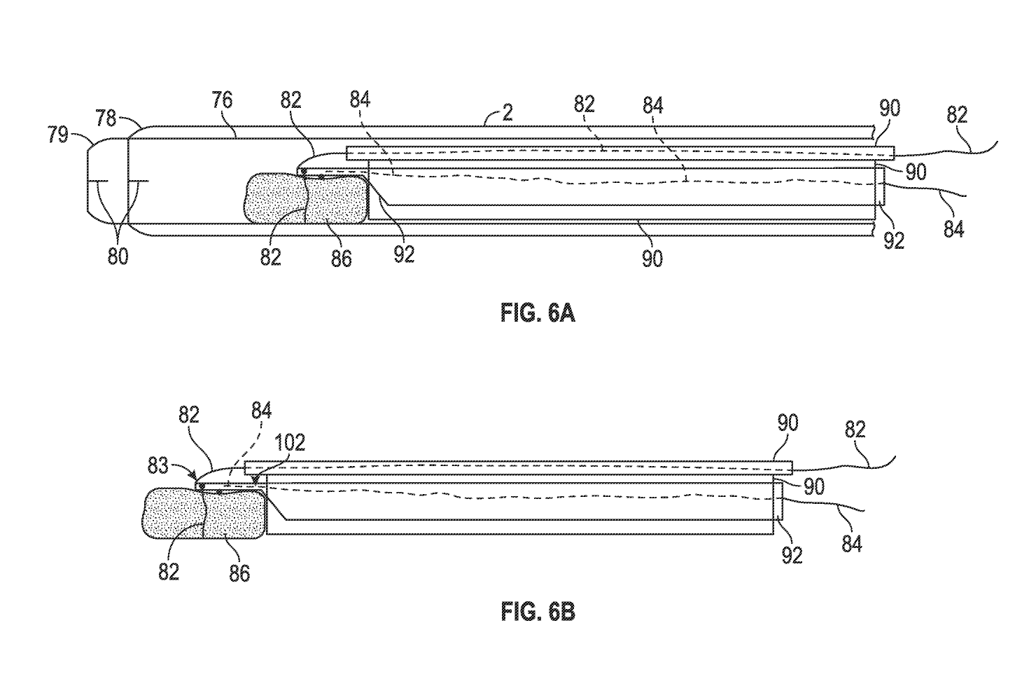

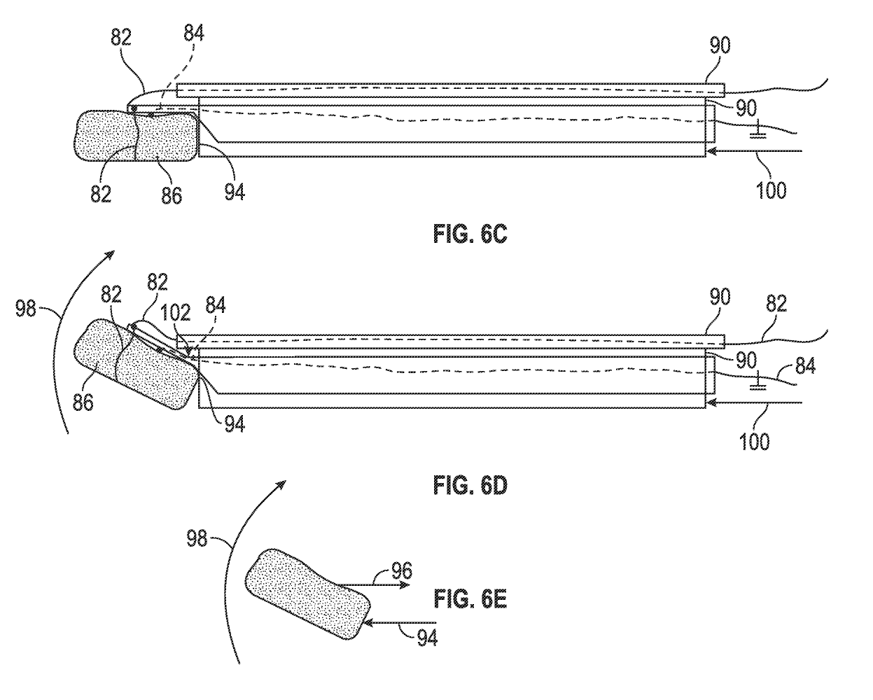

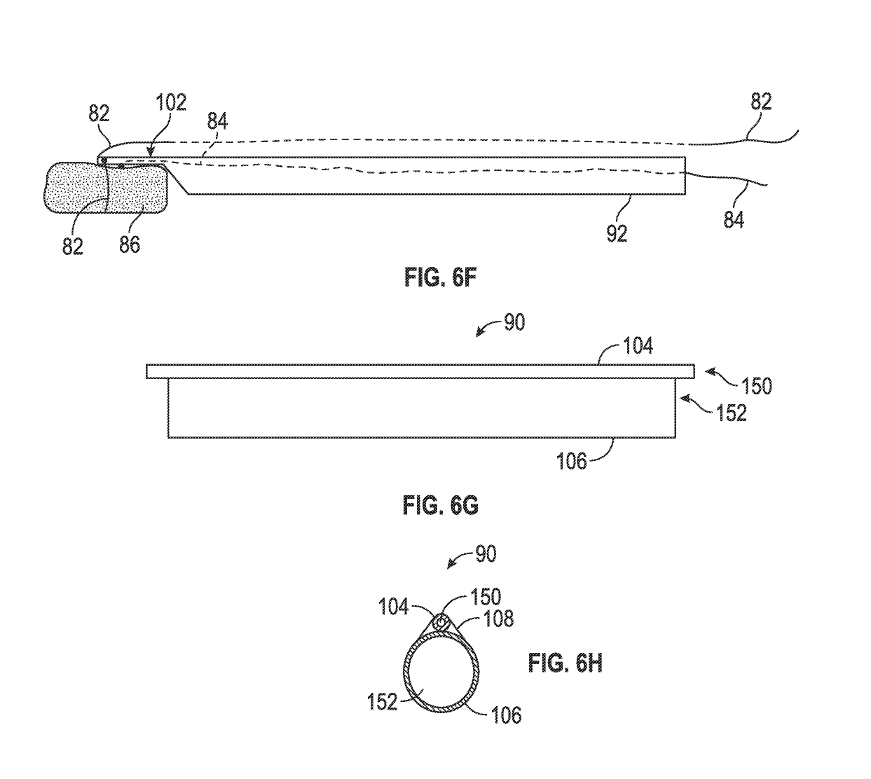

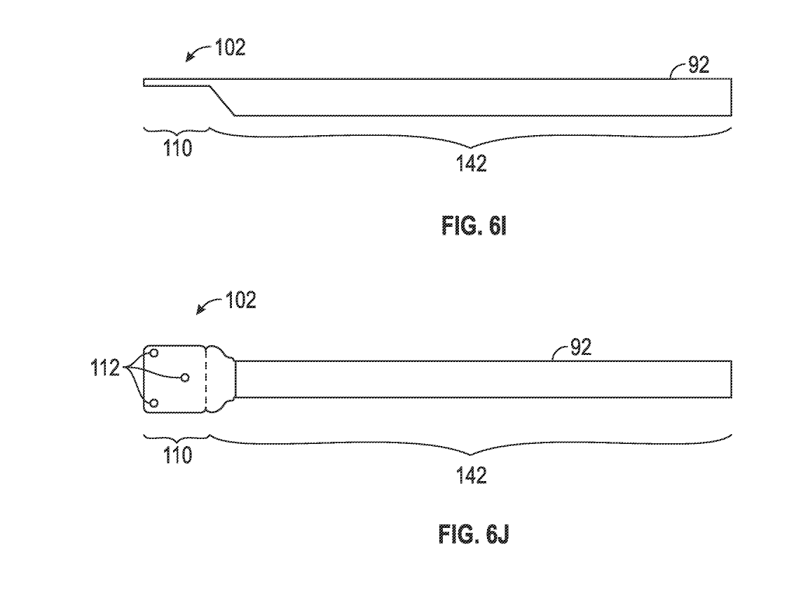

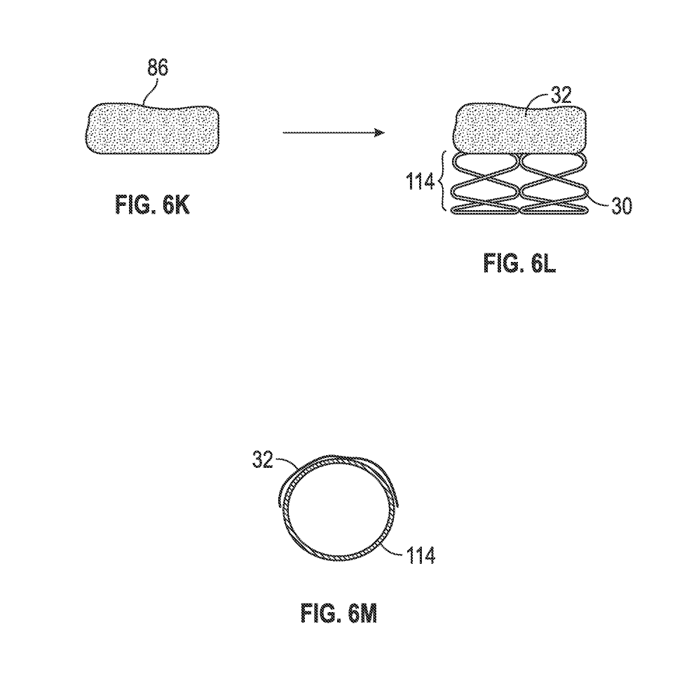

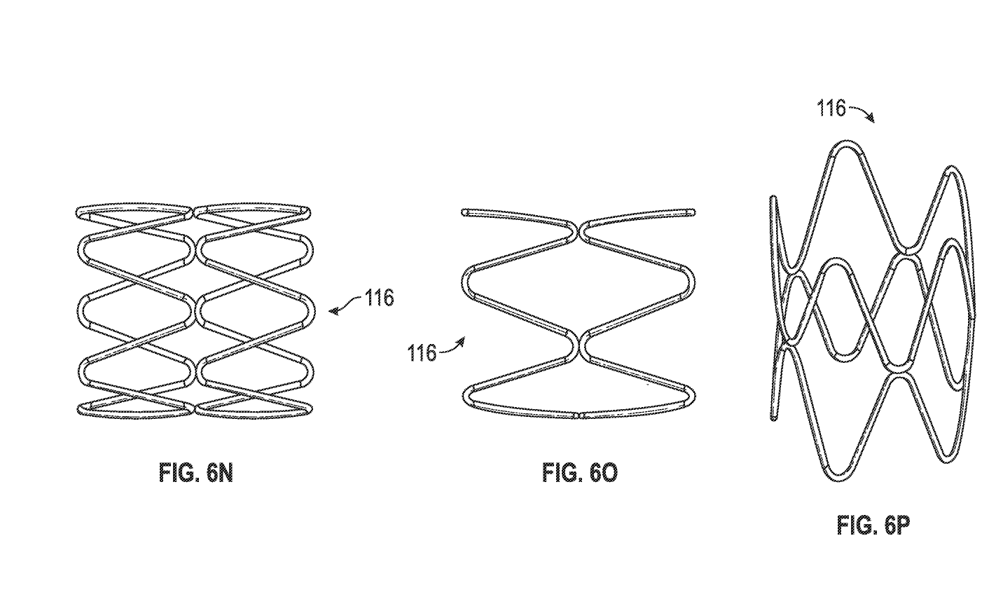

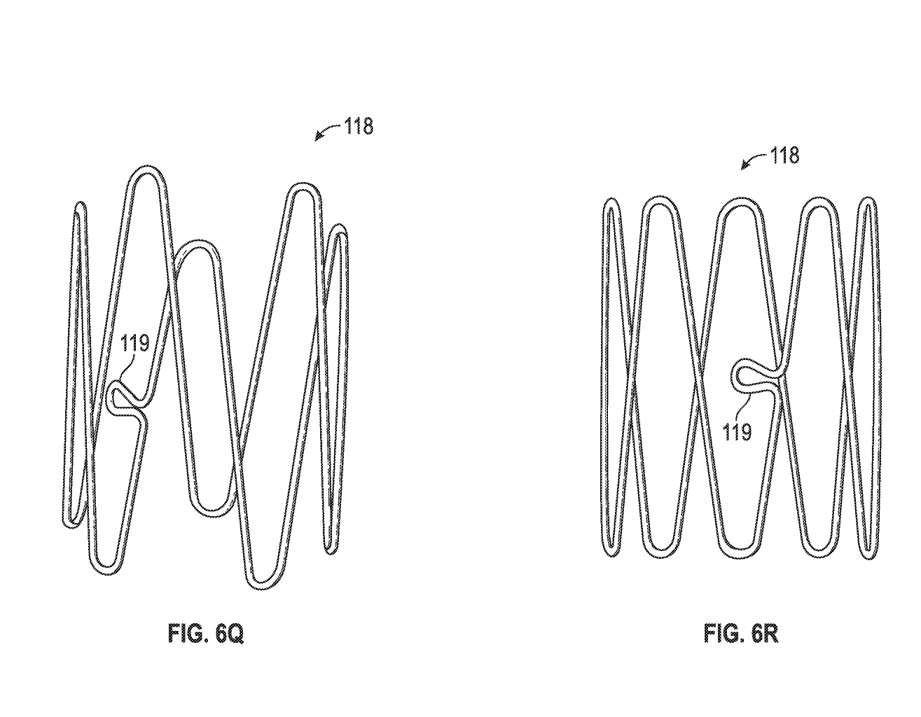

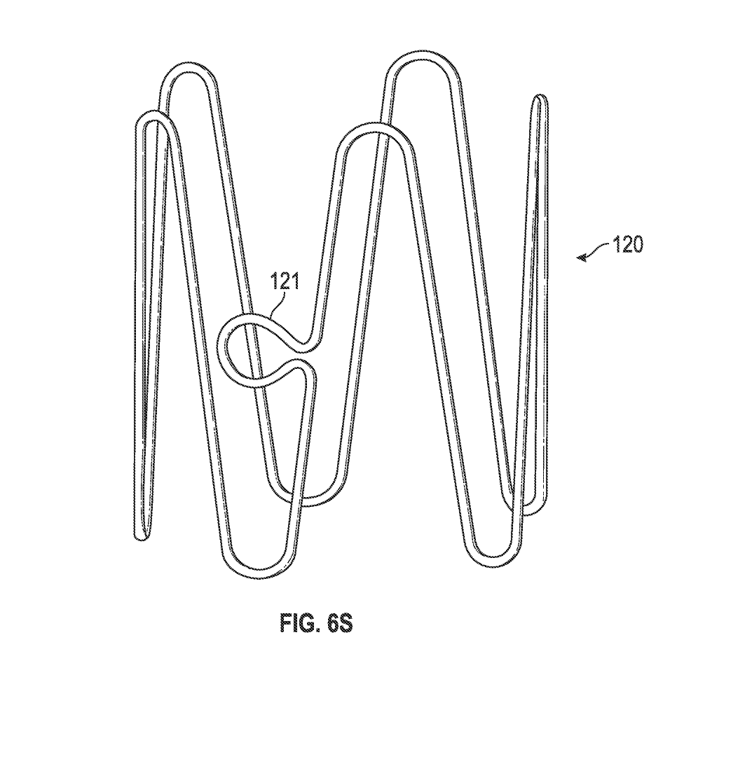

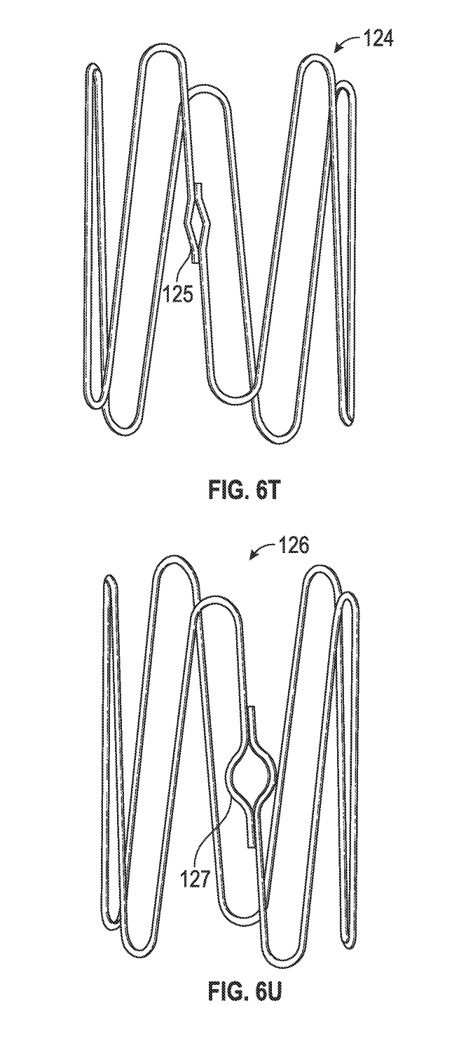

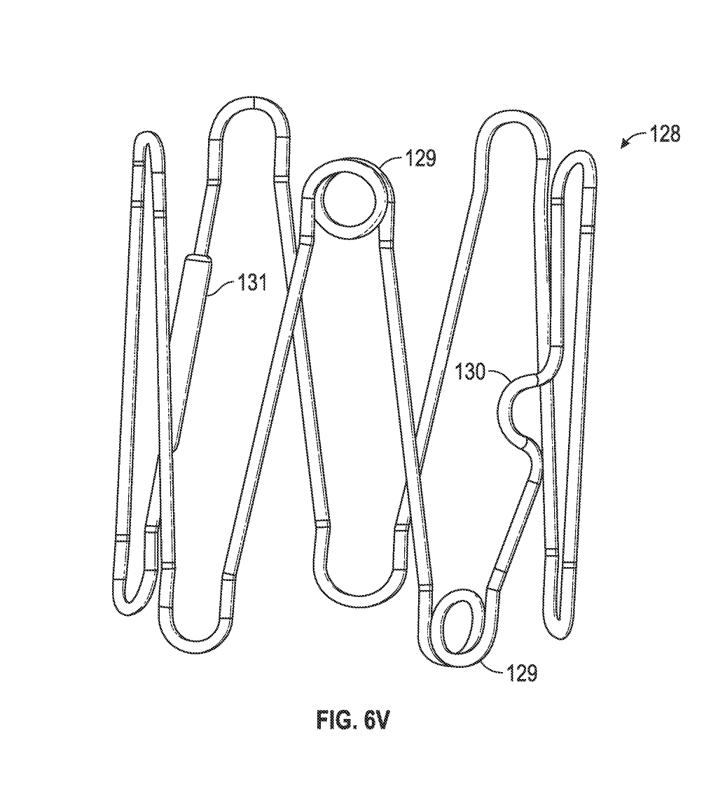

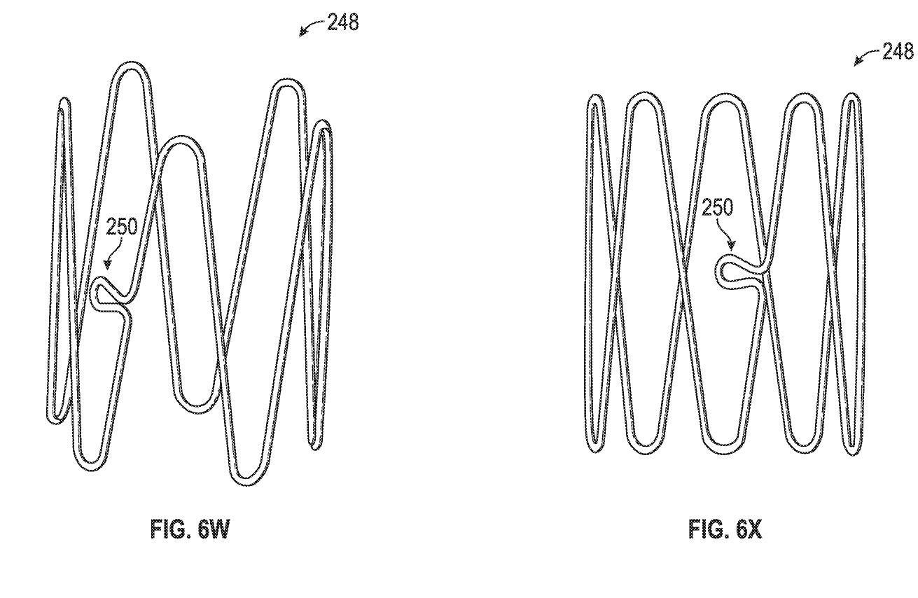

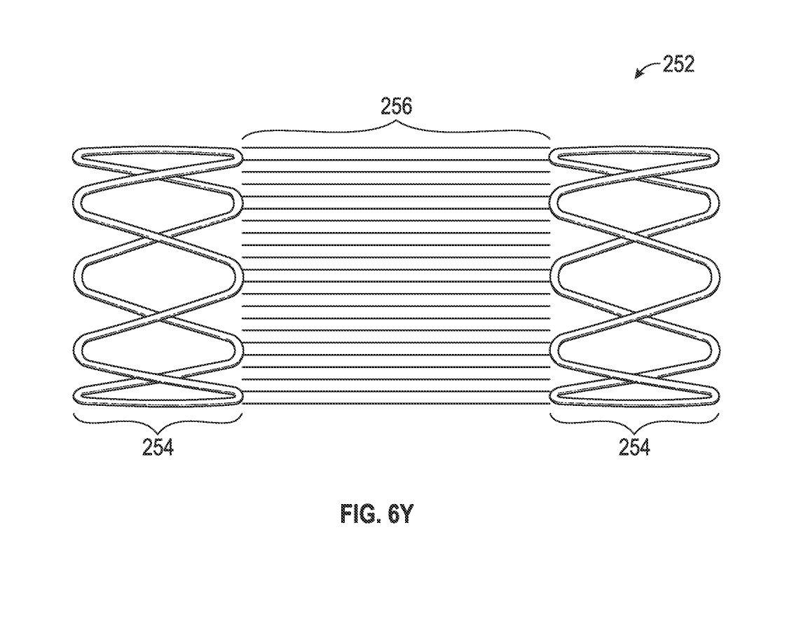

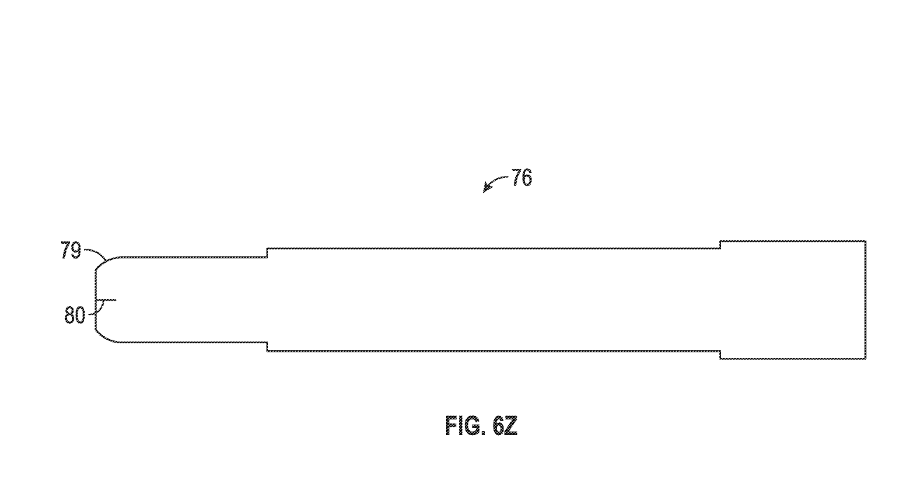

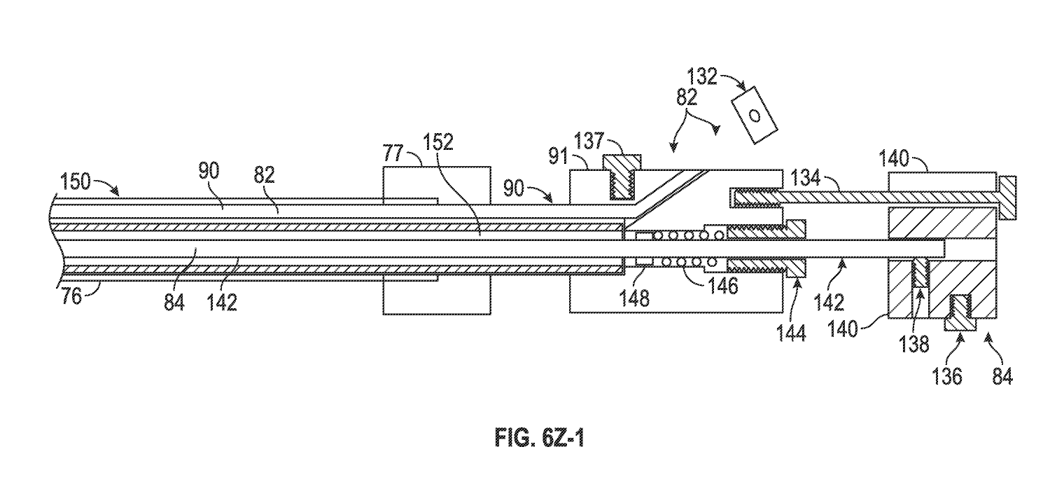

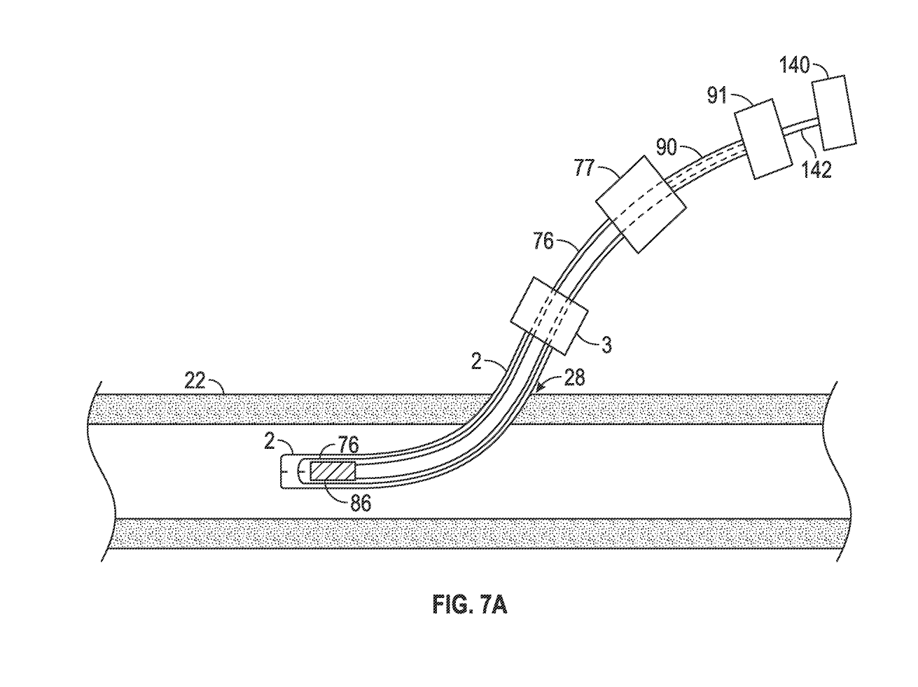

Referring to FIGS. 6A-6X, various aspects of another embodiment of a closure assembly are depicted, wherein a collapsed closure device may be controllably repositioned and/or reoriented during a deployment process in a manner that geometrically prevents such device from escaping the arteriotomy as other delivery tools subsequently are removed, and also limits or reduces blood or other fluids from escaping the arteriotomy once the device has been expanded into a final configuration, thereby effectively closing the arteriotomy. Referring to FIG. 6A, a delivery assembly is depicted including an outer introducer sheath (2), a delivery sheath (76) placed through the working lumen of the introducer sheath (2), and a arteriotomy closure device deployment assembly threaded through the working lumen of the delivery sheath (76), the assembly being depicted in FIG. 6B without the sheaths and featuring a collapsed closure device (86) coupled to both an elongate deployment member (90) and a foot member (92), the foot member being threaded through a working lumen defined by a portion of the deployment member (90). In one embodiment, the delivery sheath (76) is configured to extend beyond the end of the introducer sheath (2) by between about 200 and about 870 mm, and more preferably about 370 mm, and may have an outer diameter between about 10 Fr and about 24 Fr, and more particularly about 18 Fr. The closure device (86) may include an expandable frame, scaffold, or prosthesis with or without associated sheetlike members and/or cover members, and may be similar to those described above in reference to FIGS. 1A-3D, or below in reference to FIGS. 6K-6V. A deployment tension member (82), such as a suture or wire, is threaded through another working lumen defined by a portion of the deployment member (90) and looped around the collapsed closure device (86) as well as a portion of the foot member (92) to maintain the collapsed configuration of the closure device until the deployment tension member is tensioned, causing a releasable knot (83), such as a highwayman's hitch, to release and allow the collapsed closure device to expand or be expanded. An attachment tension member (84), such as a suture or wire that may be resorbable, akin to the attachment tension members (20, 21) described above, is threaded through a lumen or channel defined by the foot member (92) and tied to the closure device (84).

Referring to FIGS. 6C-6E, one configuration for controllably repositioning and/or reorienting a collapsed closure device (86) during deployment is configured. As shown in FIG. 6C, application of a load (100) to the elongate deployment member (90) initially will result in a compressive load at the interface (94) between the elongate deployment member (90) and the collapsed closure device (86). This compressive loading may result in translational repositioning of the closure device (86) initially until there is no more slack in the attachment tension member (84), after which a moment will be effectively applied to the closure device (86), causing it to rotate (98), or rotationally reorient, relative to the elongate deployment member, as depicted in FIGS. 6D and 6E. FIG. 6E, in particular, diagrammatically illustrates that a compressive interfacial load (94) applied along with a tensile load (96) through the attachment tension member (84) attached at a different location from the interfacial load application results in a rotation actuation. In one embodiment, rotational actuation may be accomplished by both actively tensioning the attachment tension member (84) and actively pushing the elongate delivery member (90). In other embodiments, only one of such members (84, 90) may be actively loaded, with the other kept relatively stationary. For example, referring to FIG. 6D, the attachment tension member (84) is shown grounded or anchored at a proximal location, so that rotation of the collapsed closure device (86) may be induced merely with compression or pushing upon the elongate deployment member (90) after slack in the attachment tension member (84) has been eliminated. Such rotation causes bending or hinging of a distal portion of the foot member at a predetermined hinge or bending axis (102), and the amount of rotational reorientation may be physically limited by the positions of distal portions of the elongate delivery member (90).

Referring to FIGS. 6F-6H, components of the above described delivery assembly are shown disassembled to some degree. FIG. 6F depicts a foot member (92) coupled to a collapsed closure device (86) with a deployment tension member (82) and attachment tension member (84). FIG. 6G illustrates an elongate deployment member including a first guide tube (104) coupled to a second guide tube (106) with a deployment member outer layer (108), as shown in the orthogonal view of FIG. 6H. Both guide tubes (104, 106) define working lumens therethrough (150, 152, respectively). In the assembly of FIG. 6C, for example, a deployment tension member (82) may be passed through the first guide tube lumen (150), and a foot member (92), which itself defines a lumen through which an attachment tension member (84) may be passed, may be placed through the second guide tube lumen (152).

Referring to FIGS. 6I and 6J, orthogonal side views of a foot member (92) embodiment are depicted. In the depicted embodiment, the proximal portion (142) of the foot member may include a flexible tube comprising a polymer such as fluorinated ethylene-propylene or nylon, and the distal portion (110) may include a mechanically flattened continuation of such tubing configured with holes (112) to accommodate knots and fastening of a deployment tension member (element 82 in FIG. 6C, for example) and/or attachment tension member (element 84 in FIG. 6C, for example). A crease is provided to create a preferred bending or hinging axis (102) between the proximal (142) and distal (110) portions of the foot member. In another embodiment, the proximal portion (142) of the foot member may include a reinforcing material or member, such as a piece of metal hypotube, to increase the structural modulus of such portion and facilitate precise positioning and loading of such portion to maneuver the delivery assembly or portions thereof.

Referring to FIGS. 6K-6M, as described above, the closure device may be an expandable or self expanding device that is configured to be transformable from a collapsed state to an expanded state when unrestrained, in the case of a self expanding configuration, or unrestrained and actively expanded (for example, with an expansion balloon), in the case of an actively expandable configuration. Expansion of one embodiment is depicted in the transformation between FIG. 6K and FIG. 6L, wherein the collapsed closure device (86) is freed from the constraints of one or more restraining members, such as a lumen or lumens of one or more sheaths, or one or more deployment attachment members which may be looped around the collapsed configuration. FIG. 6M illustrates an orthogonal view of the expanded configuration of FIG. 6L. The expanded configuration of the depicted embodiment, illustrated in FIGS. 6L and 6M, includes an expanded form of an expandable closure device (114) featuring a cylindrical pattern of nitinol frame elements or structural members (30), coupled to a cover structure (32), similar to those described above in reference to FIGS. 1A-1J and 2A-2I. A sheetlike member may also be coupled to the frame elements to assist with arteriotomy closure, as described above. As described above, preferably the cover (32) is sized to not only contain substantially the entire structure when in a collapsed configuration, but also to provide a layer of arteriotomy closure and leak prevention when the device has been expanded and the cover (32) has been oriented directly adjacent to the location of the arteriotomy. Also as described above, the cover (32) may include a bioresorbable material, and in other embodiments, elements of the closure device (14, 24) structure may include a polymeric material which also may be bioresorbable. The cover (32) may be coupled to the closure device (114) using a clip, wire, or suture which may be looped around one of the frame elements (30) and through the material including the cover (32). Geometric features may be created in the closure device to assist with such coupling, and may be configured to allow for coupling of the cover and closure device without a clip, wire, or suture.

Referring to FIGS. 6N-6P, three different views of a closure device frame or scaffold (116) configuration including two crowns coupled together, using a weld or adhesive junction, for example, are illustrated. A clip, wire, or suture around one of the crown junctions may be utilized to couple a cover to such device.

Referring to FIGS. 6Q and 6R, two different views of a closure device frame or scaffold embodiment (118) are shown wherein a small loop feature (119) has been formed to assist with the coupling of such scaffold and a cover. FIG. 6S depicts a similar embodiment (120) having a larger loop feature (121). FIG. 6T depicts an embodiment (124) wherein a loop feature (125) is formed with an end to end structural member junction that may include welds or adhesive junctions; FIG. 6U depicts a similar embodiment (126) with a larger loop feature (127). FIG. 6V depicts an embodiment (128) having several features of interest, including an end to end crimp tube junction (131), a small bend feature (130) which may be positioned to contain or assist with coupling an associated cover member, and two pinch coil features (129) configured to retain a portion of a cover member with a pinch friction/load fit, somewhat akin to that provided to a piece of paper with a paper clip. In other embodiments, pinch coil features (129) may be created at various locations about the closure device frame or scaffold structure, in addition to the end apex locations as shown in the variation of FIG. 6V.

FIGS. 6W and 6X depict orthogonal and side views of another embodiment of a scaffold (248) including an omega type fitting (250) which facilitates attachment of one or more elongate fastening or tethering members, such as sutures coupled to the scaffold or an associated cover member using one or more knots, in a manner wherein the knots need not touch or mechanically interfere with each other, and wherein a sheetlike cover member may be closely interfaced with the scaffold (248) without the knot or other fastening means creating a large gap between the cover and scaffold (such a gap can be disadvantageous in the endovascular environment, for example, for thrombus pooling, flow turbulence, and general flow disruption reasons). In other words, each of the two sides of the omega shape (250) can be used as a knot or fastening interface, and the gap defined by the center of the omega shape (250) maintains a distance between the two fastening interfaces which keeps them from mechanically rubbing against each other, and also provides a geometric pocket for the knots or fasteners to reside, thus allowing for a more direct interfacing of an associated cover to the scaffold (248) without gaps required to accommodate an otherwise prominent fastener or knot.

Referring to FIG. 6Y, a scaffold embodiment (252) is depicted having two end portions (254), each of which may include a non-resorbable metallic coil, for example, and a central portion (256) coupling the two end portions (254), the central portion preferably comprising a bioresorbable material such as polylactic acid or other resorbable polymer or material, as discussed otherwise herein. Such an embodiment (252) may be utilized to seal a hole or other defect as described herein, for example, using a cover member or the like, and after deployment and hemostasis at the defect, the central portion (256) is configured to bioabsorb away, leaving the end portions (254) remaining in situ where they may be encapsulated by endothelial cells, and the region of the associated vessel immediately adjacent the previous location of the resorbable central portion (256) available for secondary or revision access. In other words, the vessel portion adjacent the resorbable central portion (256) may be easily reaccessed because no implantable hardware has been left behind from the previous intervention in this portion of the vessel.

In any of the embodiments of FIGS. 6N-6Y, a suitable frame or scaffold may be substantially cylindrical, with a diameter of between 6 and 14 mm, more preferably between about 11 and 14 mm, and a length of between about 12 and 20 mm, more preferably about 16 mm.

FIG. 6Z illustrates a side view of a delivery sheath (76), which may include a generally cylindrical polymeric tube with sequentially stepped down outer diameter shaping (to provide greater flexural modulus performance and shaping distally) and an atraumatic tip (79) similar to that shown in FIG. 6A (element 78 of FIG. 6A) comprising a partially hemispheric or capsular arcuate geometry with notches (80) to allow for passage of closely fit objects (i.e., by bending forward/distally one or more of the plurality of atraumatic tip flaps formed by the notches 80 and capsular shape of the atraumatic tip 79) through a working lumen which preferably is formed and defined through the sheath (76). As described above, the introducer illustrated in FIG. 6A, for example, has similar atraumatic tip features, including a notched (80) partially hemispheric or capsular arcuate geometry (element 78 of FIG. 6A) configured to accommodate the passage of relatively closely fit objects (i.e., by bending forward/distally one or more of the plurality of atraumatic tip flaps formed by the notches 80 and capsular shape of the atraumatic tip, element 78 of FIG. 6A).

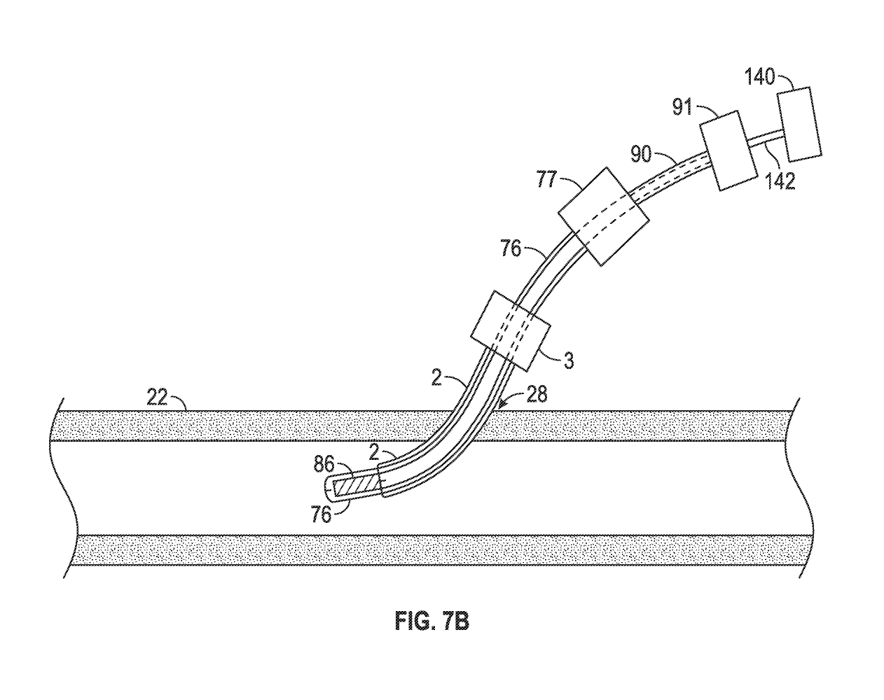

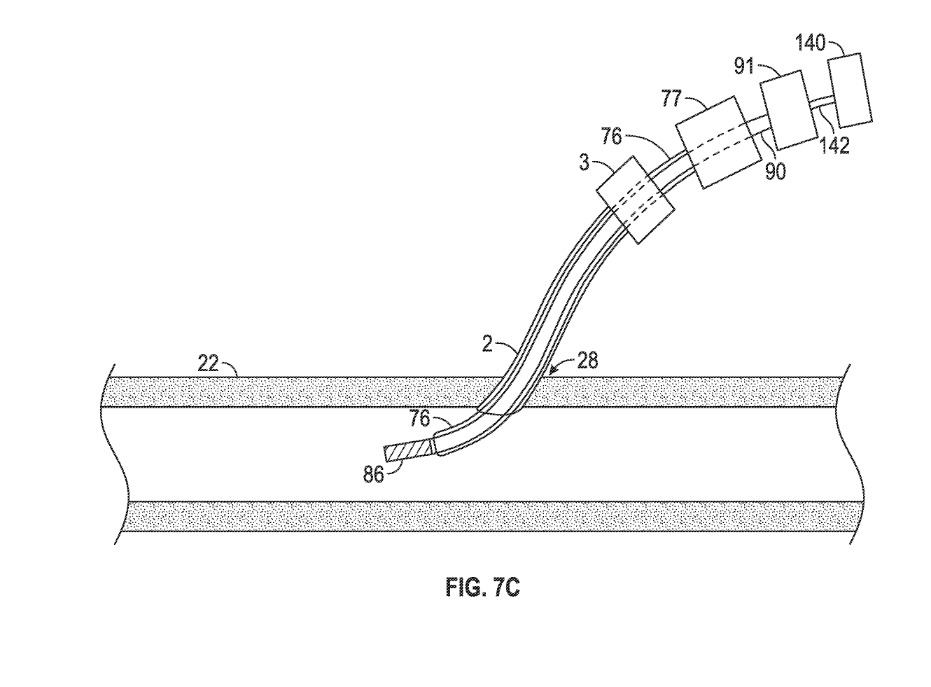

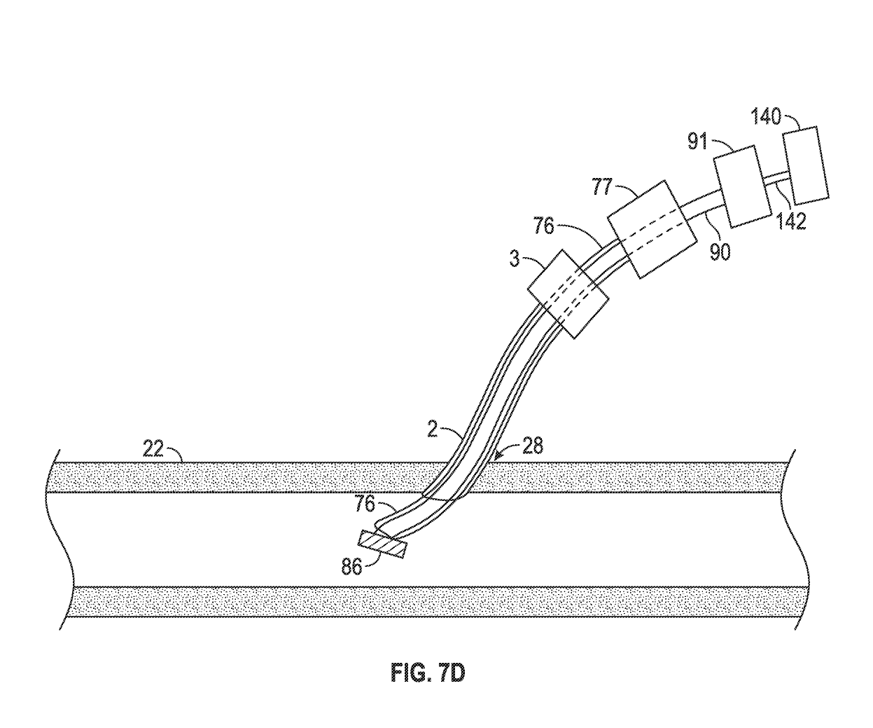

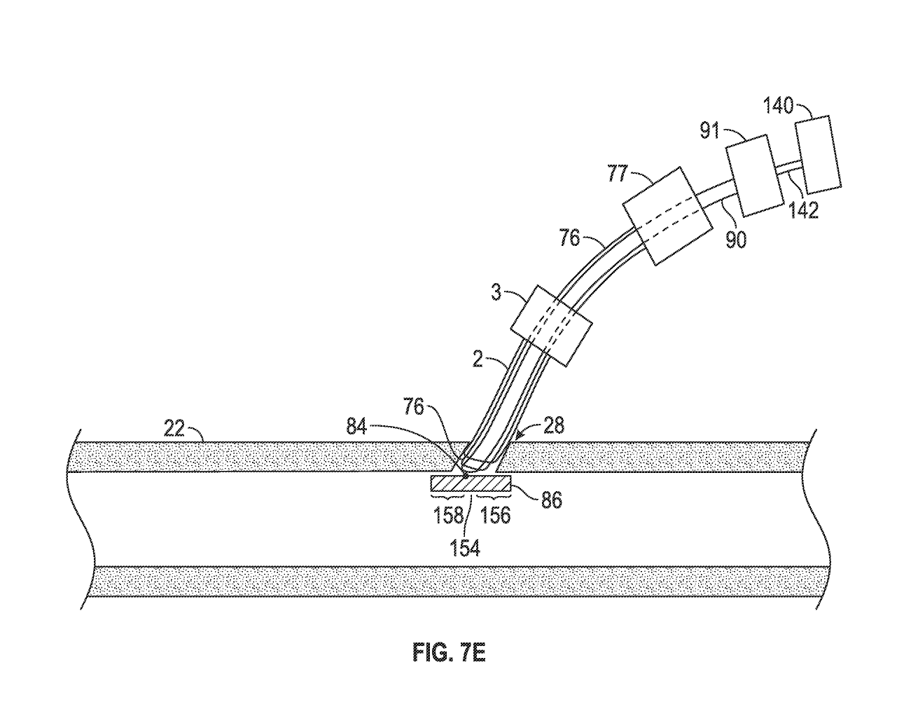

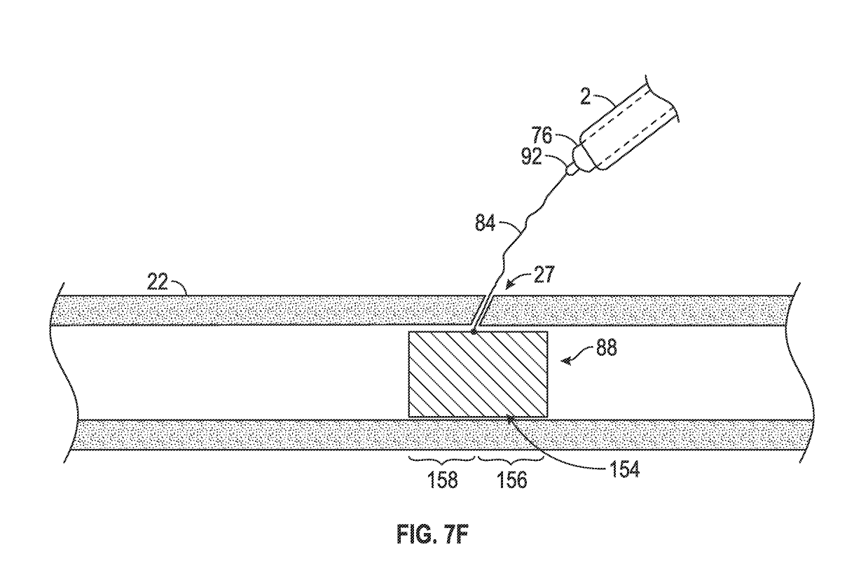

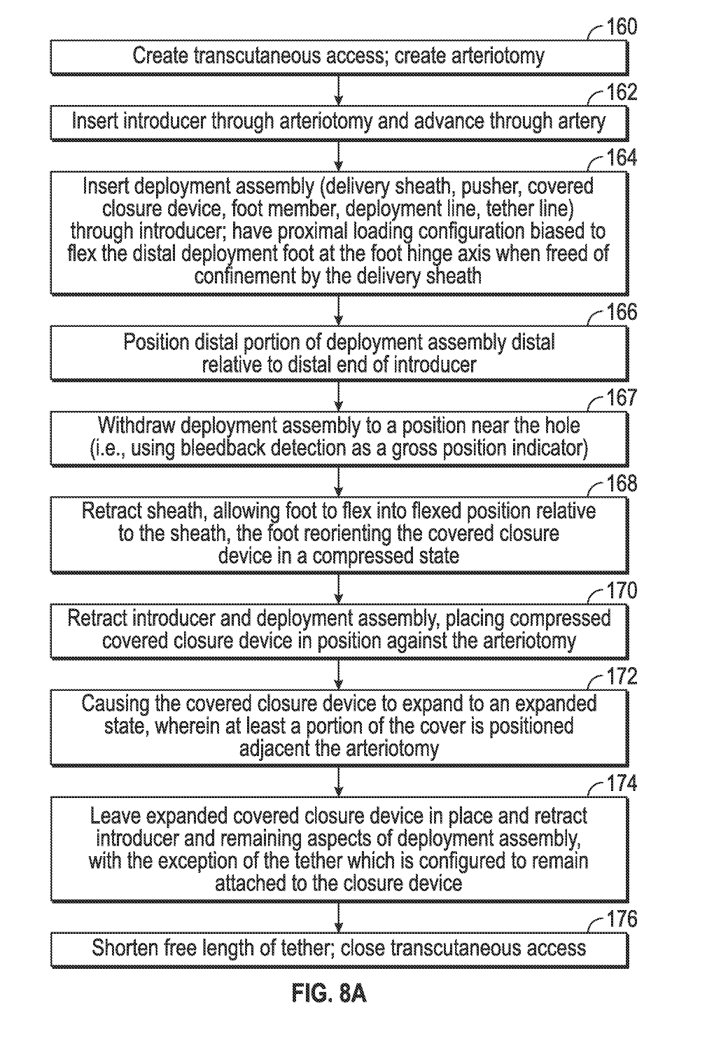

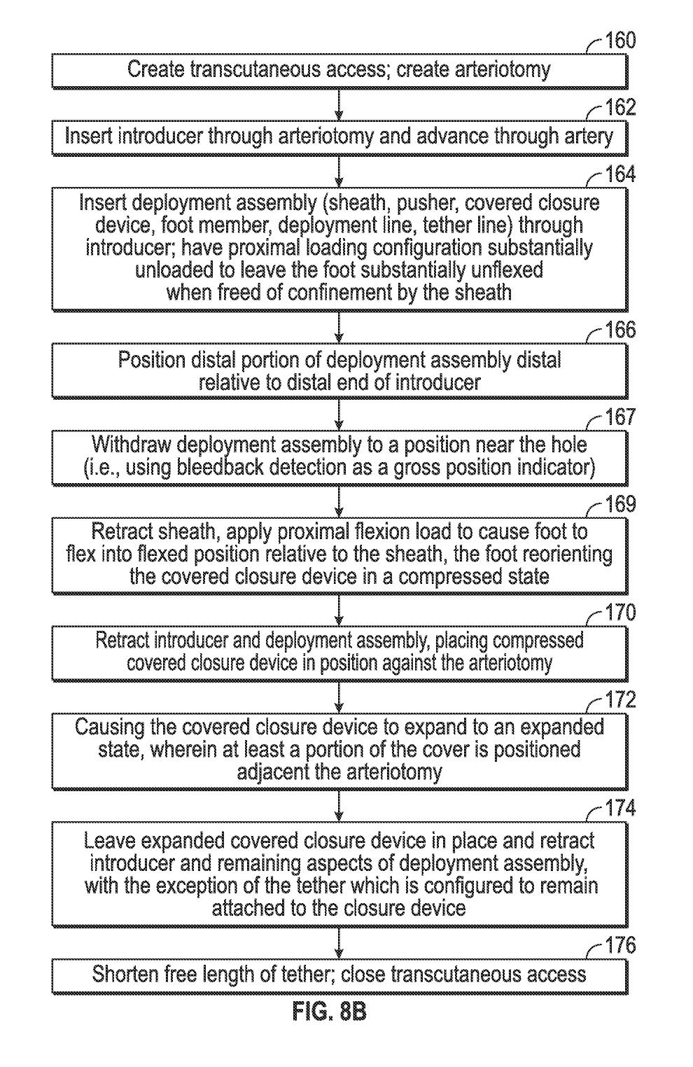

FIG. 6Z-1 illustrates aspects of the proximal portions of a delivery assembly which may be utilized with a distal deployment configuration such as that illustrated in FIG. 6A. Referring to FIG. 6Z-1, the outermost layer of the distal aspects of the depicted configuration includes the delivery sheath (76), which terminates proximally with a delivery sheath hub (77) configured to be manipulated by an operator. Within the lumen defined by the delivery sheath is a delivery assembly including an elongate deployment member (90) movably coupled to a foot member proximal portion (142). The elongate deployment member (90) terminates proximally with an elongate deployment member hub (91) configured to be manually manipulated by an operator. This hub (91) features a releasable termination screw (137) to fix one end or one portion of a deployment tension member (82), the other portion or end of which may be coupled to a pull tab (132) configured for tension manipulation by an operator to, for example, untie a highwayman's hitch knot configured to releasably contain a closure device in a collapsed configuration. The elongate deployment member hub (91) also features a screw (144) adjustable compression spring (146). The proximal portion of the foot member (142) terminates proximally in a foot hub (140) configured to be manually manipulated by an operator. A set screw (138) may be utilized to fasten the hub (140) to the foot member (142). The attachment tension member (84) is proximally routed through the proximal portion of the foot member (142) to a releasable fixation screw (136). The proximal portion of the foot member (142), which, as described above, may be reinforced by, or may include, a relatively stiff material or construct, such as a metal hypotube. In operation, when an operator wants to induce rotation and/or translation of a collapsed closure device, as described, for example, in reference to FIGS. 6C-6E, he may longitudinally reposition the elongate deployment member hub (91), foot member hub (140), and attachment tension member (84) tensioning to create such rotation and/or translation of the distal foot portion and collapsed closure device. A compression spring seat (148) coupled to the proximal portion of the foot member (142) applies loads to the proximal portion of the foot member (142) as the foot member hub (140) is pushed toward the elongate deployment member hub (91). A shoulder bolt (134) maintains the orientation of the foot member rotationally to allow the operator to understand the direction of flexion of the foot member upon desired rotation and/or translation. The compression spring adjustment screw may be tightened to pre-bias the distal foot portion to flex when released from constraining members, such as one or more sheaths which may temporarily surround and mechanically constrain the distal foot portion (i.e., thereby urging the distal foot straight as opposed to flexed). Alternatively, the spring may be left relatively unloaded to allow for release from constraining members without pre-biased flexion actuation, followed by controlled flexion actuation using the various hubs and attachment tension element. Aspects of embodiments of such operation are described in reference to FIGS. 7A-7F, and 8A-8B.

Referring to FIG. 7A, an assembly has been inserted through an arteriotomy (28) and advanced forward to place a collapsed closure device (86), such as those described above in reference to FIG. 1A-1J, 2A-2I, 3A-3D, or 6L-6V, within a blood vessel (22) and surrounded by one or more sheaths (76, 2). In some embodiments, the arteriotomy (28) may have a diameter as large as 18 French or greater. Referring to FIG. 7B, to begin deployment of the closure device (86), the introducer sheath (2) may be withdrawn proximally using an introducer manipulation hub (3), and/or the elongate deployment member (90) and foot member (142) may be advanced distally, to further expose the collapsed closure device (86). Referring to FIG. 7C, the delivery sheath (76) and collapsed closure device (86) may be moved relative to each other to further expose the collapsed closure device (86). Referring to FIG. 7D, the exposed collapsed closure device (86) may be controllably translated and/or rotated to cause rotational reorientation to a toggled position. Referring to FIG. 7E, the closure device (86) is intentionally dimensioned to not be easily passable through the arteriotomy when in the toggled position. In one embodiment, the closure device (86) may have a proximal portion, centerpoint, and distal portion along an axis parallel to a longitudinal axis of the closure device when collapsed or expanded. Preferably the total length of the device along this axis is greater than the largest dimension of the arteriotomy, and preferably the distal portion and proximal portion are long enough to prevent additional rotation of the device relative to the longitudinal axis of the vessel (i.e., about an axis perpendicular to the longitudinal axis of the vessel). In one embodiment, the attachment tension member (84) may be tensioned to place the collapsed closure device (86) against the arteriotomy with the central point (154), adjacent to which the attachment tension member preferably is terminated upon the device, aligned with the center of the arteriotomy (28), and the proximal (156) and distal (158) portions of the device extending away from the arteriotomy (28). In other words, upon controlled repositioning and/or reorientation of the collapsed closure device to the toggled or rotated position, further withdrawal of the closure device out of the arteriotomy is prevented by virtue of the geometry of the arteriotomy and closure device, the device in the depicted embodiment (86 collapsed; 88 expanded) spanning across the diameter of the previous arteriotomy location with extra length to spare on either side of this previous arteriotomy location. Referring to FIG. 7F, the deployment tension member (not shown) has been tensioned to allow the closure device to take its expanded shape (88), preferably urging the associated cover against the inside of the arteriotomy, which shrinks to a closed configuration (27) when all of the hardware has been withdrawn with the exception of the attachment tension member (84), which is configured to remain attached to the expanded device (88), and is preferably configured to subsequently biologically erode after being clipped most proximally and allowed to stay inside of the body after transcutaneous wound closure.

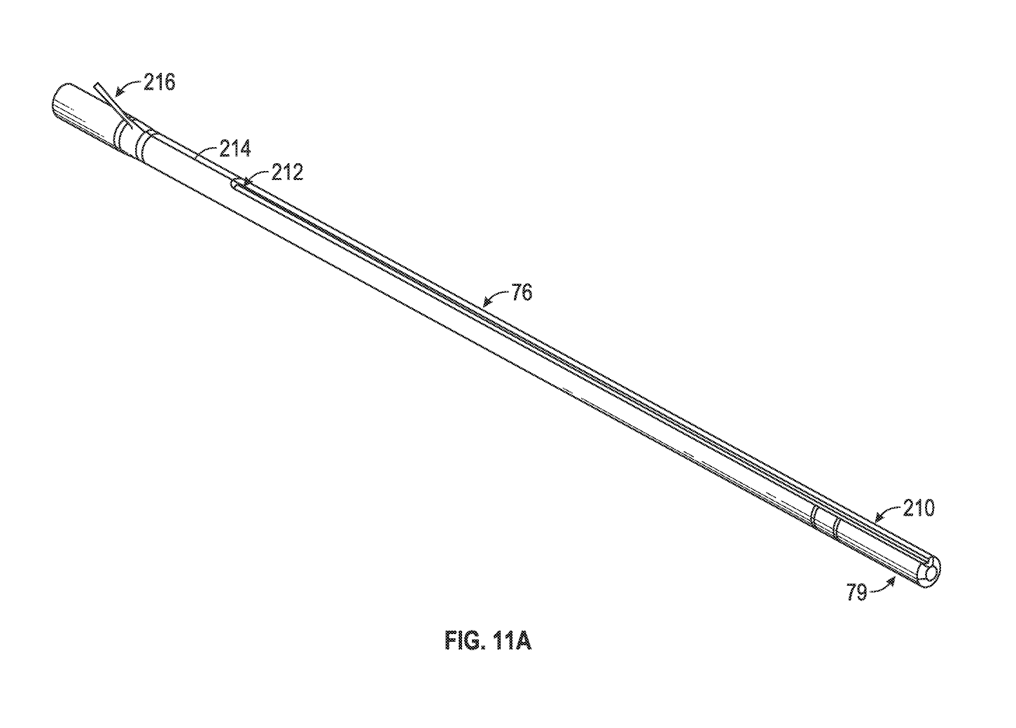

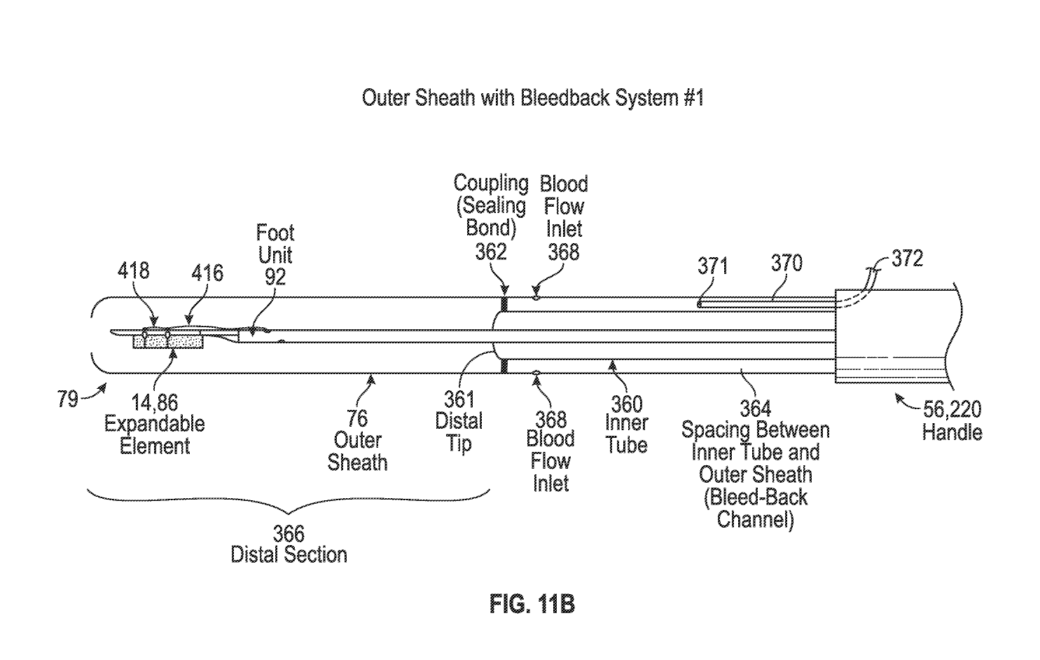

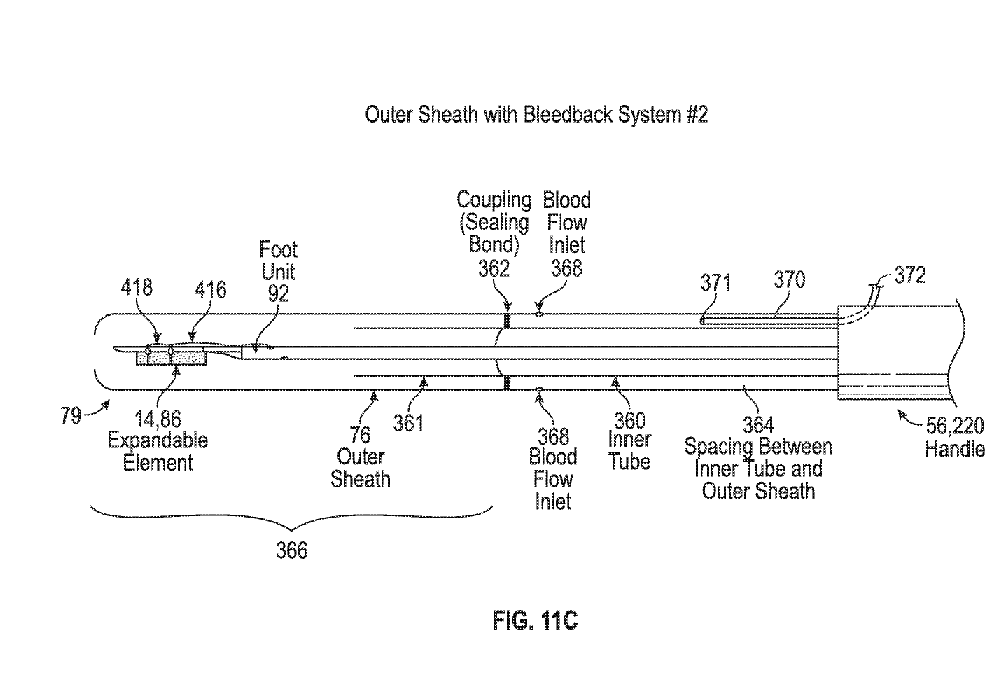

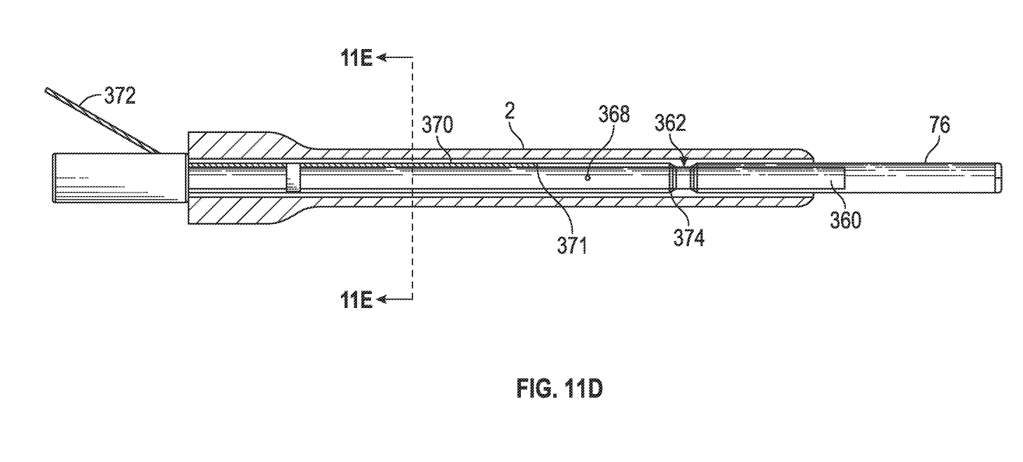

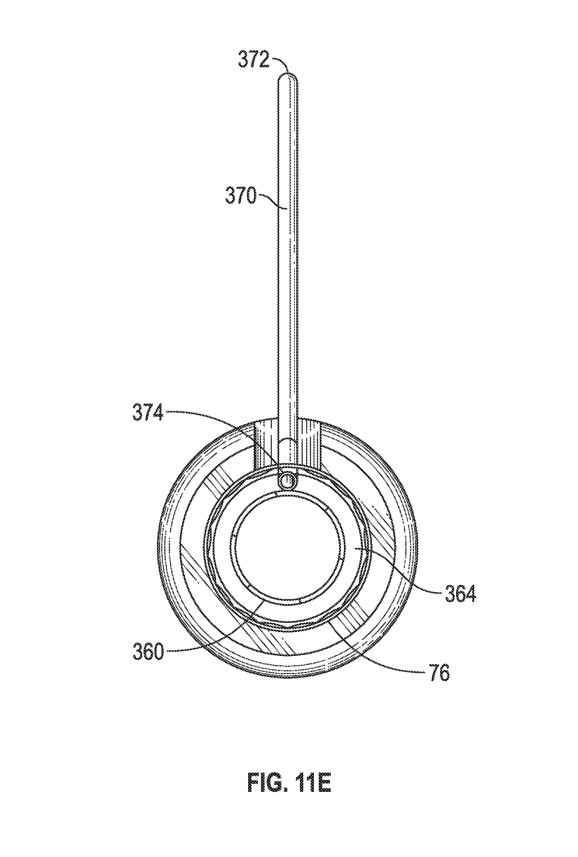

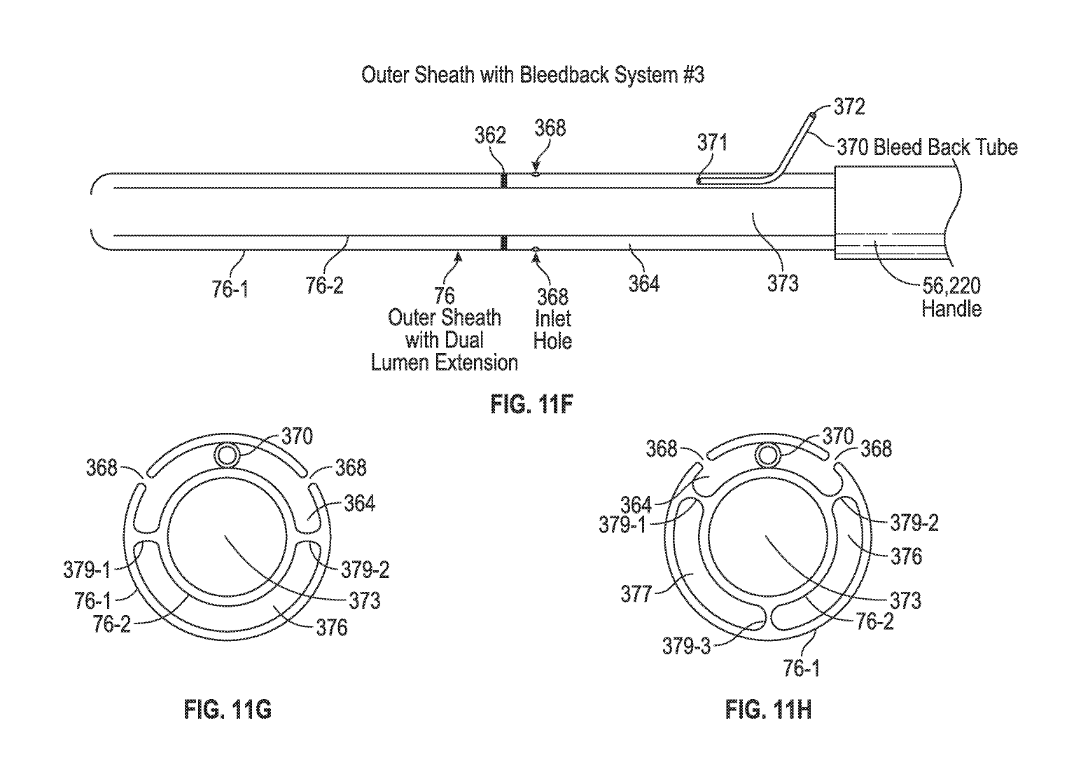

Referring to FIG. 8A, a deployment process is outlined, wherein subsequent to transcutaneous access and arteriotomy creation (160), an introducer and associated hardware may be inserted through the arteriotomy and advanced through a portion of the artery (162). Following interventional or diagnostic procedure, a deployment assembly may be advanced (164) with the introducer, or subsequently advanced, and may be exposed by moving the introducer longitudinally relative to the deployment assembly. In one embodiment wherein the foot member is pre-biased to reorient by a proximal loading configuration such as a compressed compression spring, when the distal portion of the foot member is cleared of mechanical confinement by a delivery sheath or introducer sheath, it flexes at the foot hinge or bend axis to reorient the collapsed closure device. In preparation for such reorientation, the introducer may be moved proximally relative to the closure device (166). The deployment assembly may then be withdrawn to place the collapsed covered closure device immediately adjacent the tissue structure portions around the hole in the vessel (167). This withdrawal may be facilitated by a bleed-back indicator configured to signal an operator when a predetermined amount of instrument insertion or retraction positioning has been achieved, as described below in reference to FIG. 11. In the depicted embodiment wherein the foot member is pre-biased to flex when not constrained, the last anti-flexion constraint, the delivery sheath, is withdrawn relative to the foot member, closure device, and elongate delivery member, and the closure device and distal portion of the foot become reoriented (168). Subsequently, the introducer, delivery sheath, and foot member/elongate delivery member assembly may be withdrawn to allow the closure device to be urged against the arteriotomy with tension in the attachment tensile member (170). Subsequently, the deployment tension member may be controllably tensioned to allow the closure device to expand in position against the arteriotomy, preferably with a cover portion of the closure device positioned immediately adjacent the arteriotomy location (172). The deployment and access tools may be withdrawn, leaving behind only the expanded closure device and the attachment tension member, or “tether line” (176), which may subsequently be shortened prior to transcutaneous access closure (176).

Referring to FIG. 8B, another embodiment is depicted, differing from that of FIG. 8A in that the foot member is not pre-biased to flex upon release from constraining members such as an introducer sheath or delivery sheath. In the embodiment of FIG. 8B, the collapsed closure device may be exposed to the bloodstream while the foot remains in an un-flexed configuration, and when the operator desires, may be controllably rotated and/or translated into the rotated configuration with a flexion inducing load applied deliberately (169).

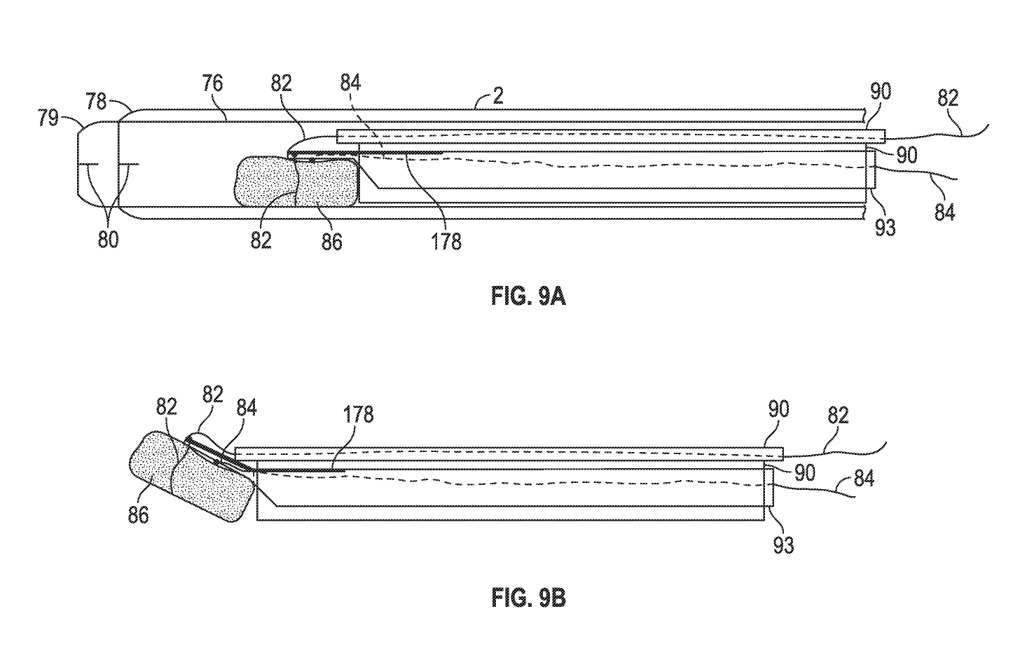

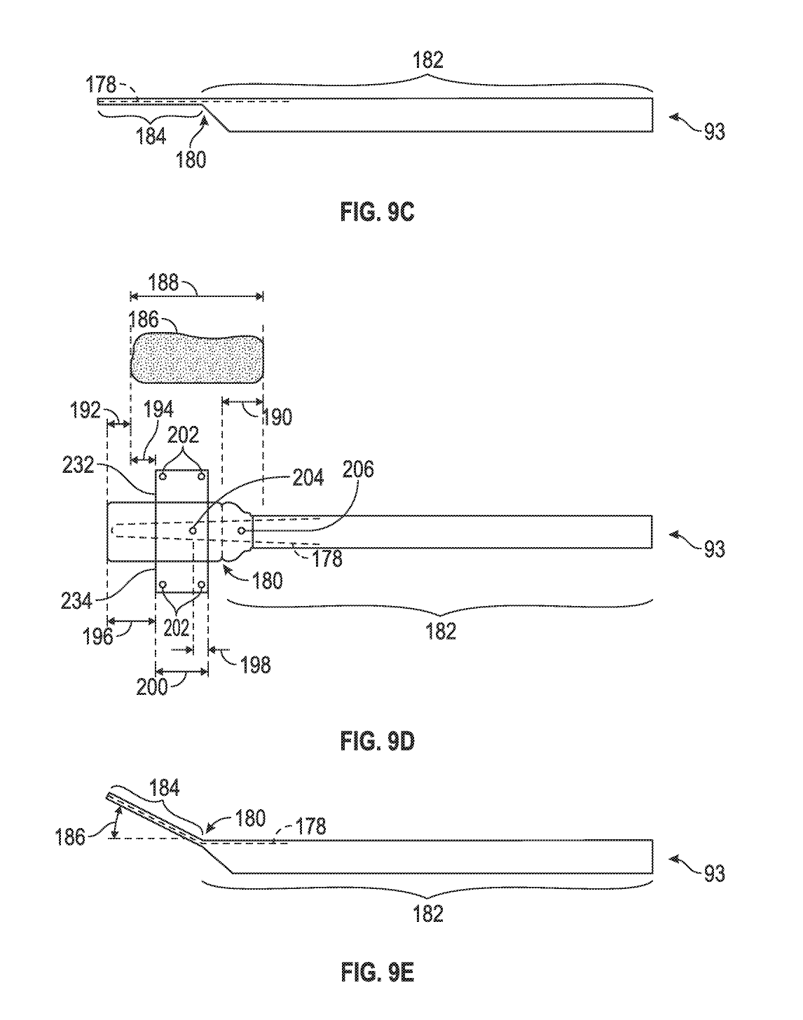

Referring to FIGS. 9A-9E, another closure device deployment embodiment is depicted, wherein an elongate bending spring member is built into the foot member to pre-bias the foot to flex when not constrained by a constraining structure such as a delivery or introducer sheath. Referring to FIG. 9A, an assembly somewhat similar to that depicted in FIG. 6A is depicted, with the exception that the foot member (93) includes an elongate bending spring member (178) extending through the distal portion of the foot member, and also through some of the distal end of the proximal portion of the foot member. Preferably the elongate bending spring member (178) includes a pre-bent metallic or polymeric member and is configured to assume a foot flexion position, as depicted in FIG. 9B, for example, when not otherwise constrained by a sheath or other constraining member to assume a straight position, as shown in FIG. 9A. In one embodiment the elongate spring member includes nitinol superalloy wire in a “V” shape as in FIG. 9D, and is coupled to proximal and distal foot member structures using interference fitting and a discrete adhesive coupling at the distal tip of the “V” shape. In other embodiments, the elongate spring member may include other biocompatible metals, such as stainless steel, cobalt chrome, titanium, nickel, gold, tantalum and/or alloys thereof, as well as biocompatible polymeric materials such as polytetrafluoroethylene, expanded polytetrafluoroethylene, polyethylene terepthalate, polyesther, polylactic acid, poly glycolic acid, poly-lactic-co-glycolic acid, fluorinated ethylene-propylene, silicone, polyethylene, polyurethane, and/or copolymers of any of the above. Further details of an elongate spring foot member are depicted in FIGS. 9C-9E. Referring to FIG. 9C, the elongate bending spring member (178) extends nearly the entire length of the distal portion (184) of the foot member (93), and a relative small length of the distal end of the proximal portion (182) of the foot member (93). When allowed to rotate (i.e., without a sheath or other constraint holding it straight), as in FIG. 9E, the foot member distal portion (184) is configured to rotate to a preselected angle (186) in accordance with the pre-shaped configuration of the elongate spring member, about an axis of rotation, or bending or hinge point, (180) that divides the proximal portion (182) from the distal portion (184). In one embodiment: the preselected angle of rotational sweep is between about 90 degrees and about 180 degrees, and more preferably about 155 degrees; the distal foot portion (184) may be between about 7 mm and about 30 mm, and more preferably about 20 mm; and the portion of the foot spring member (178) extending proximally from the flexion axis (180) may be between about 3 mm and about 15 mm, more preferably about 9 mm. Referring to FIG. 9D, an orthogonal view illustrates the placement of a collapsed closure device (86) relative to the distal portion (184) of the foot member, such distal portion (184) being in an unfolded configuration in FIG. 9D, to accommodate interfacing and coupling with a collapsed closure device (86). Four holes in the distal portion (184) of the foot are configured to assist with releasable fastening of a deployment tension member (element 82 in FIG. 9A) using a releasable knot, such as a highwayman's hitch. The distal portion (184) of the foot member may be created by crushing flat a substantially cylindrical piece of tubing comprising the proximal portion (182) of the foot member, and creating an “H”-shaped slice in such flattened portion to create the wings (232, 234) configuration depicted in FIG. 9D. Each of the wings (232, 234) is configured to be wrapped around the exterior of a collapsed closure device (86) to form a stable saddle-like interface. Two additional holes (204, 206) are formed through the foot member (93), one (204) to accommodate a deployment tension member (element 82 in FIG. 9A), and the other (206) to accommodate an attachment tension member (element 84 in FIG. 9A). Some sample dimensions for one particular embodiment include a collapsed closure device length dimension (188) of about 0.5″, a bending axis to proximal end of collapsed closure device dimension (190) of about 0.125″, a distal tip of foot member to distal end of collapsed closure device dimension (192) of about 0.25″, a distal end of collapsed closure device to distal wing edge dimension (194) of about 1/16″, a distal end of foot member to distal wing edge dimension (196) of about 5/16″, a deployment member hole to proximal wing edge dimension (198) of about 1/16″, and a wing length dimension (200) of about 0.25″.

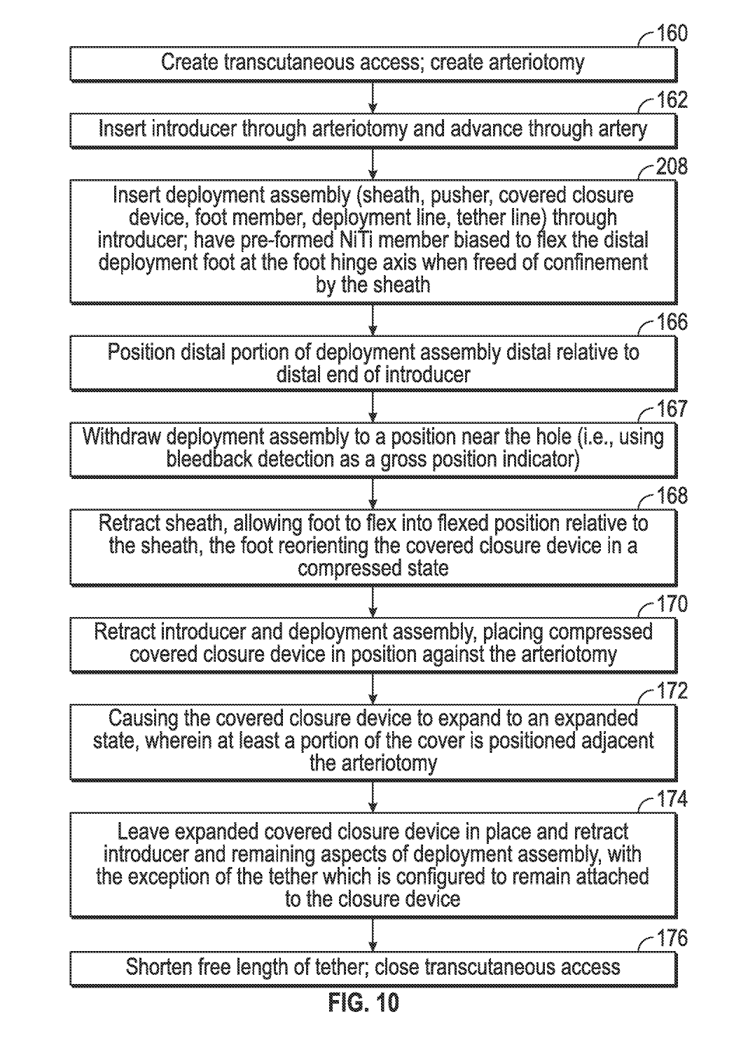

Referring to FIG. 10, a process for utilizing an arteriotomy closure system such as that described in reference to FIGS. 9A-9E is depicted, with steps similar to those described in reference to FIG. 8A, with the exception of inserting a deployment assembly wherein the associated foot member includes a pre-formed nitinol (“NiTi” or nickel titanium alloy) member biased to flex the distal foot portion at the rotational (or hinge or bending) axis when freed of confinement by a confining structure such as a delivery or introducer sheath (208).

Referring to FIG. 11A, a particular embodiment of a deployment sheath (76) is depicted, wherein a blood, or “bleed-back”, channel (210) is formed within the exterior surface of the sheath (76) to allow pressurized blood, when present at the distal tip (79) of the sheath (76), to flow from the distal end of the sheath, proximally through the channel (with an introducer sheath in place over the deployment sheath, the channel would be confined at the outer surface by the inside surface of the introducer lumen, but would remain free to flow through the channel proximally), to a lumen inlet (212), the associated lumen (214) being fluidly connected with a simple indicator fitting (216) configured to effectively ooze or squirt blood when appropriate blood pressure is present at the distal end of the channel (210). Such a configuration may be utilized in the relatively high-pressure (relative to venous) arterial system where arteriotomies are created, to provide an indication to an operator that the distal portion of the subject sheath (79) is at an insertion or refraction position wherein it is exposed to arterial flow. In one embodiment, such an indication may be utilized to position the distal portion of such sheath (79) just at the transition out of the arteriotomy and into non-pressurized space, when conducting a closure device deployment, as described above.

Referring to FIGS. 11B-H, additional example embodiments of an arteriotomy closure system having a bleed-back subsystem are shown. These example embodiments can be used as alternatives to that described with respect to FIG. 11A, and can also be used in conjunction with any embodiment of the delivery systems described herein.

In this embodiment, outer sheath (76) is fixed to an inner tube (360) via a coupling (362) such that inner tube (360) does not move relative to outer sheath (76). Coupling (362) preferably forms a fluid-tight seal to prevent passage of fluid into (and out of) the space (364) between inner tube (360) and outer sheath (76) proximal to coupling (362). Coupling (362) is preferably a thermal bond or an adhesive bond between the two adjacent tubes, an interference fit, or the like. In alternative embodiments, coupling (362) can allow inner tube (360) to slide with respect to outer sheath (76) while at the same time preventing significant fluid leakage into (and out of) space (364), e.g., using an o-ring or gasket that forms a “close fit” between the adjacent tubes.

A distal tip (361) of inner tube (360) can terminate at any location with respect to the distal tip (79) of outer sheath (76), including locations that are distal to outer sheath tip (79). In the embodiment shown in FIG. 11B, distal tip (361) of inner tube (360) terminates at coupling (362). In another embodiment, such as that depicted in FIG. 11C, distal tip (361) of inner tube (360) terminates at a position that is approximately one-third of the distance from coupling (362) to distal tip (79) of outer sheath (76). Such an extension can act to prevent kinking when components may be placed in compression or when the system is in a curved state. In both FIGS. 11B and 11C, a distal section (366) of outer sheath (76) is present distal to inner tube distal tip (361) that provides more room for housing the closure device and foot member of the arteriotomy closure system.

In the region proximal to coupling (362), the space (364) between outer sheath (76) and inner tube (360) forms a bleed-back channel. Blood is able to enter bleed-back channel (364) through one or more openings or apertures (368) (also referred to herein as blood inlets) in outer sheath (76). A bleed-back tube (370) can be bonded to outer sheath (76) such that an open distal end (or distal port) (371) of the tube (370) is located within the bleed-back channel (364). Open distal end (371) allows blood to flow from bleed-back channel (364) into bleed-back tube (370) and pass proximally through the bleed-back tube to a proximal open end (372) (proximal port or blood flow outlet) of bleed-back tube (370). Passage of blood out of this proximal open end (372) provides a visual indication to the medical professional that the blood flow inlets (368) are exposed to blood in the patient's vasculature, which in turn indicates the delivery system and introducer sheath reside within the vessel. In an alternative embodiment, bleed-back tube (370) can be omitted and a proximal bleed-back outlet port can be formed directly in bleed-back channel (364).

FIG. 11D is a side view of another example embodiment of the delivery system. Here, coupling (362) is a thermal bonded section. Introducer (2) is shown in cross-section about outer sheath (76). The delivery system and introducer are preferably configured such that bleed-back occurs when both the distal tip of introducer (2) and the distal tip of outer sheath (76) are exposed to blood flow. This can include the arrangement where blood inlets (368) are located proximal to the introducer distal tip and, although inlets (368) are not directly within the blood stream, blood nevertheless can travel proximally in the space between introducer (2) and outer sheath (76). The arrangement where inlets (368) are distal to the introducer distal tip is also possible.

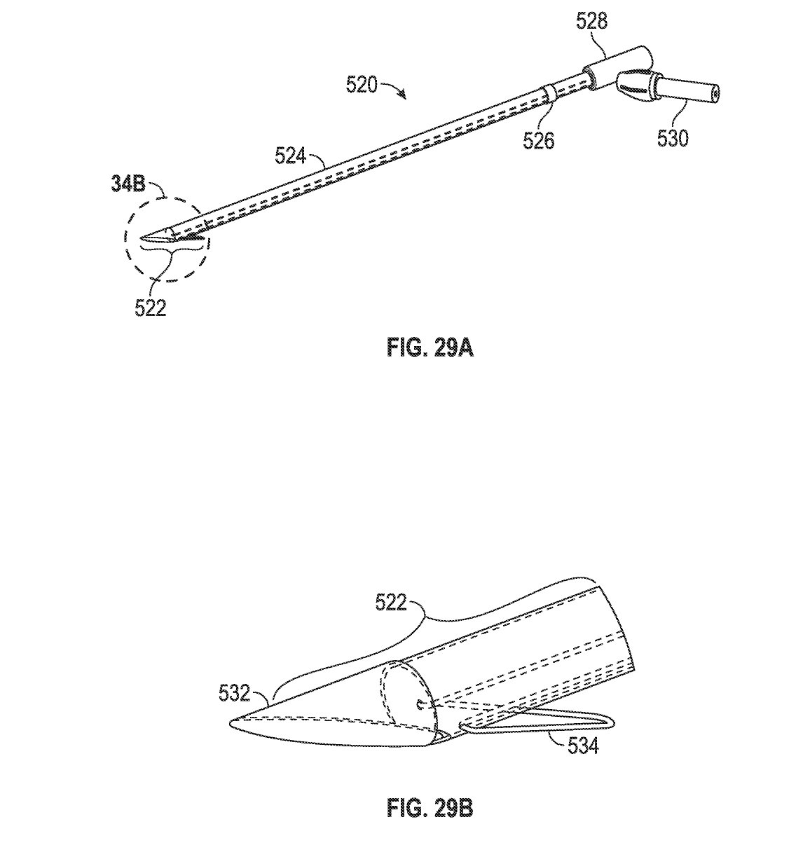

In an alternative embodiment, bleed-back may be routed through the space between the introducer (2) (also referred to as a “vascular sheath”) and the outer sheath (76). In this embodiment, blood-flow may exit the delivery system through the blood outlet or port (372), which can be coupled to a proximal hub of the introducer and operatively coupled to the aforementioned space. The blood outlet (372) can have a valve to control the rate of blood exit. This example embodiment may be used as an additional alternative to that described with respect to FIGS. 11B-11D, and may also be used in conjunction with any embodiment of the delivery systems described herein. For instance, when used with the embodiment of FIGS. 29A-B, when the introducer (2) is in place within the vasculature, the outer sheath (524) of the delivery device (520) is inserted into the introducer (2) until the marker (526) is aligned with a predetermined location on the introducer (2), such as its proximal termination. At this point, a predetermined amount of the distal portion (522) of the outer sheath (524) is exposed past the distal termination of the introducer (2). The blood outlet (372) is preferably open at this point to allow blood to exit the outlet and form a visual indication that the delivery device (520) is within the vasculature. The delivery device (520) and introducer (2) are then slowly pulled back (proximally) together, preferably without moving either with the respect to the other to maintain the appropriate spacing, until bleed-back through the blood outlet (372) stops. Cessation of bleed-back is indicative of the movement of the introducer (2) from a position within the vasculature (where blood is free to travel through the space between the delivery device (520) and the introducer (2)), to a position past the arteriotomy and outside of the vasculature (where blood is no longer exposed to the space between the delivery device (520) and the introducer (2)). Because the distal portion (522) of the outer sheath (524) is exposed past the distal termination of the introducer (2), a portion of that exposed sheath (524) will remain within the vasculature, in a position where the distal termination of the outer sheath (524) (and any catch member) is in close proximity to the arteriotomy. From this point, deployment of the closure device can begin.

FIG. 11E is a cross-sectional view of the delivery system taking along the lines 11E-11E of FIG. 11D. Although not shown, a proximal air release valve (which can also be configured as an opening or microvent) is present in outer sheath (76) at or near handle (56, 220). This valve allows air to escape as blood enters bleed-back channel (364) through blood flow inlets (368).

Any number of one or more inlets (368) can be used for blood entry into bleed-back channel (364). Bleed-back inlets (368) can be located at regular intervals about outer sheath (76) or can be spaced irregularly. In one embodiment, four inlets (368) are present and located at regular intervals (e.g., offset 90 degrees from each other). The presence of multiple inlets (368) about the circumference of sheath (76) allows blood to enter bleed-back channel (364) even in tortuous conditions, such as where sheath (76) is pressing against an inner surface of the introducer. Inlets (368) are preferably located relatively near or adjacent to coupling (362). For instance, inlets (368) could be located in a distal-facing surface, such as surface (374) of outer sheath (76) in FIG. 11D (as opposed to in the sidewall as depicted). Distal-facing inlets allow blood to more readily enter bleed-back channel (364). Distal port (371) of bleed-back tube (370) is preferably located proximal to bleed-back inlets (368), although port (371) could be located coincident with or distal to bleed-back inlets (368).

Inlets (368) are preferably placed at the same location along the longitudinal axis of sheath (76) (the same distance from the sheath distal tip), although inlets (368) can also be staggered such that one or more are located at different positions along the longitudinal (distal-proximal) axis of outer sheath (76). Each inlet (368) can have any desired shape, including but not limited to circular, ovaloid, elliptical, polygonal, slit-like, slot-like, spheroid, etc.

The dimensions, geometry, and spacing of the various elements providing the bleed-back functionality can be varied in relation to each other to achieve the desired amount of blood flow from blood outlet (372) and the desired delay time between exposure to the blood stream and first appearance of a bleed-back signal from outlet (372). For instance, an increase in the distance that the blood must travel from a blood inlet (368) to bleed-back outlet port (372) corresponds to a decrease in the amount of flow that is present at outlet (372) and an increase in delay time. Other factors include the spacing between inner tube (360) and outer sheath (76), the size and number of inlets (368), the diameter of bleed-back tube (370), the position of distal port (371) of bleed-back tube (370) with respect to inlets (368), and the spacing between the introducer inner wall and the outer wall of sheath (76) (for those embodiments where bleed-back is intended to occur when inlets (368) are proximal to the distal tip of the introducer) can all influence the flow rate (or the size of the flow arc) from blood outlet (372). It is preferable to achieve a blood arc in the range of 1-2 centimeters (at maximum pulsation) to maintain a sufficient visual indicator for the medical professional while at the same time minimizing blood loss from the patient.

A multi-lumen extrusion can be used as an alternative to bonding two distinct tubes together. A multi-lumen extrusion has multiple sidewalls (76-1 and 76-2) that can be viewed as analogous to outer sheath (76) and inner tube (360). A multi-lumen extrusion is also one preferred way to allow the creation of multiple channels within outer sheath (76). FIG. 11F depicts an example embodiment of outer sheath (76) fabricated as a three-lumen extrusion where the vacated distal section (366) is no longer present. FIGS. 11G and 11H depict example cross-sections of a multi-lumen extrusion (taken across bleed-back channel (364)) having independent channels. Viewed differently, the multi-lumen extrusion has a reduced volume channel—one that does not extend about the entire circumference of the inner wall (76-2). The septa (379-1, 379-2, 379-3), which separate the various channels, act to improve pushability and prevent kinking. Each septum (379) can be pierced or skived in select locations to increase channel flow. Alternatively, the channel can be blocked using an adhesive, or heat to flow the material, in order to restrict blood entry. Each sidewall (76-1 or 76-2) can be pierced or skived at the proximal end of the delivery system to enable passage of bleed-back tube (370).

FIG. 11G depicts two channels (or lumens) between sidewalls 76-1 and 76-2, an upper channel 364 and a lower channel 376, each being of similar size. Upper channel (364) is the bleed-back channel and has inlets (368) located in positions offset from bleed-back tube (370). The inner-most channel, or lumen, (373) provides a pathway for the proximal portion of foot member (92) (not shown). FIG. 11H is a three-channel extrusion where the upper channel (364) is of reduced volume as compared to FIG. 11G. The non-bleed-back channels (376, 377) can be vacant or used for other functionality (e.g., to accommodate other elongate devices). The reduction in volume of the bleed-back channel is another feature that may be varied to accomplish the desired bleed-back flow characteristics. Referring back to FIG. 11B, it should be noted that a similar configuration can be created with a hybrid single-to-multi lumen extrusion where a outer sheath wall 76 is formed with a single distal extrusion and a dual extrusion on the proximal section forms walls 76-1 and 76-2 (such that the inner extrusion is comparable to inner tube (360). In such an embodiment, the coupling (362) is formed by the Y-junction at the intersection of single to dual extrusion.

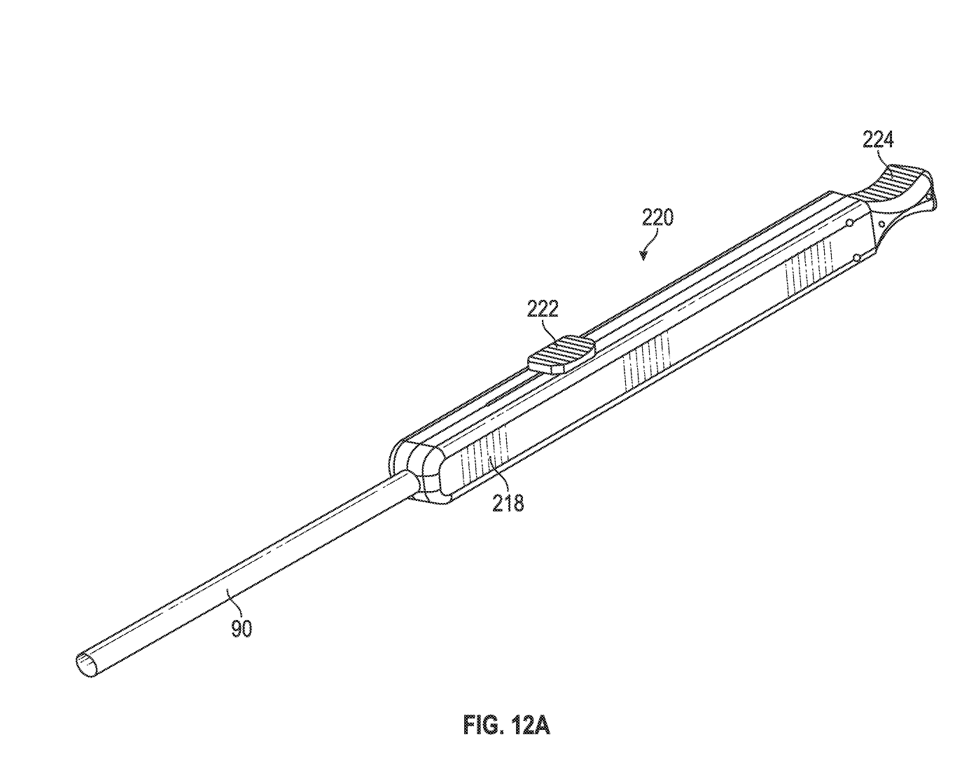

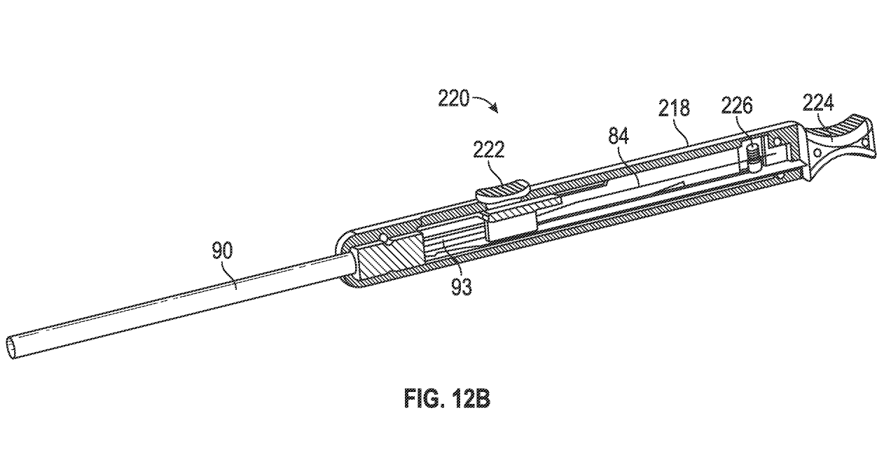

Referring to FIGS. 12A-12D, various aspects of a manual interface or control handle assembly for operating a closure device deployment system, such as those described in reference to FIGS. 6A-6X and 9A-9E, are illustrated. Referring to FIG. 12A, in one embodiment the control handle assembly (220) including a manipulable handle housing (218) and two manually movable elements (222, 224) configured to assist with various aspects of the deployment, is coupled to an elongate deployment member (90). Referring to the partial cutaway view of FIG. 12B, a foot member (93) extends distally through the elongate deployment member (90) and proximally is coupled to the first manually movable element (222), which is slidably coupled to the handle housing (218) to facilitate convenient thumb or finger insertion/retraction of the foot member (93) relative to the elongate deployment member (90). Extending proximally from the foot member (93), an attachment tension member (84) is coupled to a second manually movable element (224) by way of a spring-loaded mechanical fitting (226) configured to provide the operator with a tactile bump in tensile pull (using, for example, a small enlargement in the tension member that passes a fitting within the spring-loaded mechanical fitting) when he is about to release the attachment tension member from the housing, such that it may be left in situ. In operation, when the second movable element (224) is pulled proximally by a small amount (228) as in FIG. 12C, the movable element (224) detaches from the housing. Additional proximal pulling (230), as shown in FIG. 12D, takes the attachment tension member (84) past the detent, bump, or pull limit tactile feedback mechanism, to provide the operator with an understanding that pulling past such point permanently releases the attachment tension member (84) from the housing (84) and the control handle assembly (220) in general.

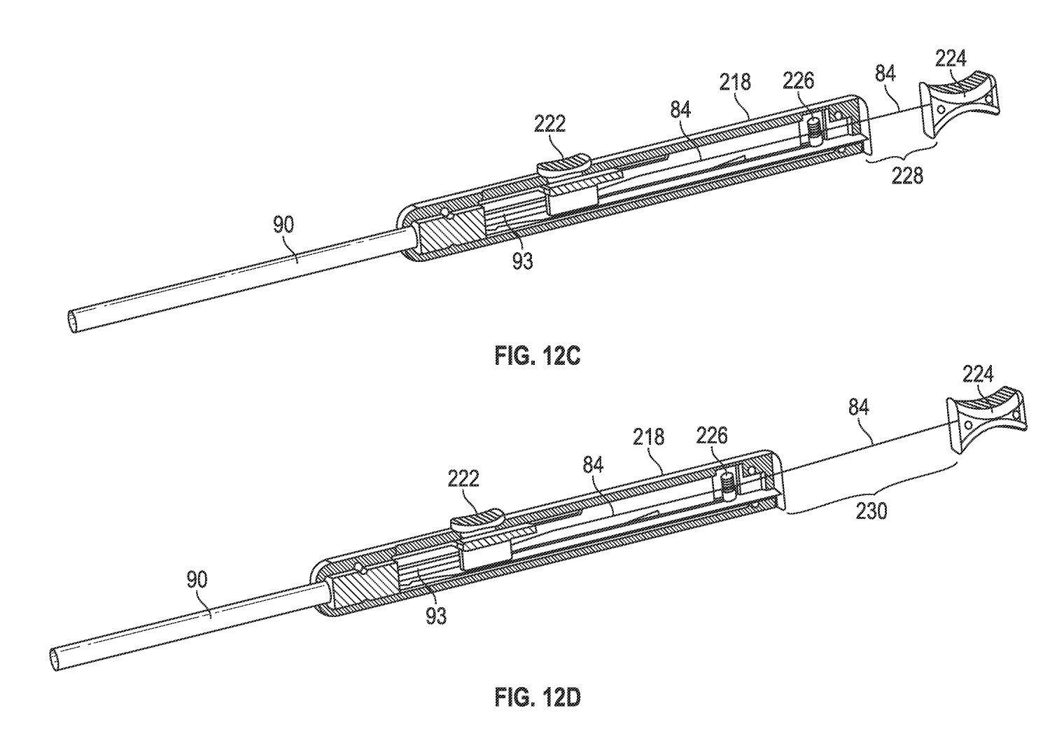

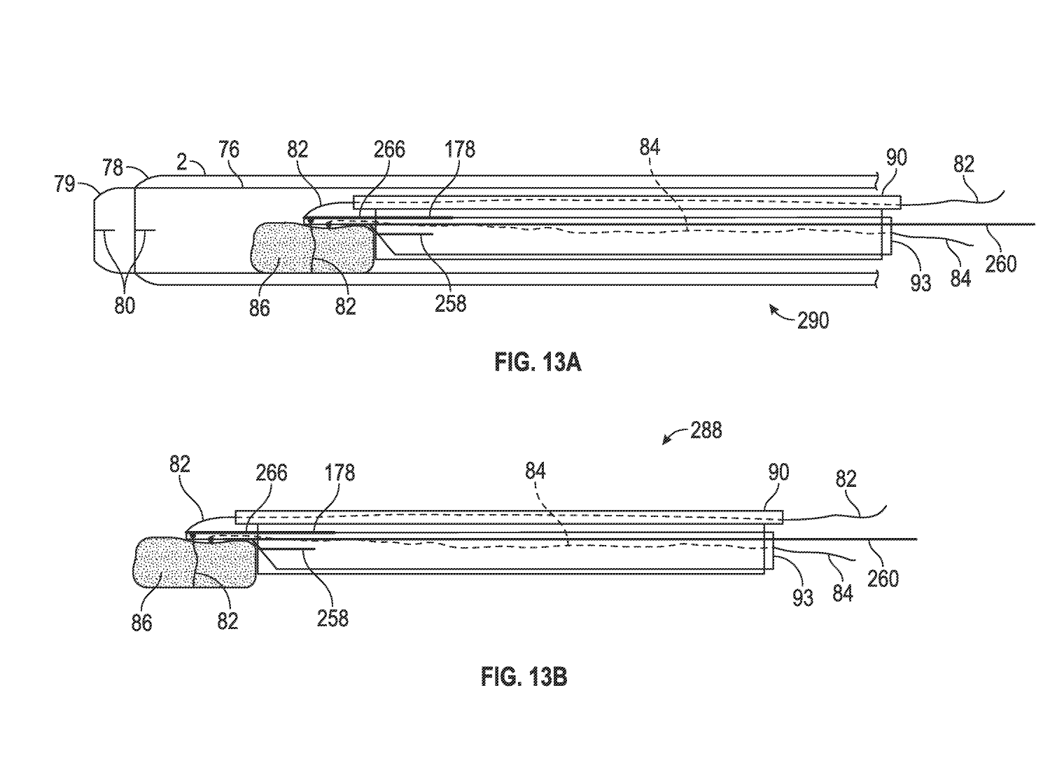

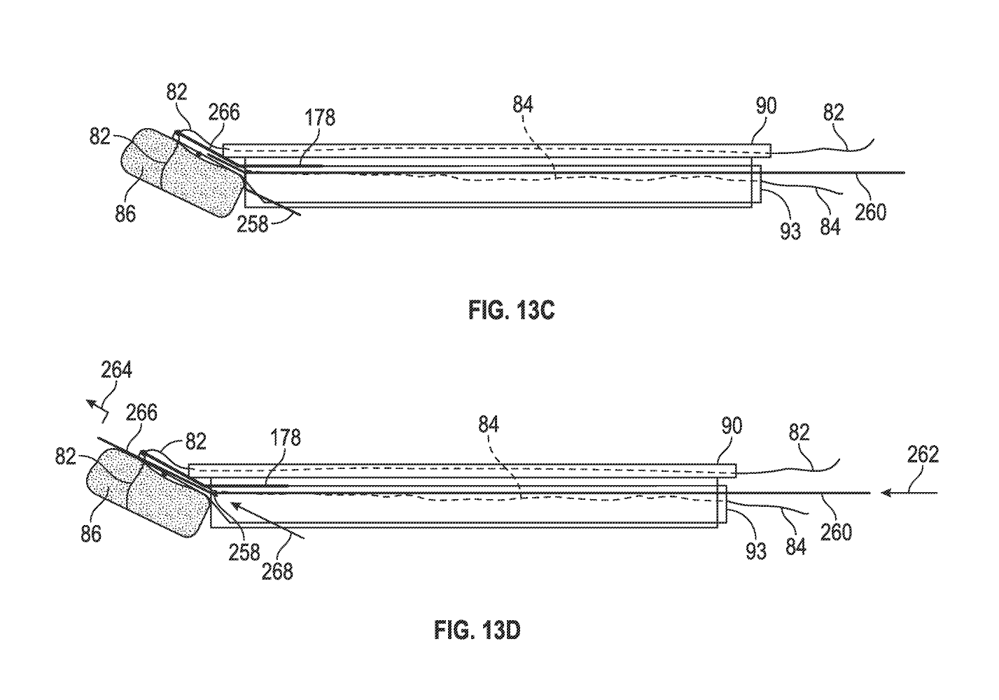

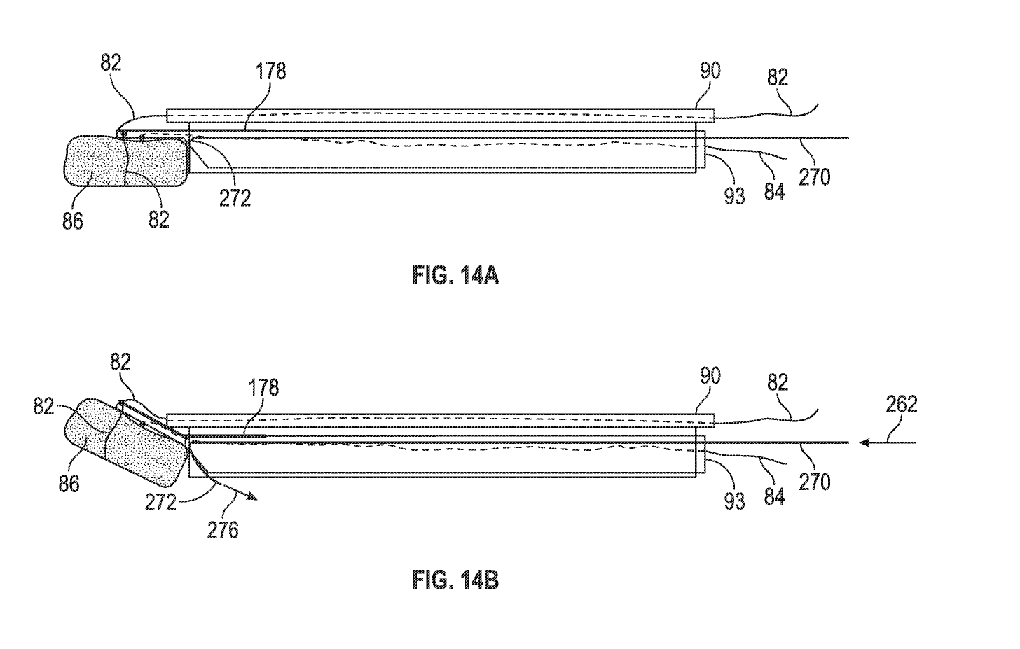

Referring to FIG. 13A, an assembly (290) similar to that of FIG. 9A is depicted, with the exception that a catch member (258) is depicted extending proximally away from the proximal end of the collapsed closure device (86). In the depicted embodiment, the catch member (258) is coupled to an elongate member (260) which has a distal extension (266) that extends distally past the junction of the catch member (258) and the elongate member (260). The assembly (288) of FIG. 13B has similar elements as shown in FIG. 13A, with the exception that the introducer sheath (2) and delivery sheath (76) have been removed. Referring to FIG. 13C, when the distal aspect of the foot member (93) is moved, as described above in reference to FIG. 9B, for example, the catch member (258) also becomes reoriented, to a configuration wherein the proximal aspect of the catch member sticks out, away from the remaining portions of the delivery assembly, to facilitate “catching” tissue structures which may be nearby, such as the tissue which may surround a hole or defect. This “catching” affect desirably prevents the associated closure device (86) from exiting such defect or hole, as described further below. Further, this catching affect assists with docking of the collapsed state of the implantable closure device adjacent the arteriotomy, and may also be useful in mechanically supporting the portions of the vessel that lie immediately adjacent the catch member (i.e., in one configuration, the catch member may be utilized to prevent collapse of the immediately adjacent vessel portions). Referring to FIG. 13D, in one embodiment, the catch member (258) may be controllably retracted forward (268) with advancement (262) of the elongate push/pull member (260) through manual manipulation. Such advancement of the elongate member (260) causes advancement (264) of the distal extension (266), which guides the catch member (258) forward and into alignment with the distal extension (266), into a refracted position as shown in FIG. 13D. Such retracted position may be utilized to retract a delivery assembly back through the hole or defect after deployment of a closure device. Referring to FIGS. 14A and 14B, a similar catch member functionality may be achieved in one embodiment by advancing (274) and/or retracting a guidewire (270) through a lumen created in the foot member (93) to cause a guidewire distal tip portion (272) to be advanced outward (276) relative to the collapsed closure device (86) in a “catching” configuration, or retracted back to facilitate removal of the assembly through the hole or defect.

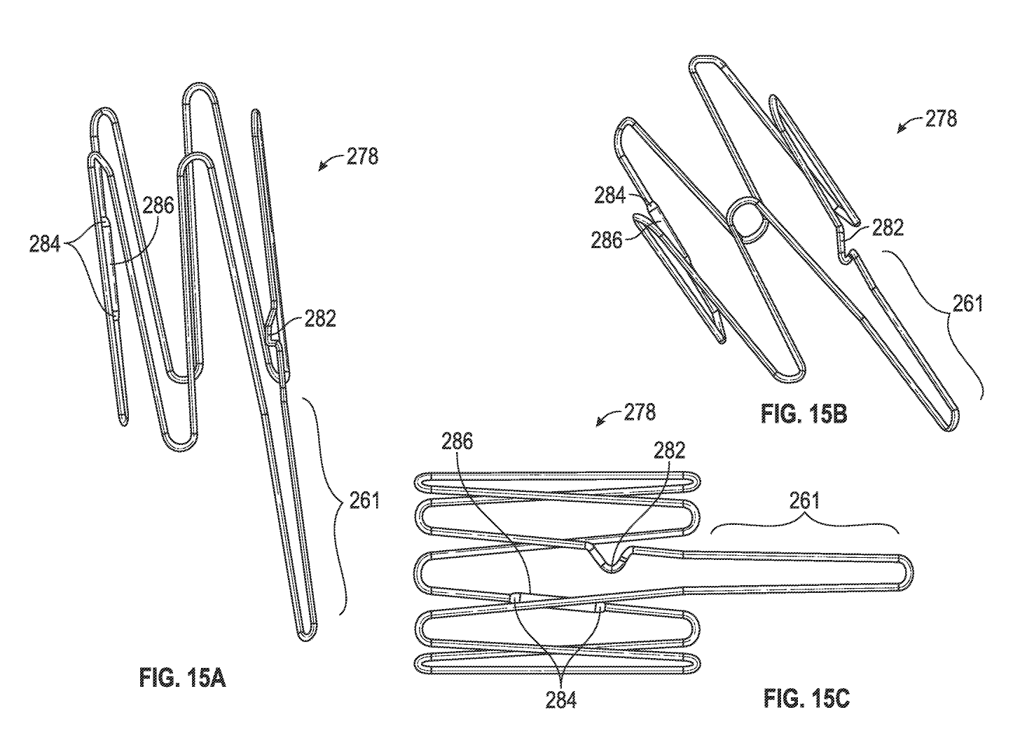

Referring to the three different orthogonal views of FIGS. 15A-15C, a catch member extension (261) may include a portion of the closure device scaffold (278), as opposed to a separate movable member as described above in reference to FIGS. 13A-14B. Such a catch member extension (261) is not configured to be retractable, but need not be, as it is decouplable from the delivery tools, and such tools may therefore be retracted away through the hole or defect without concerns for reconfiguring the catch member for such retraction (i.e., because in this embodiment, the catch member need not be withdrawn back through the hole or defect after closure device deployment). Also shown in FIGS. 15A-15C are small omega formations (282) which may be utilized for fastening tethers, a cover, and the like, as described above in reference to FIGS. 6W and 6X. Further, a wire coupling is shown, including a sleeve (286) that is fitted over to abutted ends of the wire comprising the depicted scaffold (278) and welded (284) in place to form a robust junction by melting the sleeve over the wire ends. In one embodiment, the inner diameter of the sleeve may be about ½ of a thousandth of an inch larger than the outer diameter of the wire, for a tight fit, before the sleeve is melted (notwithstanding the fact that in one embodiment, both the wires and sleeve comprise NiTi material, preferably the wires remain unmelted and as intact as possible), forming a low-profile filleted joint which may be electropolished for smoothing before attachment of a cover member, tether, or other part. In other embodiments, such a junction may be created using adhesive bonding, crimping, press fit techniques, and/or thermal expansion/contraction techniques.

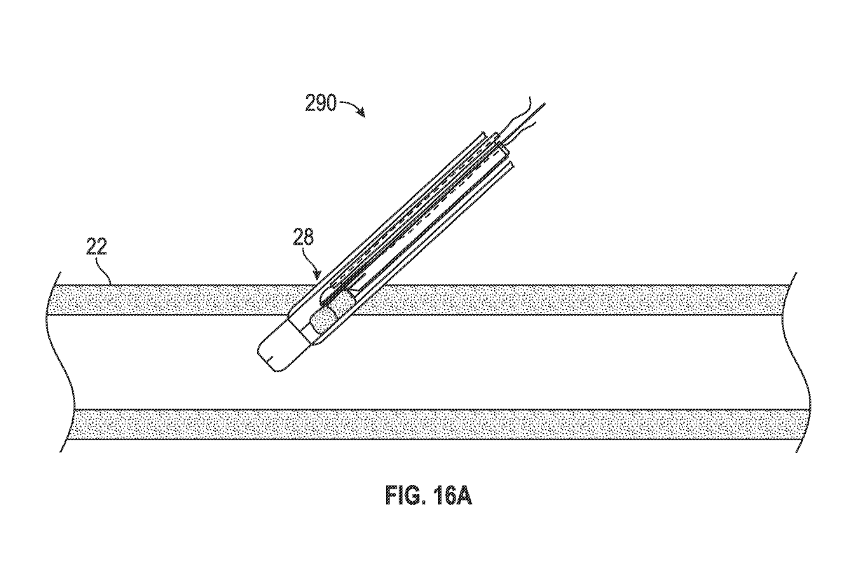

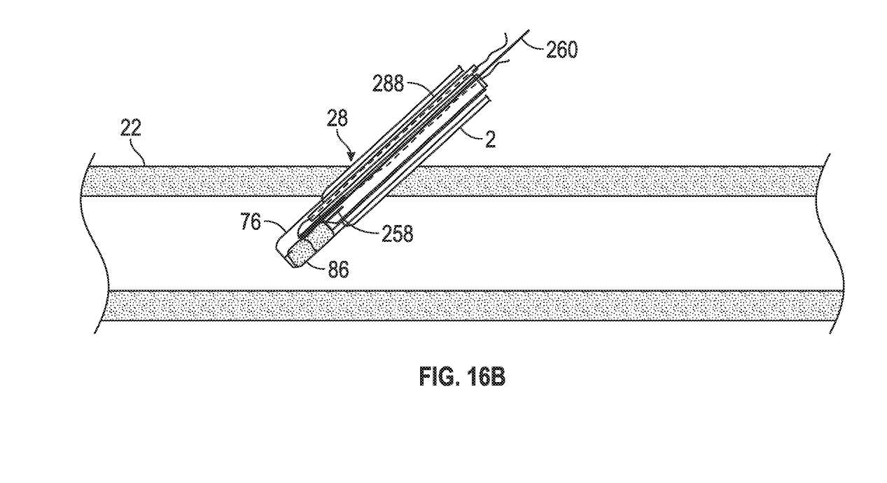

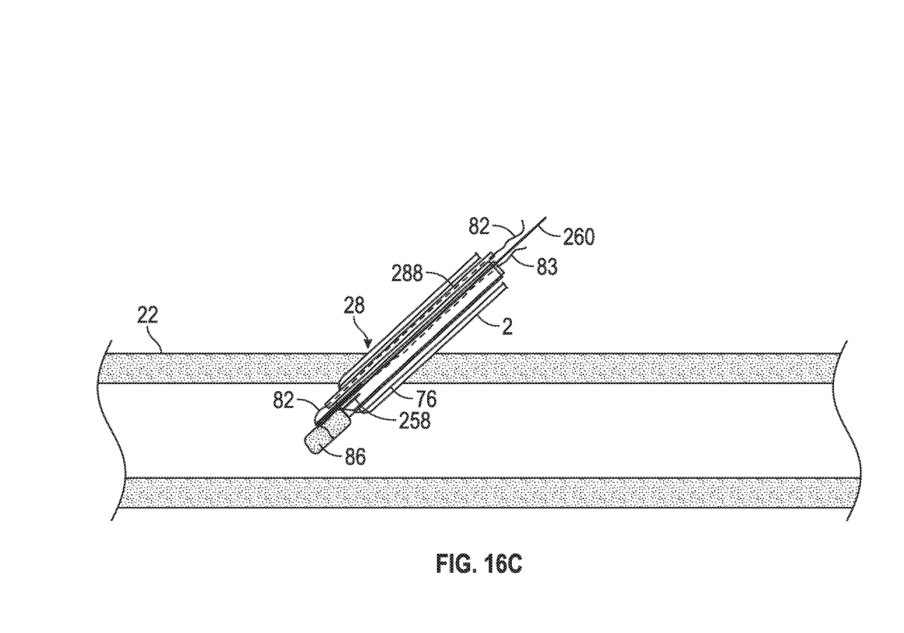

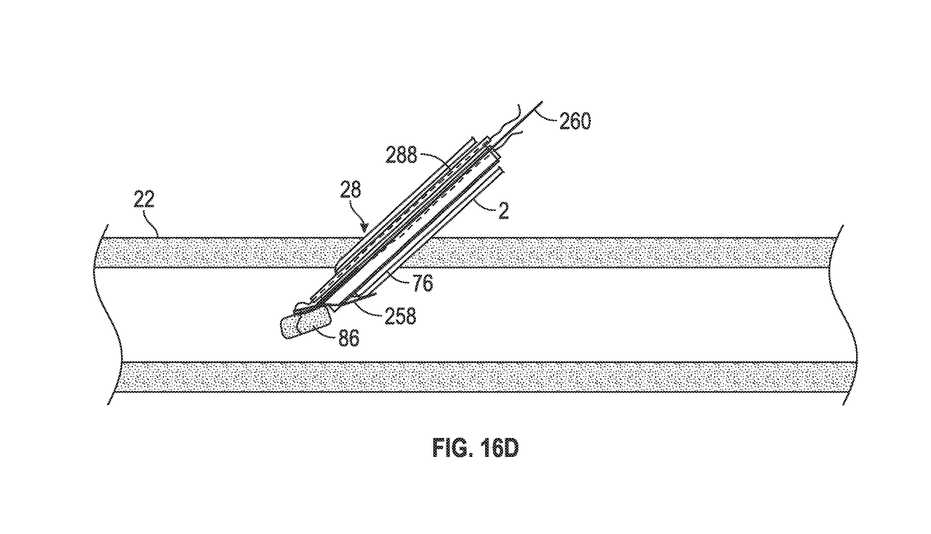

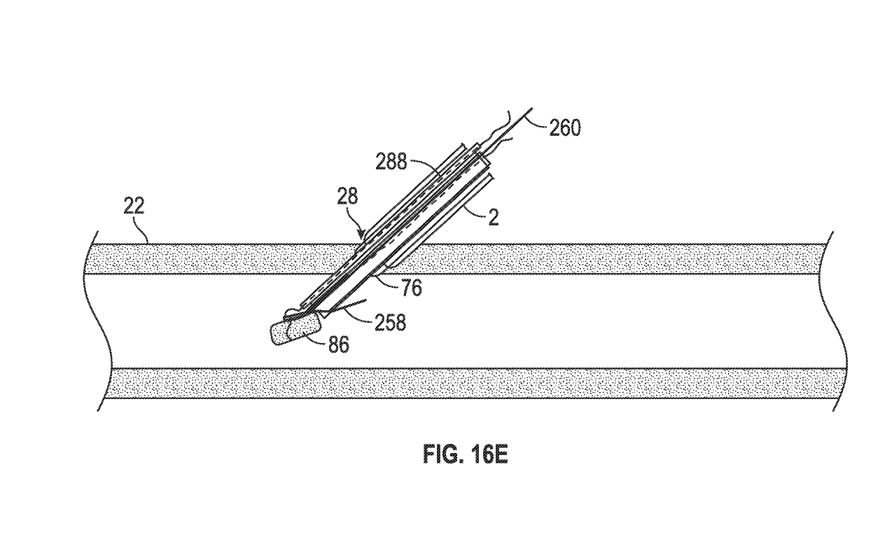

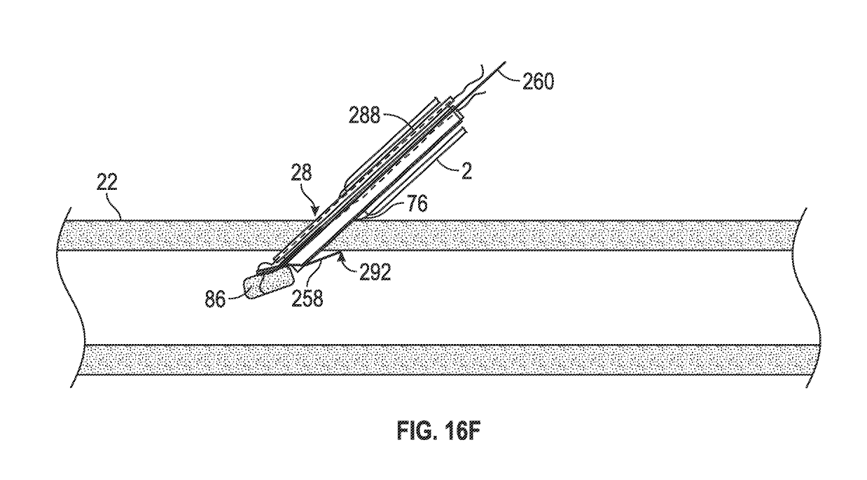

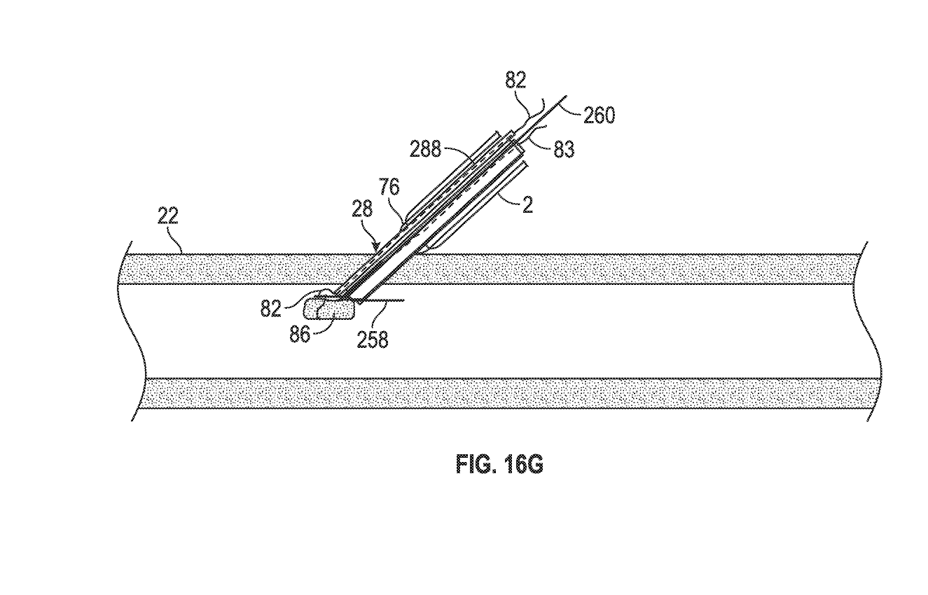

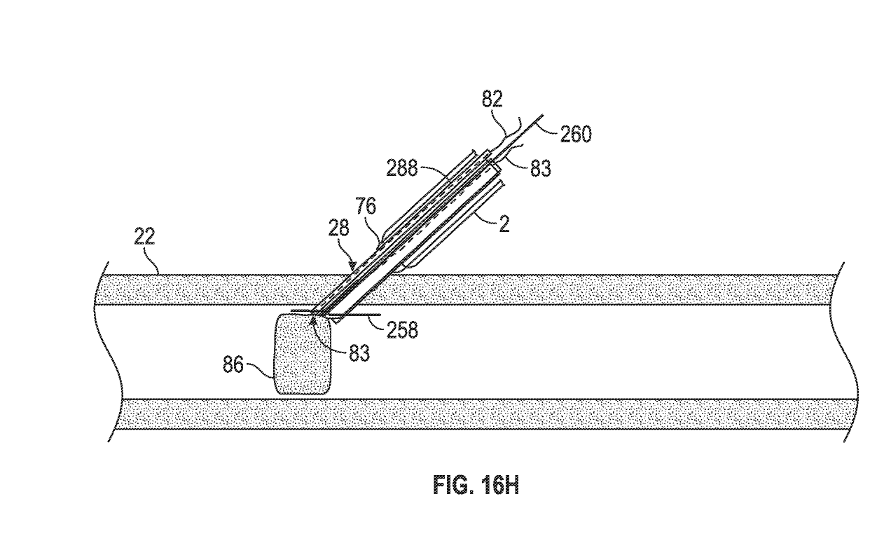

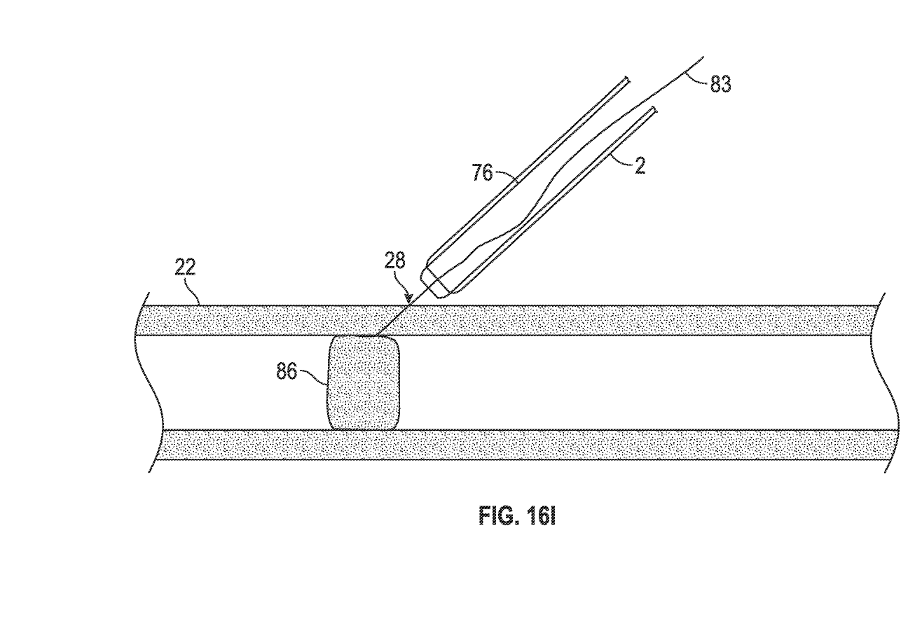

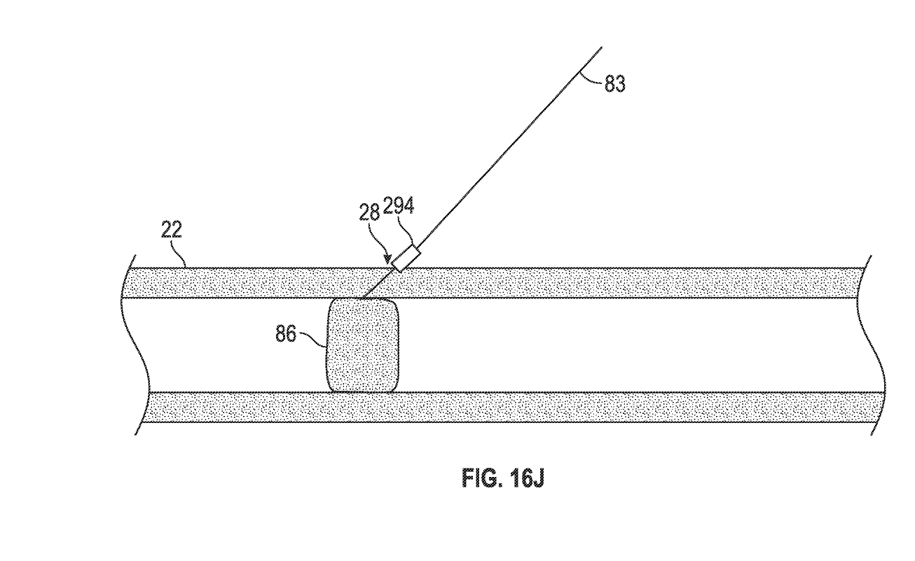

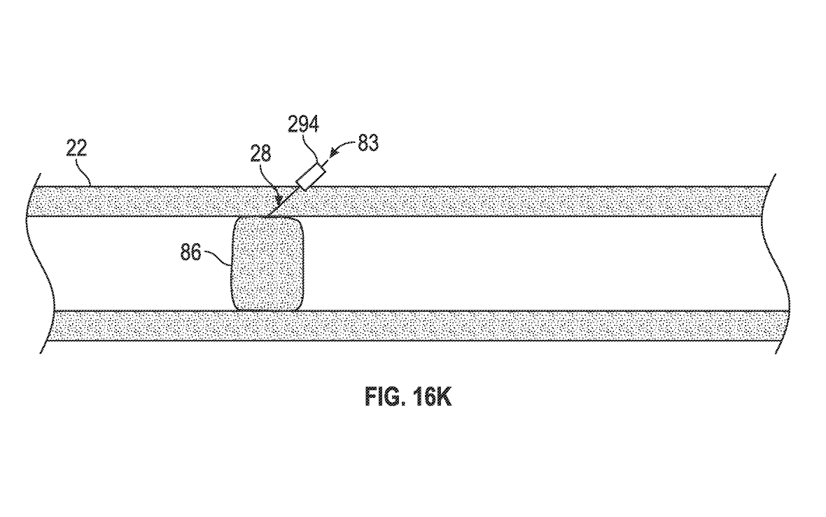

Referring to FIGS. 16A-16K, various aspects of a deployment are configured utilizing embodiments similar to those described in reference to FIGS. 13A-13D above. Referring to FIG. 16A, an assembly (290) similar to that of FIG. 13A is depicted being inserted through an arteriotomy (28) formed in a vessel (22). Referring to FIG. 16B, the delivery sheath (76) and collapsed closure device (86) may be advanced relative to the introducer sheath (2) to expose the delivery sheath (76) and collapsed closure device (86) to the bloodstream of the vessel (22). The delivery sheath (76) may include a channel or lumen for providing bleed-back feedback to the operator, as described in reference to FIG. 11, such that the operator may insert and/or withdraw the delivery sheath (76) and collapsed closure device (86) until a desirable predetermined level of insertion is achieved, based upon the feedback from the bleed-back configuration. Referring to FIG. 16C, with the delivery sheath (76) and collapsed closure device (86) optimally inserted relative to the arteriotomy (28), the delivery sheath (76) may be controllably withdrawn while the longitudinal position of the collapsed closure device (86) is maintained, and as shown in FIG. 16D, the foot member may be utilized to induce repositioning (in the depicted embodiment, rotational repositioning or reorienting) of both the collapsed closure device (86) and the catch member (258). Referring to FIGS. 16E and 16F, the introducer sheath (2) and delivery sheath (78) may be further withdrawn, and the collapsed closure device (86) and catch member (258) may be withdrawn or urged toward the tissue (292) surrounding the arteriotomy. As shown in FIG. 16F, the catch member interfaces with the nearby tissue (292) and geometrically prevents the closure device (86) from being withdrawn out of the arteriotomy (28). With the collapsed closure device (86) appropriately positioned and oriented prior to expansion of the closure device to an expanded configuration, the closure device may be expanded to the expanded configuration (88), as shown in the transformation from FIG. 16G to FIG. 16H. Referring to FIG. 16I, with the closure device (88) deployed into the expanded configuration, the elongate deployment member (90), foot member (93), and catch member (258) and associated elongate member (260) may be withdrawn proximally, preferably after the elongate member (260) has been advanced relative to the foot member (93) to place the catch member into a withdrawn configuration, as described above. As shown in FIG. 16J, the introducer sheath (2) and delivery sheath (76) may also be withdrawn, leaving behind the attachment tensile member (83) which is coupled to the closure device (88). Referring to FIG. 16K, in one embodiment, a resorbable compressive member (294), such as a sheet or cylindrical plug member (294) of collagen or other bioresorbable material slidably coupled to the attachment tensile member (83), may be advanced toward the arteriotomy to place the arteriotomy wound in compression between the member (294) and the deployed closure device (88). The surrounding tissue would serve to maintain the position of the member (294) relative to the arteriotomy, but in one embodiment, one or more sutures may be utilized to fasten the member (294) in position relative to the arteriotomy (28). In another embodiment, a flowable material, such as hydogel or other pro-thrombal agent selected to promote healing and/or clotting, may be dispensed adjacent the arteriotomy.

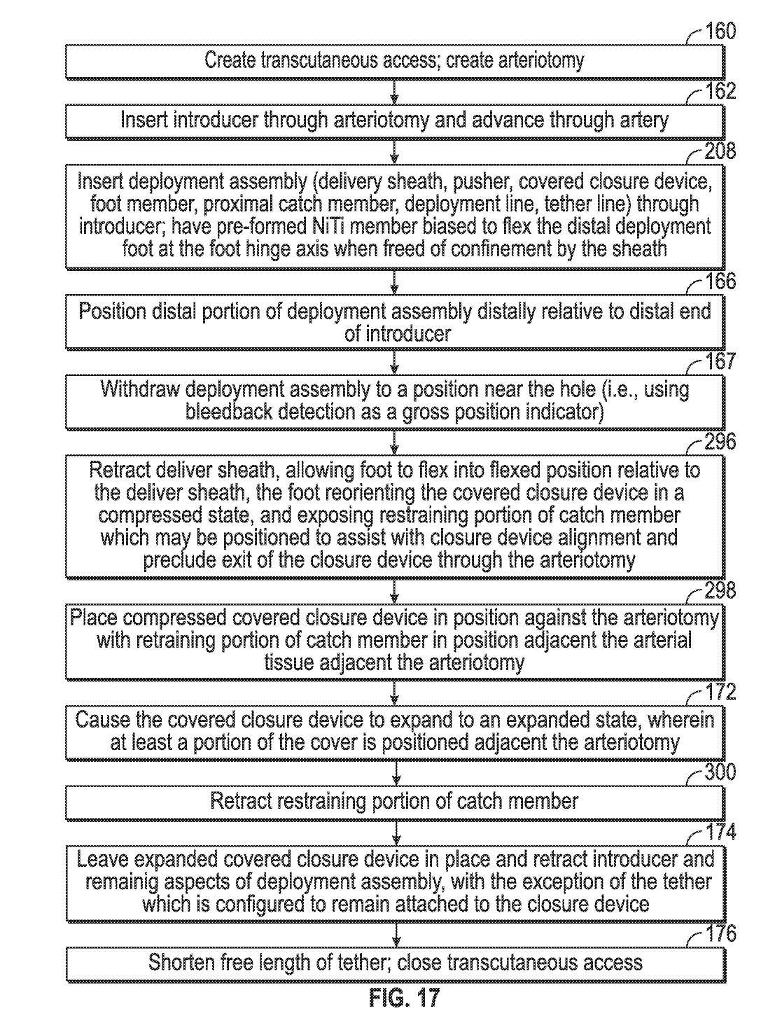

Referring to FIG. 17, a process embodiment is illustrated featuring configurations such as those described above in reference to FIGS. 16A-16K. Referring to FIG. 17, with several steps in common with the process embodiments described in reference to FIGS. 8A, 8B, and 10, after transcutaneous access and arteriotomy are created (160) and introducer advancement (162), a deployment assembly comprising a collapsed closure device movably coupled to a proximal catch member and other tools may be inserted through the introducer (208), positioned relative to the end of the introducer (166), and adjusted longitudinally to a position near the arteriotomy (for example, using bleed-back detection to confirm longitudinal positioning relative to a predetermined position associated with bleed-back, as described above in reference to FIG. 11; 167). The foot may be controllably flexed to reorient and/or reposition the collapsed closure device and catch member (296), after which the collapsed closure device and catch member may be urged into a position adjacent and/or against the arteriotomy, with the catch member in position to prevent withdrawal or escape of the closure device through the arteriotomy (298). Subsequently the closure device may be expanded to the expanded configuration (172), the catch member may be refracted to a withdrawal configuration (300), other deployment tools may be retracted/withdrawn (174), and the free length of tether remaining may be shorted before transcutaneous access is closed.

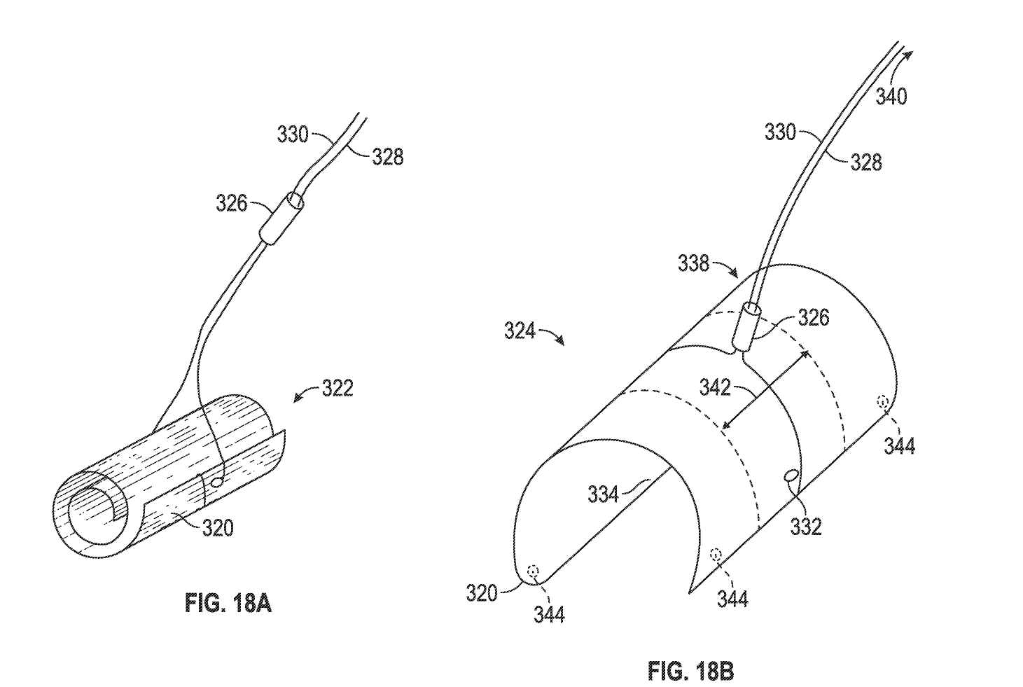

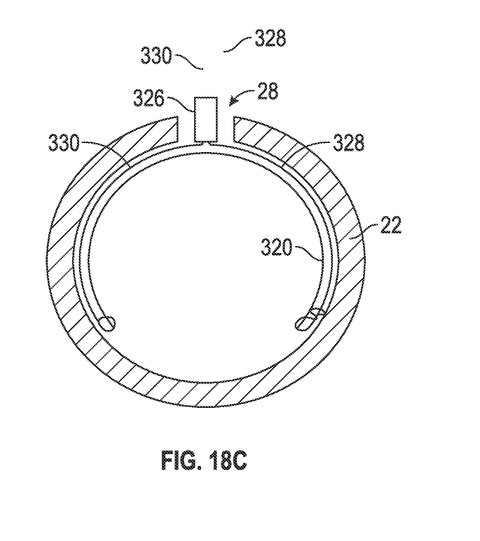

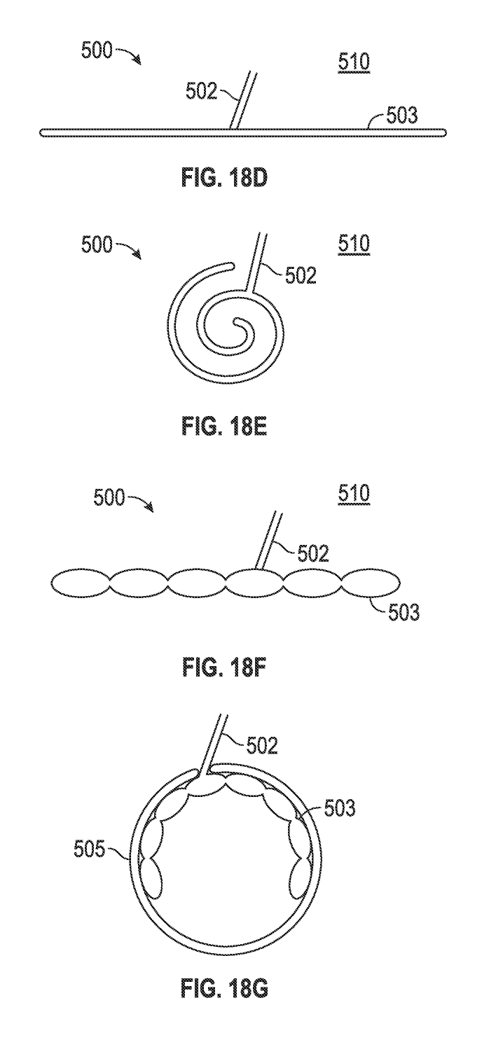

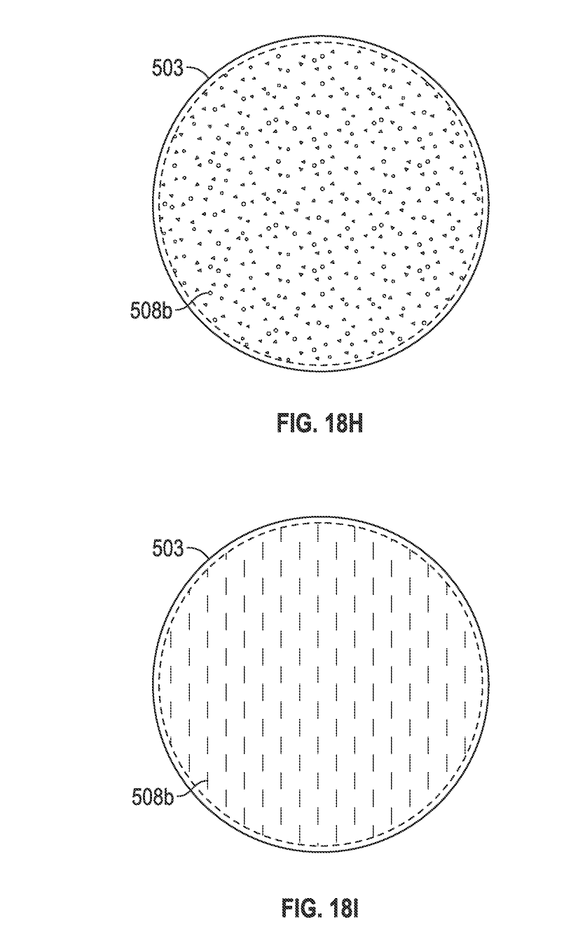

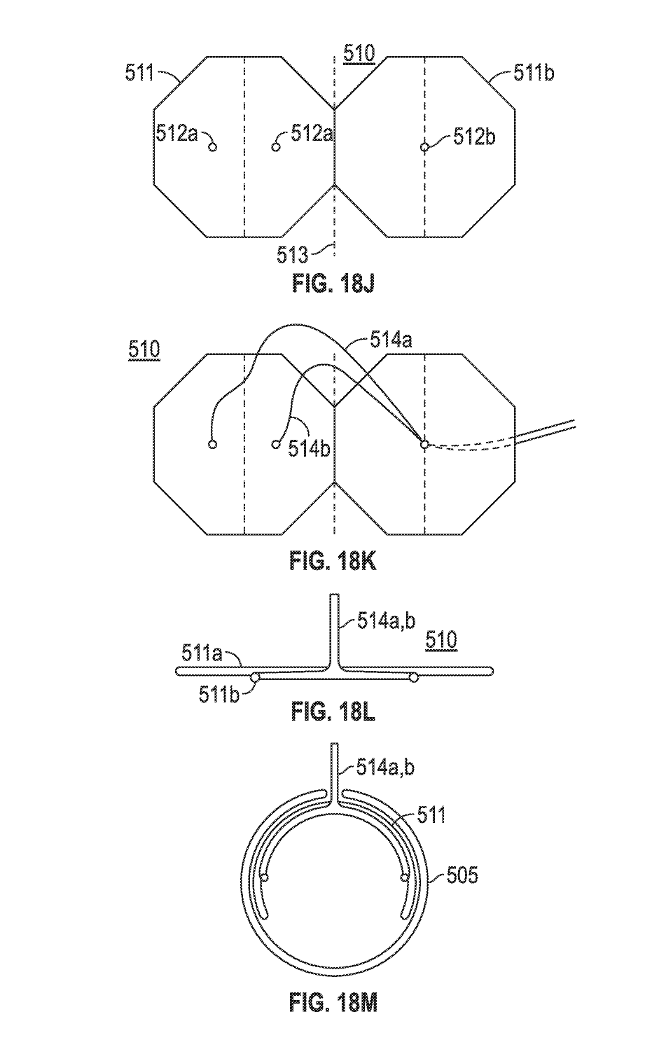

Referring to FIGS. 18A-18C, a fully bioresorbable closure device configuration is depicted. Referring to FIG. 18A, in a collapsed configuration (322), a “furling”/“unfurling” closure device (320) in the “furled” or collapsed state has a relatively small outer diameter. Referring to FIG. 18B, with tension on the one or more tensioning elements (328, 330), which may be coupled to the depicted coupling points (332, 334), or in another embodiment featuring four tensioning elements (not shown), to the other depicted coupling points (344—four corners of the substantially rectangular sheetlike closure device), and concomitant pushing of a cinching member (326) toward the closure device (i.e., to create a compressive load 338 between the cinching member 326 and closure device 324), the closure device assumes an expanded configuration (324) wherein a substantially cylindrical shape is assumed, at least in part. In the depicted embodiment, the arc length occupied by the expanded closure device (324) represents approximately ⅔ of the circumference of a full cylindrical shape. In another embodiment, the expanded closure device (324) represents approximately ½ of the circumference of a full cylindrical shape. The cinching member (326) may include a housing through which the one or more tensioning elements are passed, and may include a locking feature, such as a simple reed lock (i.e., as in a “zip tie” locking mechanism) to allow only tightening movement. The middle portion (342) adjacent the tensioning member portions coupling to the closure device (324) may be reinforced to prevent bucking under the applied loads—to facilitate controllable expansion with applied tensioning (340) and compression (338) of the cinching member (326). Referring to FIG. 18C, a side view of a deployed configuration such as that illustrated in FIG. 18B is depicted, showing the expanded closure device configuration (324) occluding the arteriotomy (28).

In another embodiment similar to that illustrated in FIGS. 18A-18C, one or more portions of such embodiment may be nonresorbable. For example, in one embodiment, a sheetlike portion of the unfurling prosthesis (320) may be bioresorbable, while one or more structural members comprising materials such as one or more nonresorbable metals or polymers may be coupled to the sheetlike portion to assist with the unfurling or other deployment steps or configurations.

FIGS. 18D-18G depict additional example embodiments of an inflatable closure device that may include a two-layer bioresorbable material are depicted. Although two-layer (or layers) are depicted, one of ordinary skill could just as readily use a plurality of layers without deviating from the teachings described herein. Referring to FIG. 18D, a deflated two-layer closure device (503) is depicted in a flat configuration with an inflation member (502) extending therefrom. The inflation member (502) is used to inflate the two-layer closure device (503) using any inflation medium, including, for example, a hydrogel, water, or air. Referring to FIG. 18E, the deflated two-layer closure device (503) may be rolled or furled into a collapsed state, resulting in a relatively small outer diameter. Referring to FIG. 18F, once an inflation medium is injected into the two-layer closure device (503), the inflation medium fills the void sandwiched between the two-layer closure device (503), thus causing expansion or pillowing of the two-layer closure device (503). The inflation member (502) may also double as a tension member so as to keep the two-layer closure device taut adjacent to the vessel (505). Referring to FIG. 18G, an inflated two-layer closure device (503) is shown in its expanded state within the vessel (505) with the inflation member (502) extending from within the vessel (505), through the arteriotomy, and beyond the exterior of the vessel (505). Once the two-layer closure device has been inflated within the vessel (505), the two-layer closure device (503) assumes an expanded or inflated configuration that has the shape of a partial tube. It should be noted that a fully tubular shape could be used, that extends around the entire circumference of the vessel, or a fraction thereof sufficient to occlude the arteriotomy, leaving only the inflation member (502) exposed therefrom. In the depicted embodiment, the arc length occupied by the inflated two-layer closure device (503) represents approximately ⅔ of the circumference of a full cylindrical shape. In another embodiment, the inflated two-layer closure device (503) occupies approximately ½ of the circumference of the vessel (505). As is also evident when comparing FIG. 18G with 18E, the inflation of the two-layer closure device (503), will naturally expand the closure device (503) from a collapsed state to an expanded state, that is, an unrolling or unfurling transformation. It should be noted that the closure device (503) does not have to overlap itself in the rolled or furled configuration. Instead, this expansion can be broadly viewed as a transformation from a curled or bunched state to a relatively more linear state (when viewed end-on as in FIG. 18G).

Referring to FIGS. 18H-18I, a top-down view of a two-layer closure device in an unfurled configuration is depicted. Each of the two-layer closure devices (503) includes two-layer bioresorbable material that is coupled together at one or more locations, such as with adhesive, mechanical connectors, dot welds, slot welds, and the like. As with any bioresorbable material, the material will gradually resorb and be benignly cleared from the body, leaving no permanent implant within the vessel. Thus, special considerations should be placed on choosing the correct weld placement onto the two-layer closure device (503). Referring to FIG. 18H, two individual circular (or generally circular)-shaped bioresorbable sheets are placed on top of one another. The circumference is then hermetically sealed, thereby creating a two-layer closure device (503). To prevent the two layers from ballooning apart into a cross-sectional football shape that occludes the flow of blood, the two layers are preferably connected to limit the amount of ballooning or expansion. This can be done with one or more connections or junctions. In the embodiment of FIG. 18H, this is accomplished by a plurality of discrete point-like connections, e.g., weld spots that can be distributed relatively evenly across the portions of the layers that will be inflated. Alternatively, these individual junctions may also be weld slots (508b), as shown in the embodiment illustrated by FIG. 18I. Accordingly, upon insertion of an inflation medium (not shown) into the sealed portion of the two-layer closure device (503), a cross section of the device (503) may look like that depicted in FIGS. 18F and 18G. Notably, although the two-layer closure device is depicted as having a circular shape, one of ordinary skill in the art may appreciate that any shape may be used in the alternative without substantially deviating from the novel aspects of the present disclosure, e.g., elliptical, square, rectangular, polygonal (with identical or varying edge lengths and with sharp or rounded edges).

In an alternative embodiment, the two-layer closure device (503) may additionally have a plurality of micro-holes on one or both of the layers. With micro-holes in place, the inside of the two-layer closure device (503) may be exposed to fluid, e.g., blood. A liquid absorbable material may be in place in between the layers with micro-holes, thus, causing expansion of the inside material, which would subsequently transform the two-layer closure device (503) into to the pillowing arrangement as depicted in FIGS. 18F and 18G. Although it may be preferable to use a hydrogel material in between the layers, a person of ordinary skill may choose to use any similar material that expands within the encapsulated layers when exposed to liquid, for example, a hydrophilic, spongy, woven, fiber matrix material that responds to liquid may be suitable.

Referring to FIGS. 18J-18M, various aspects of a two-layer closure device configuration wherein each structure thereof may include a bioresorbable material are depicted. Referring to FIG. 18J, a two-layer polygonal closure device (510) is depicted, wherein one layer (511a) has two apertures (512a) that are offset from a third aperture (512b) centrally placed on the second layer (511b). The apertures (512a/512b) are properly aligned once the layers (511a/511b) are folded upon one another along axis (513), forming a single crease between the first and second layer (511a/511b). Proper routing of sutures (514) is depicted in FIG. 18K. One suture (514a) is terminated through aperture (512a) and the other suture (514b) is terminated through a different aperture (512a). The sutures may be terminated by using a knot, adhesive, clamp, or any other method of permanent or temporary bonding between suture and aperture. Both sutures (514a/514b) are subsequently routed through the third aperture (512b). Once the two layers (511a/511b) are folded together, the sutures (514a/514b) can be adjusted or pulled so as to remove any slack that may exist between the two-layer closure device (510). Referring to FIG. 18L, a cross section of a two-layer closure device configuration is depicted wherein each layer (511a/511b) is substantially in contact with the opposing layer with the sutures (514a/514b) routed therebetween and out the center or third aperture (512b). As described above with respect to FIGS. 18A-18C, the two-layer closure device (510) may be fully bioresorbable and capable of being manipulated into a collapsed state by furling the closure device (510). See FIGS. 18A and 18E. Once in a collapsed state, the two-layer closure device may be manipulated and housed within a sheath for subsequent deployment within an arterial wall as detailed herein.

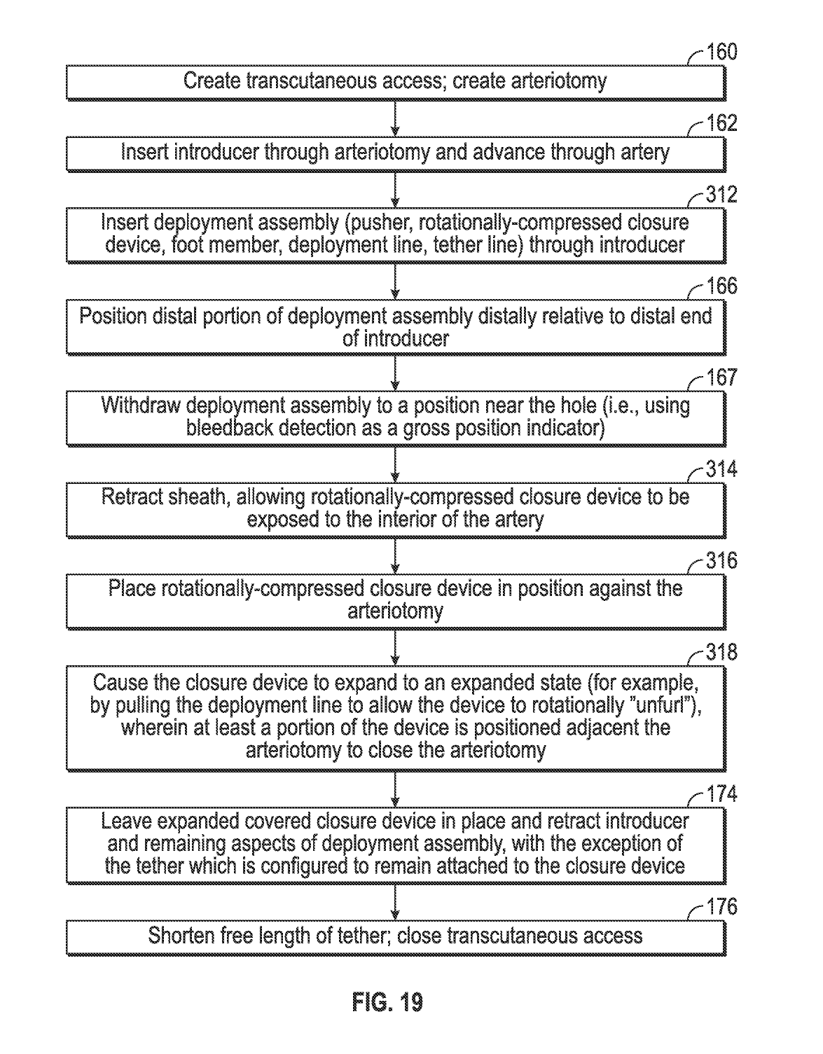

Referring to FIG. 19, a process for utilizing a configuration such as that described in reference to FIGS. 18A-18C is illustrated. Referring to FIG. 19, after transcutaneous access and arteriotomy creation (160) and introducer advancement (162), a deployment assembly comprising a rotationally-compressed, or “furled”, closure device may be advanced into position through the introducer (312). The distal portion of the deployment assembly may be positioned relative to the introducer (166), and bleed-back may be utilized to assist with positioning relative to the arteriotomy (167) before retracting the delivery sheath and allowing the rotationally-compressed closure device to have direct access to the interior of the artery (314), adjust in position relative to the arteriotomy (316), expand or “unfurl” to close the arteriotomy (318)—i.e., by applying tension to associated tension elements, followed by removal of associated tools (174) and final tether shortening and wound closure (176).

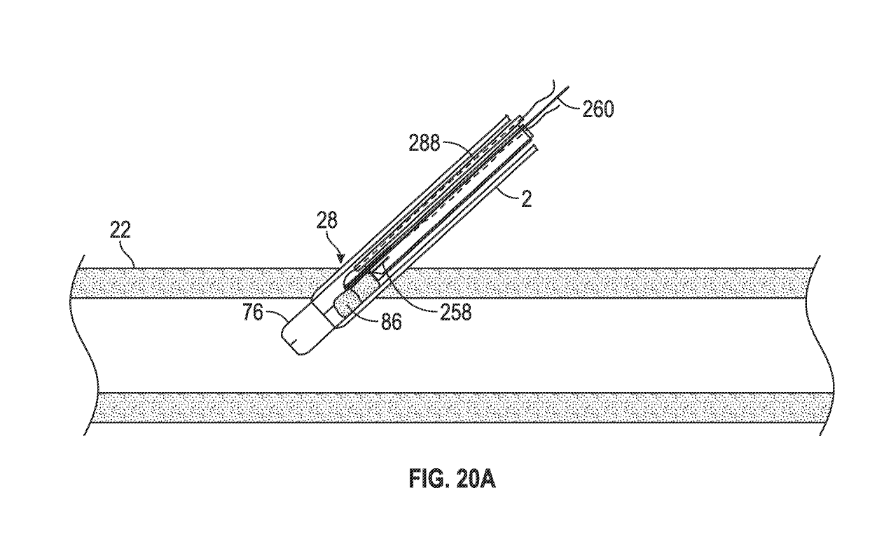

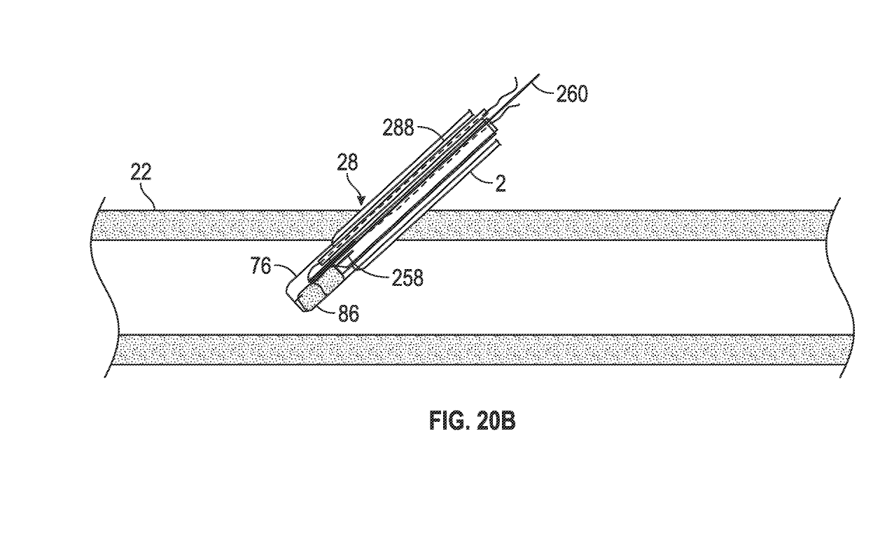

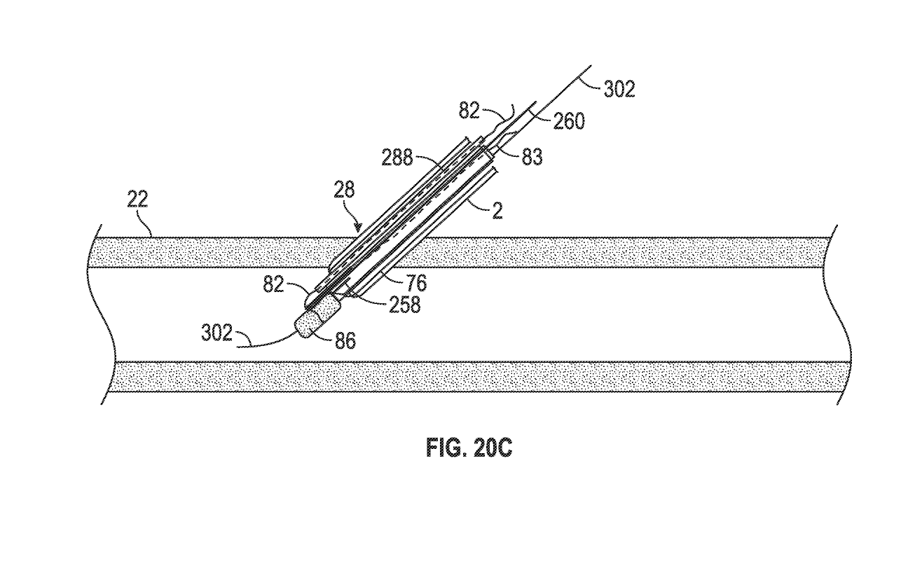

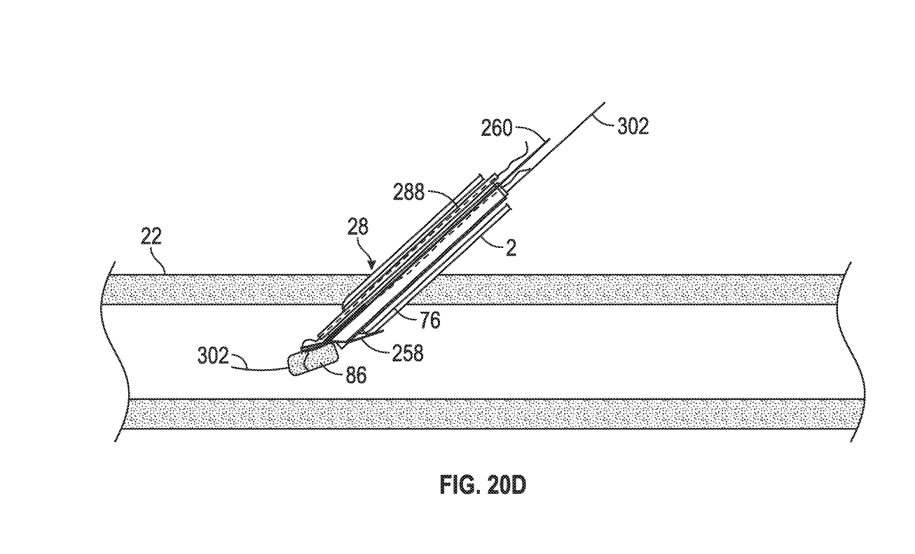

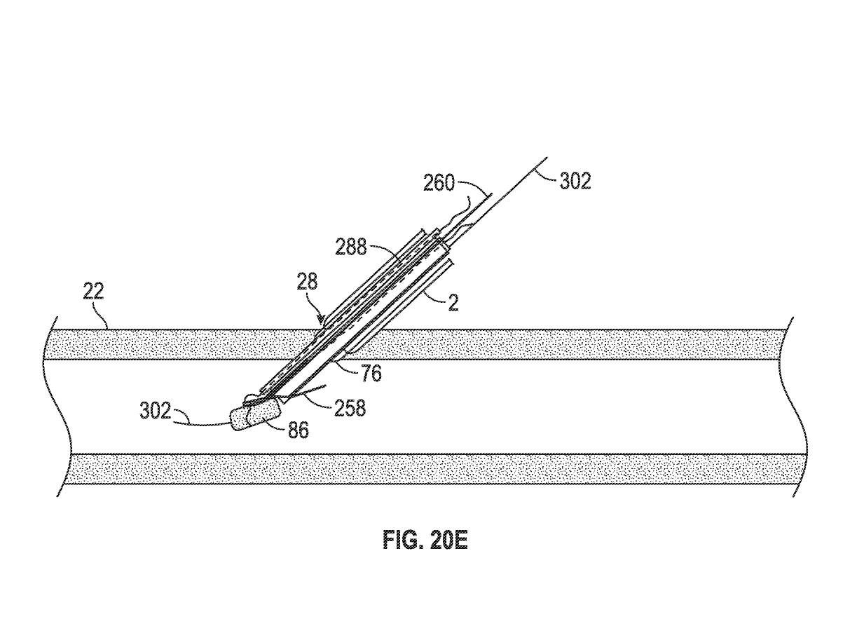

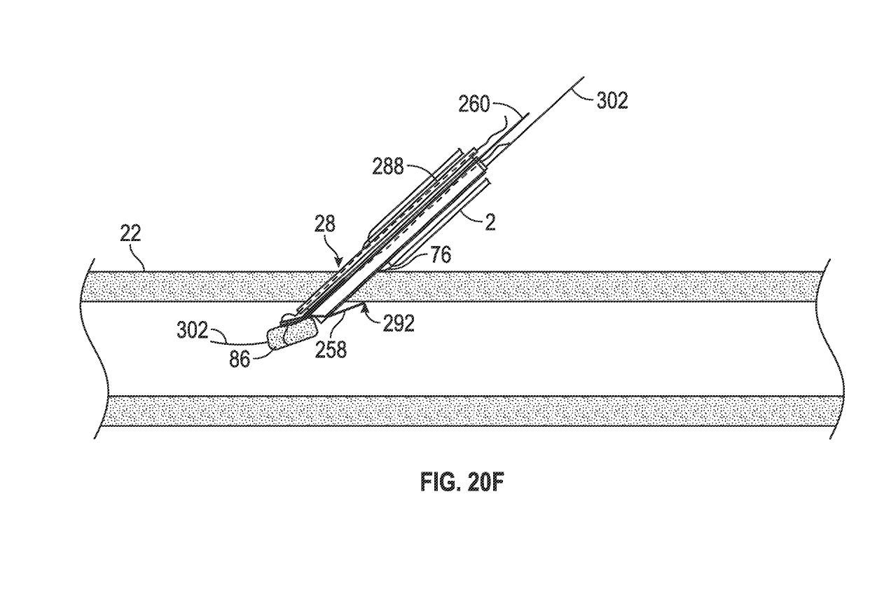

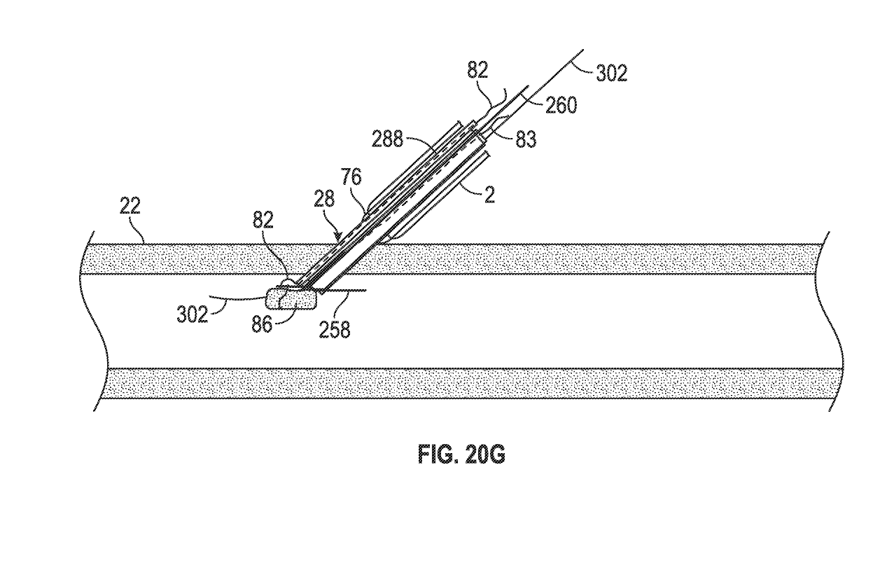

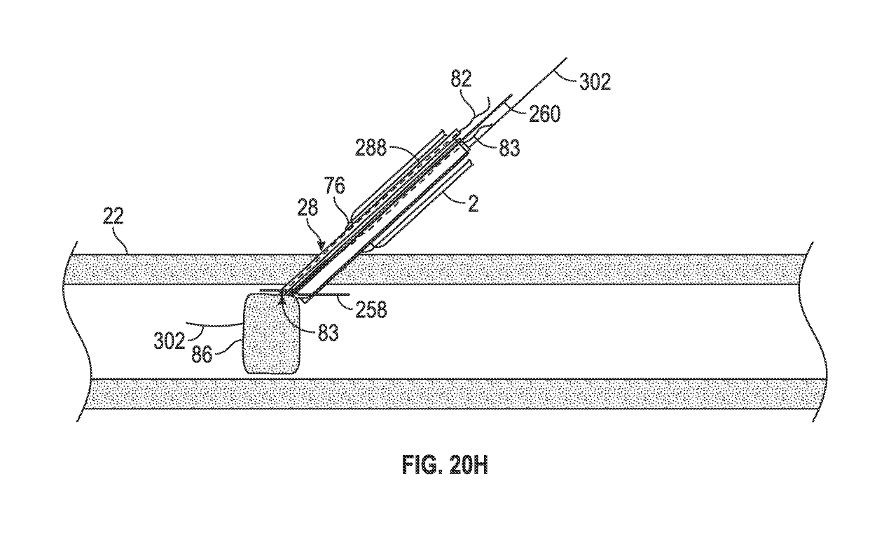

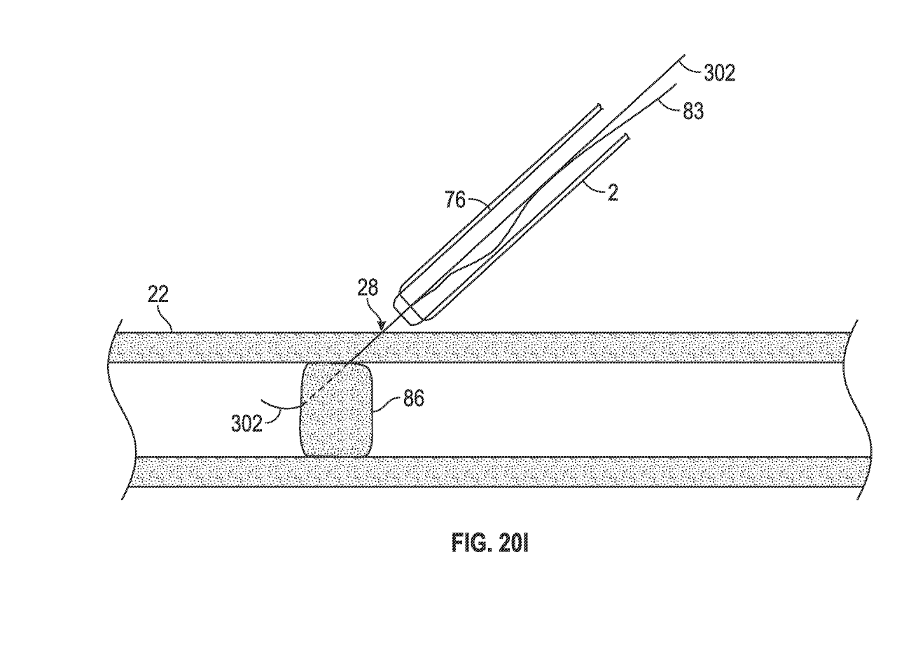

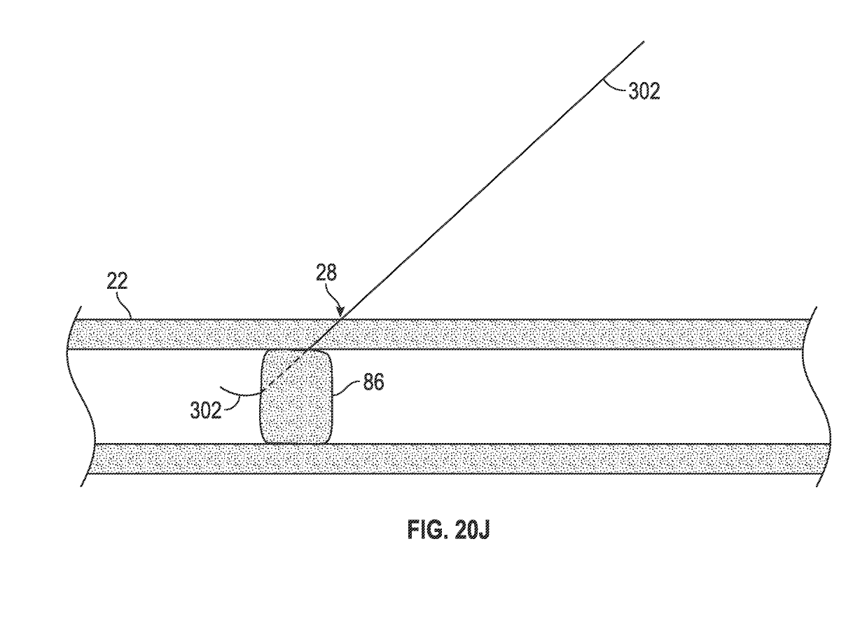

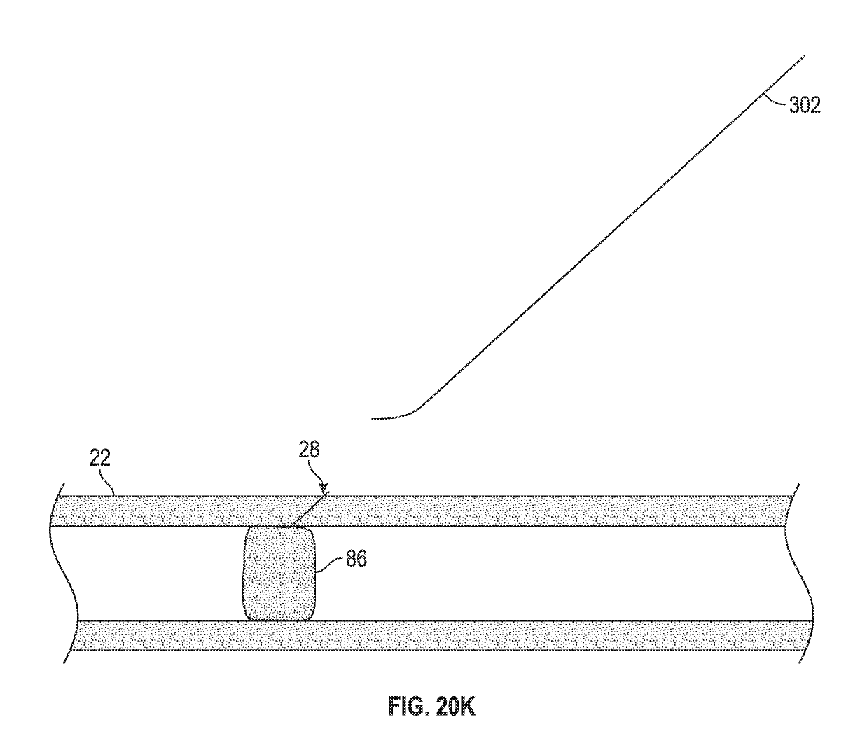

Referring to FIGS. 20A-20K, an embodiment similar to that described in reference to FIGS. 16A-16K is illustrated, the embodiment of FIGS. 20A-20k featuring use of a guiding member such as a guidewire to facilitate efficient return to the arteriotomy location in an over-the-wire configuration after closure, until such guiding member or wire is removed. Referring to FIGS. 20A and 20B, an assembly is inserted and positioned as in FIGS. 16A and 16B. Referring to FIG. 20C, a guiding member (302), such as a guidewire, is inserted through a lumen defined in the foot member or elongate deployment member, through the collapsed closure device (86), and out distally into the vascular lumen. In another embodiment, a distal portion guiding member (302) may be preadvanced to a location within the collapsed closure device (86) before any instrumentation is inserted through the arteriotomy (28) as in FIGS. 20A and 20B. With the guiding member in place, the deployment of the closure device (86, 88) is continued, as shown in FIGS. 20D-20J, which parallel the deployment steps of FIGS. 16D-16J (with the exception that a plug member 294 is not shown in the embodiment of FIG. 20J, and a guiding member 302 is present in each step). Referring to FIG. 20J, with the tether member clipped and the closure device expanded (88), the guiding member (302) may remain in place until a time that the surgeon decides it may be removed (as in FIG. 20K), which may occur well after hemostasis of the arteriotomy. Should the operating team need fast and efficient access to the location of the arteriotomy and the associated vessel lumen while the guiding member remains in place, an over-the-wire procedure may be utilized to take further instrumentation directly to the site.

Referring to FIG. 21, a process for utilizing configurations such as those featured in FIGS. 20A-20K is illustrated. As shown in FIG. 21, after transcutaneous access and arteriotomy are created (160), an introducer advanced (162), and in this embodiment, a deployment assembly featuring a catch member inserted (208), and repositioned relative to the introducer (166), a guiding member such as a guidewire may be introduced (304), after which the deployment assembly may be withdrawn with feedback from a bleed-back configuration, as described above in reference to FIG. 11 (167). The foot member may be utilized to reposition and/or reorient the collapsed closure device and catch member (296), followed by urging the collapsed closure device and catch member against the arteriotomy location (298), expansion of the closure device (172), controlled retraction of the catch member (300), retraction of associated tools—with exception of the guiding member, which may be left in place (306), and shortening of the free length of the tether member while leaving the arteriotomy provisionally closed over the guiding member. After the surgical team decides that a deployed guiding member is no longer warranted, the guiding member may be removed (310), preferably by gently tensioning/pulling it out through the arteriotomy.

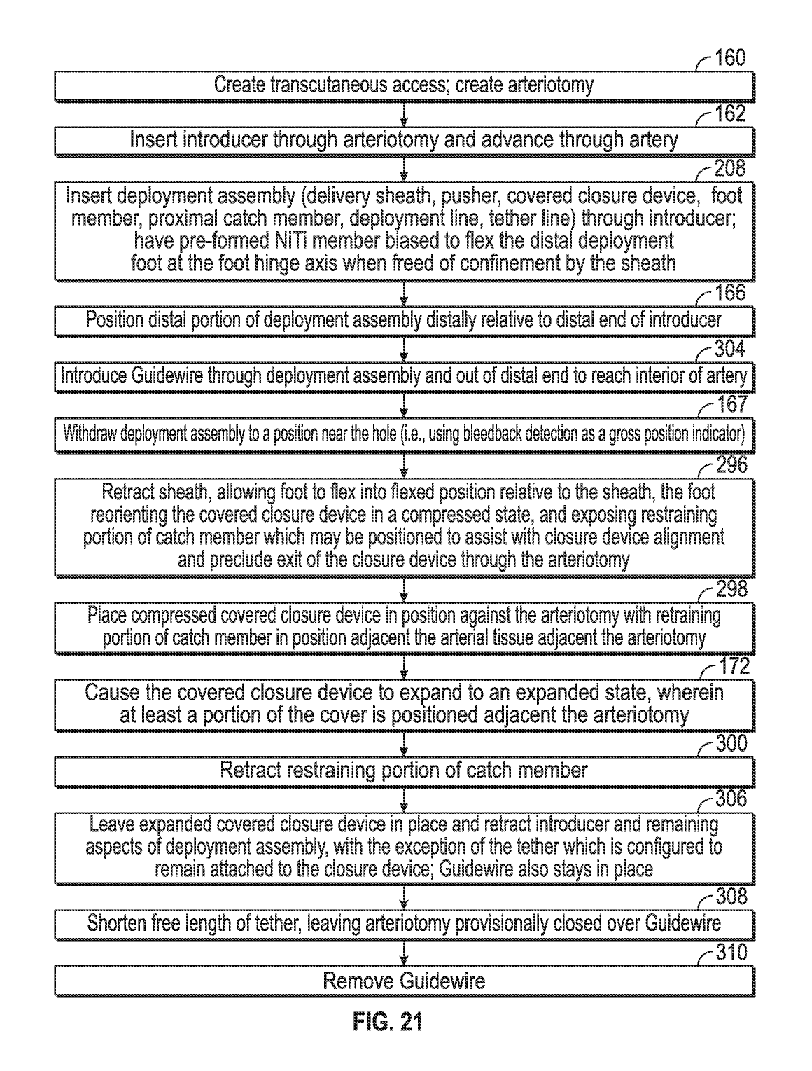

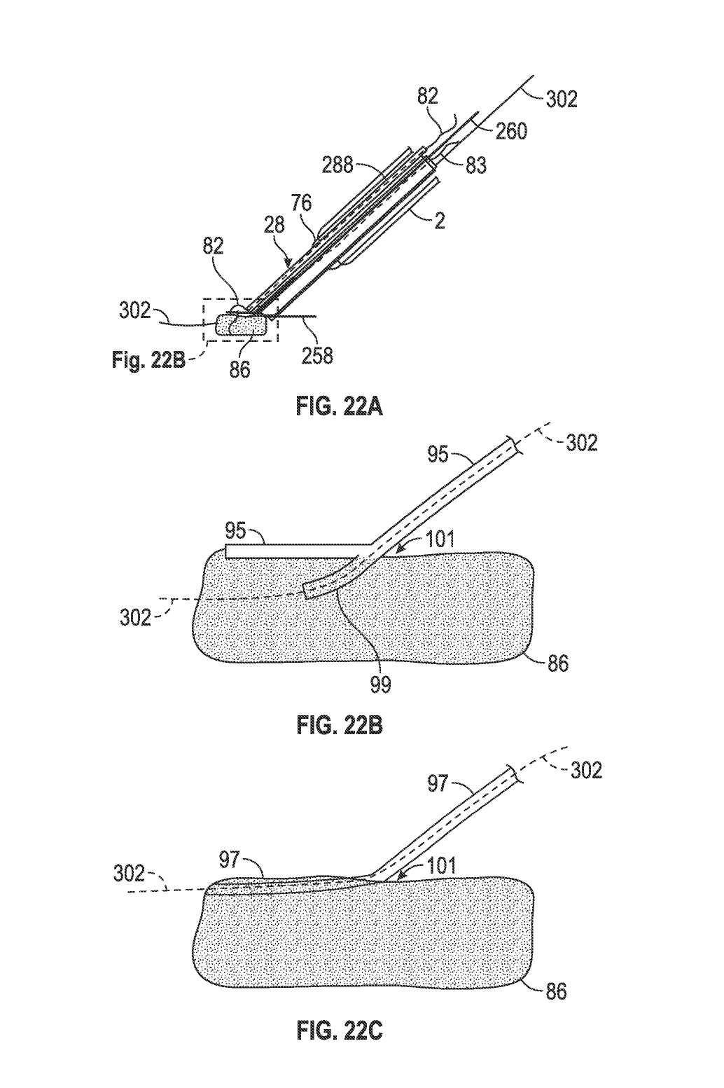

Referring to FIG. 22A, an embodiment similar to that depicted in FIG. 20G is depicted without the associated anatomy. FIG. 22B depicts a close-up view to illustrate that in one embodiment, the guiding member (302) may be passed through a lumen defined in part by a foot member portion (95), and in part by a conduit branch (99) configured to extend away from the foot member into the interior of the closure device (86), to direct the guiding member out into the vascular lumen without further entanglement. Referring to FIG. 22C, in another embodiment, the distal aspect of a foot member may be directed down into the closure device (86) to function both as a foot member for deployment purposes, and also as a guiding member conduit, without the need for a conduit branch (99—in FIG. 22B). In either variation, a small entry port (101) is created where the guiding member (302) exits the closure device (86), and this port (101) may be closed to facilitate hemostasis after removal of the guiding member by application of direct pressure, a small flap door that is biased to close, one or more sutures that may be controllably tensioned to close the port, or a plug member used similarly as described in reference to FIG. 16J.

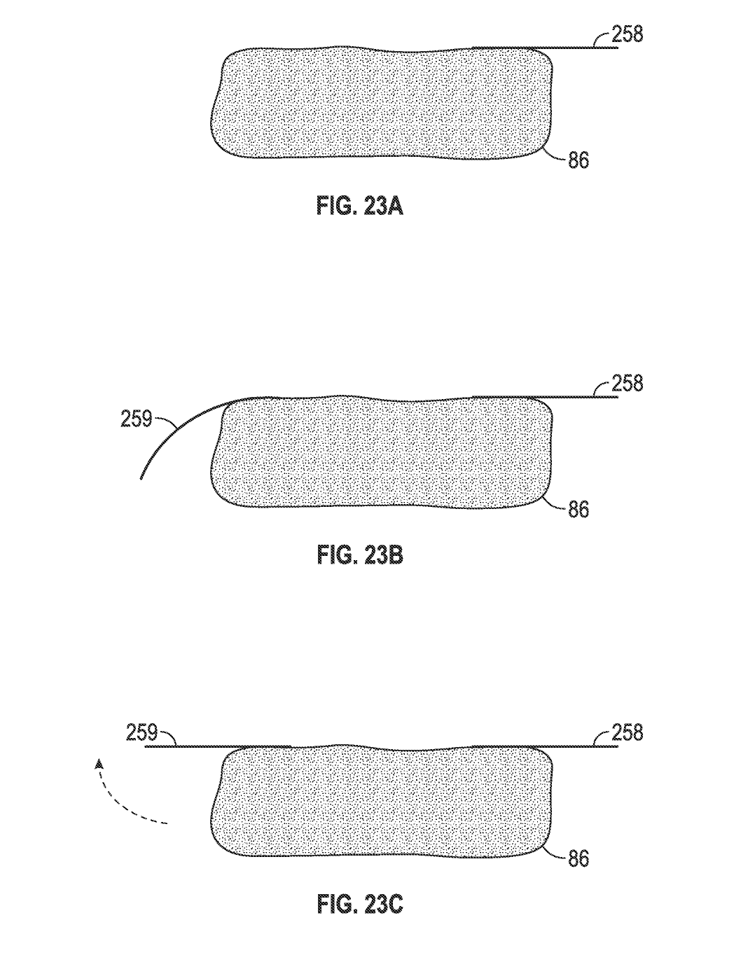

Referring to FIG. 23A, a simple illustration of a collapsed closure device (86) and associated catch member (258) is depicted. Referring to FIGS. 23B and 23C, in another embodiment, a distal catch member (259) comprising similar materials and being similarly controllably withdraw-able and/or repositionable, may be associated with the closure device (86) to prevent exit of the distal portion of the closure device back through the arteriotomy. The embodiment of FIG. 23C shows the distal catch member in a somewhat withdrawn configuration which may facilitate delivery and advancement through the arteriotomy. The embodiment of FIG. 23C shows the distal catch member in a fully extended position wherein it is configured to prevent withdrawal of the closure device (86) through an associated arteriotomy. The change from withdrawn to extended positions may be controllably executed by an operator pulling or pushing a push or pull member, such as a pushrod or tension element such as a wire or suture.

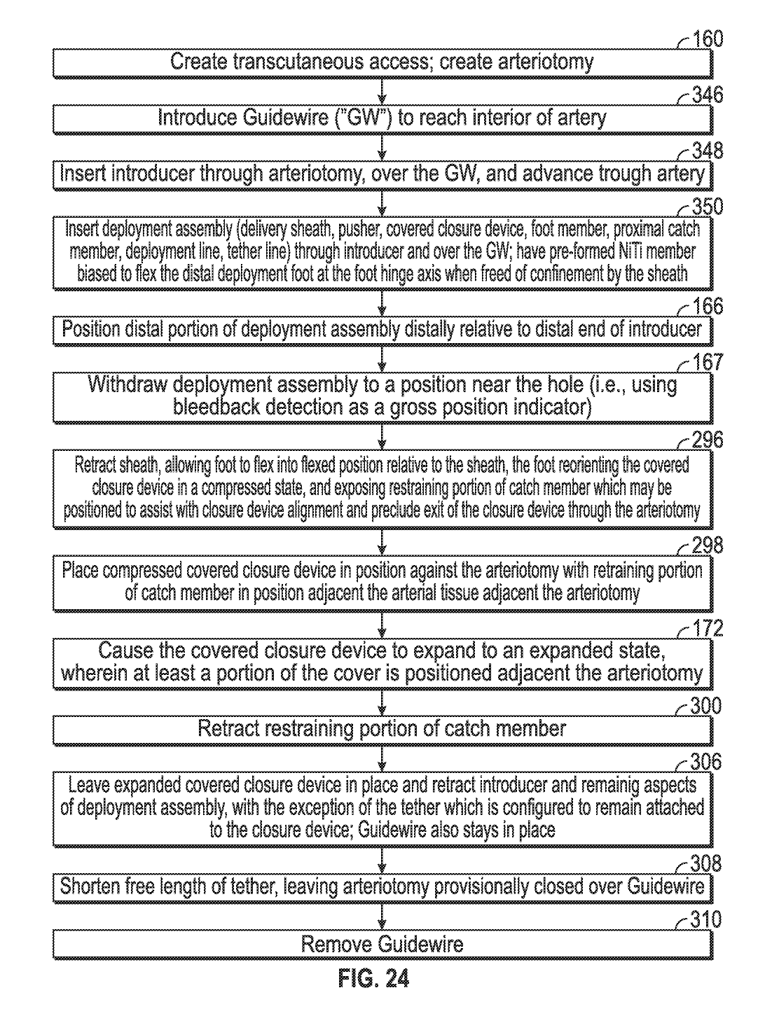

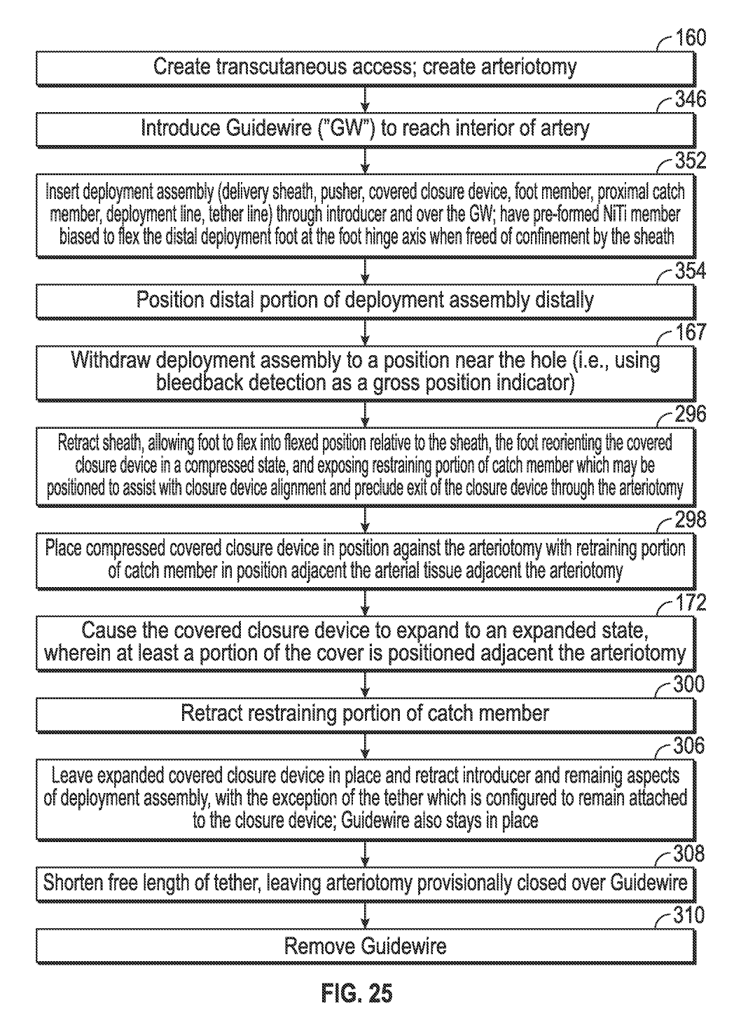

Referring to FIGS. 24 and 25, embodiments are illustrated wherein a guidewire is inserted before introduction of a deployment assembly, with other steps similar to those of other aforementioned embodiments, such as that of FIG. 21. Referring to FIG. 24, after creation of transcutaneous access and an arteriotomy, a guidewire (“GW”) may be introduced to reach the interior of the artery (346). Subsequently, an introducer may be advanced in an over-the-wire configuration (348), followed by a deployment assembly through the introducer and over-the-wire. Subsequent steps are similar to those described in reference to the illustrative embodiment of FIG. 21. Referring to FIG. 25, an embodiment similar to that of FIG. 24 is depicted, with the exception that the guidewire (“GW”) may be utilized to assist with positioning of the deployment assembly—without the use of an introducer. As shown in FIG. 25, after creation of transcutaneous access and an arteriotomy (160), the guidewire may be introduced (346), followed by the deployment assembly in an over-the-wire configuration, without an introducer (352). Subsequently the deployment assembly position may be adjusted, without the presence of an introducer (354), and the remaining steps may be similar to those described in reference to the illustrative embodiment of FIG. 21.

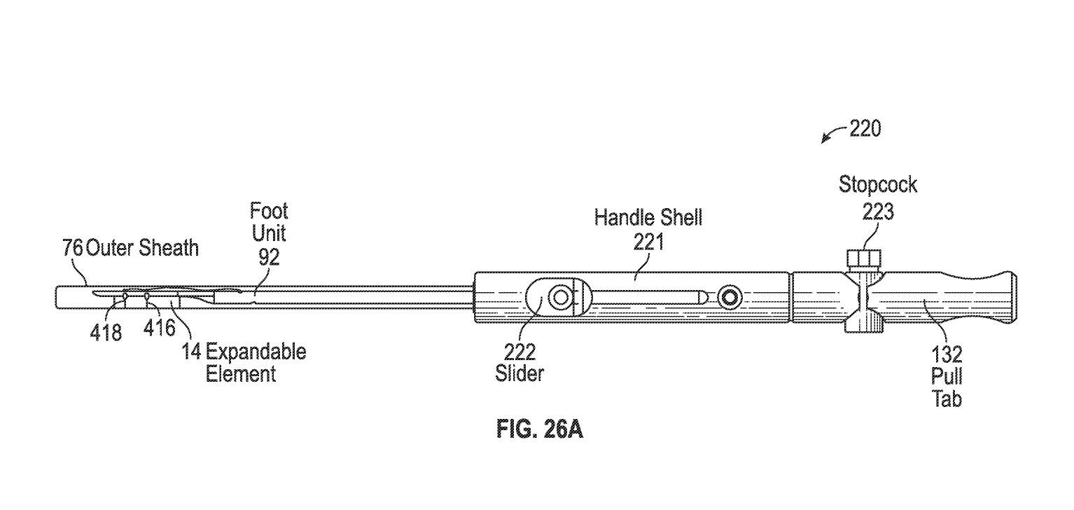

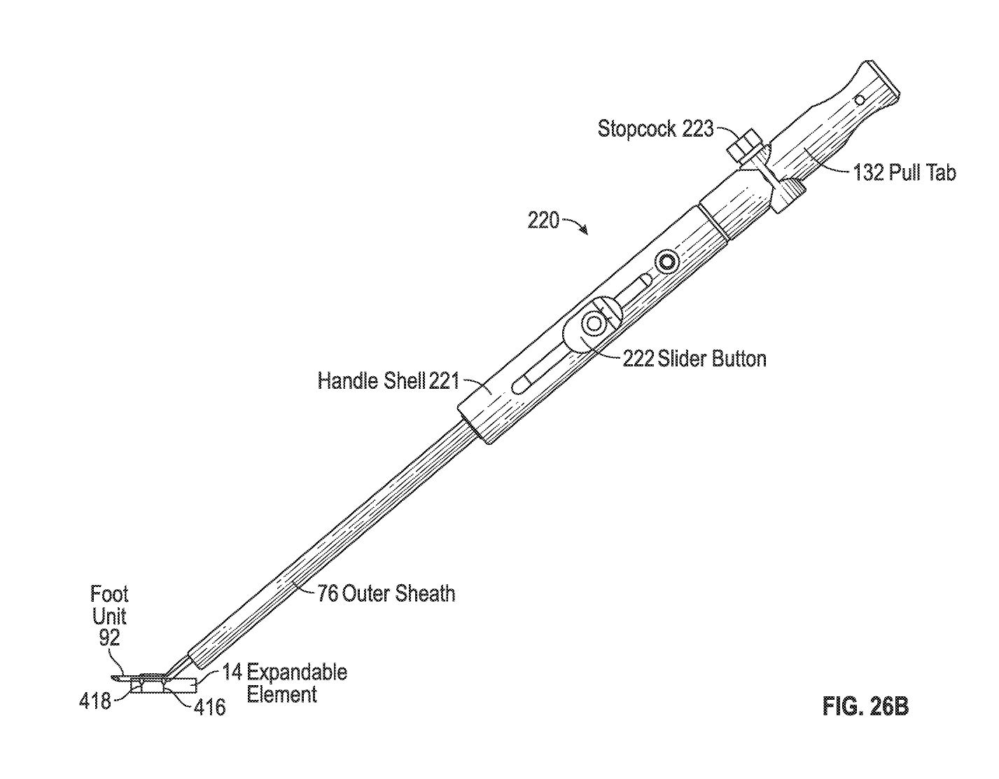

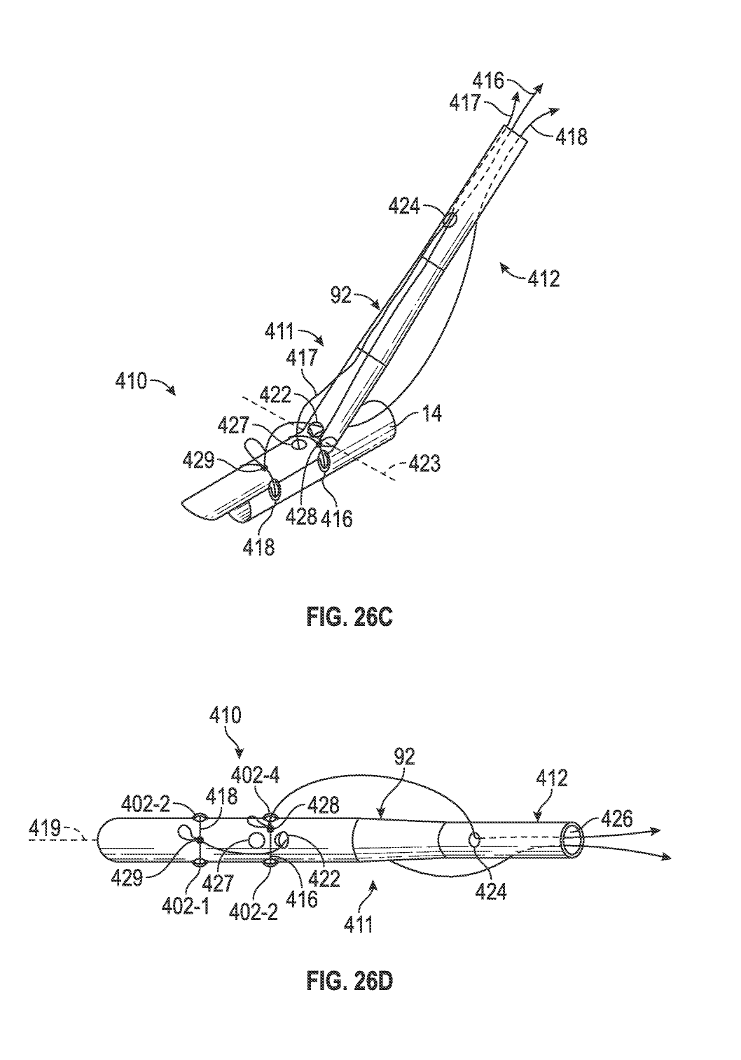

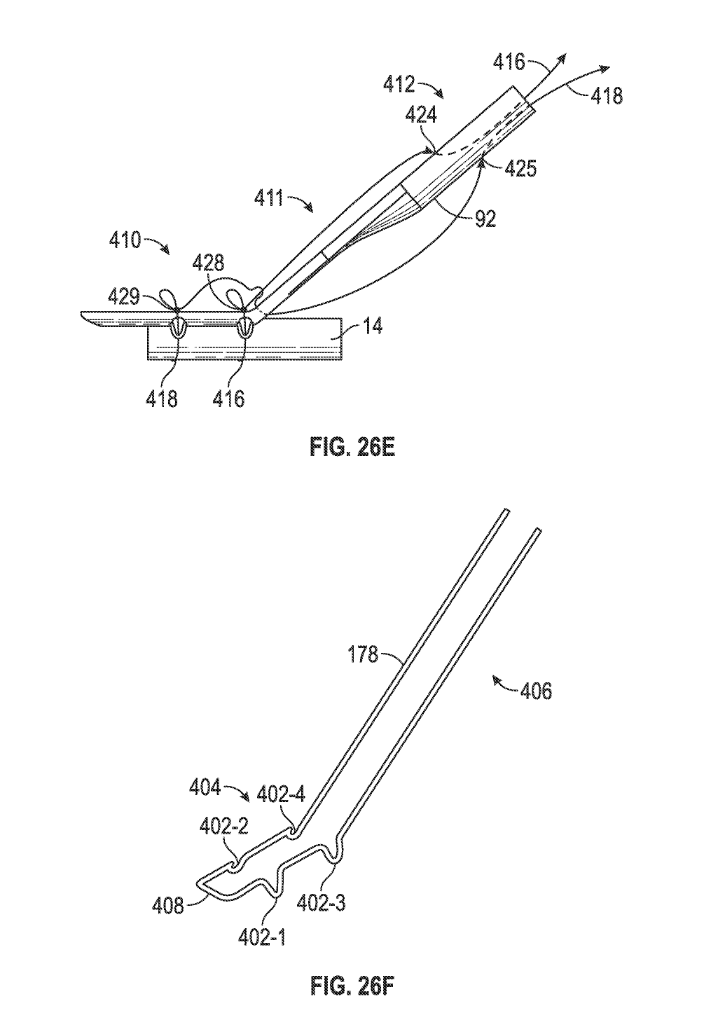

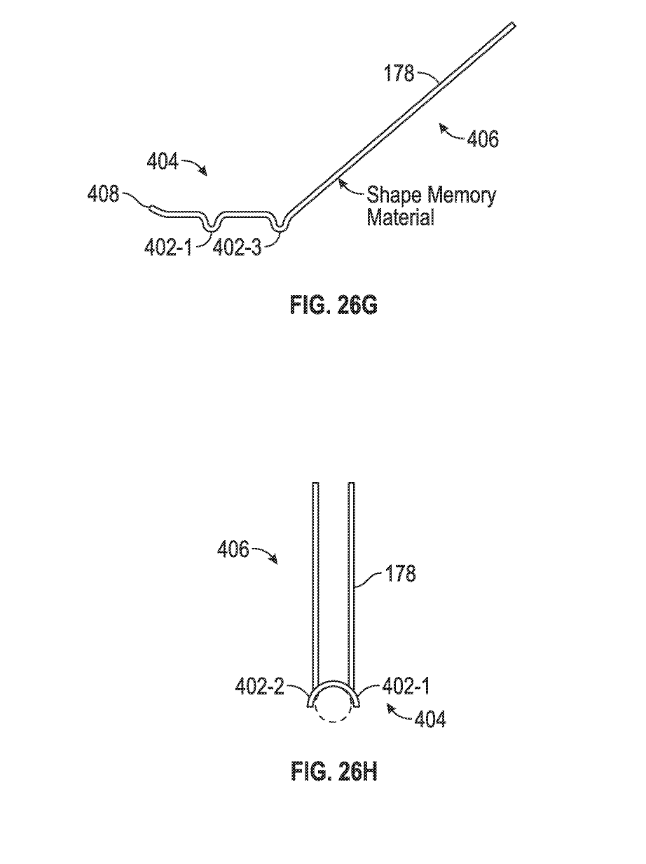

FIGS. 26A-H depict additional example embodiments of the delivery system. FIG. 26A is a side view of the delivery system where the components are housed within outer sheath (76) (shown in cross-section) prior to delivery of the closure device. The introducer is not shown. Outer sheath (76) is coupled with handle housing assembly (220) having a housing or shell (221) on which a sheath actuator (222) is disposed. A release actuator (132) is configured to release hitches (416, 418) simultaneously (although staggered release is also possible). A release safety mechanism (223) holds hitches (416, 418) and tether (417) (described with respect to FIGS. 26C-E) in tension and prevents actuation of release actuator (132) until the desired point in the procedure. Here, sheath actuator (222) is configured as a slidable handle, release actuator is configured as a pull tab, and release safety mechanism (223) is configured as a stopcock. Other alternatives for each will be readily apparent to those of ordinary skill in the art (e.g., levers, switches, dials, depressible buttons, etc.).

In this embodiment, foot member (92) is statically connected to handle (220) and thus not slidable with respect to handle (220). Conversely, outer sheath (76) is slidably coupled with handle (220) such that it is moveable with respect to foot member (92). (Either or both of outer sheath (76) and foot member (92) can be moveable or slidable with respect to each other and handle (220).) A seal (not shown), such as an o-ring and the like, can be placed between foot member (92) and outer sheath (76) to prevent blood flow proximally through the intervening space. The seal preferably provides a close fit while allowing sheath (76) to slide with respect to foot member (92).

FIG. 26B is a side view of the delivery system with outer sheath (76) proximally retracted with respect to foot member (92). In both FIGS. 26A and 26B outer sheath (76) is shown in cross-section so that foot member (92) and the other components of the delivery system are visible therein. FIG. 26C is a perspective view of foot member (92) with closure device (14) in the configuration deployed from within outer sheath (76), either through proximal retraction of outer sheath (76) with respect to foot member (92) or through distal advancement of foot member (92) with respect to outer sheath (76). FIG. 26D is a top-down view and FIG. 26E is a side view of the embodiment of FIG. 26C. In each of FIGS. 26C-E outer sheath (76) and the introducer are not shown for ease of illustration.

In this embodiment, foot member (92) is integrated with a foot frame that functions similarly to bending spring member (178). FIG. 26F is a perspective view, FIG. 26G is a side view, and FIG. 26H is a frontal view, each depicting foot frame (178) in the deflected state of FIGS. 26C-E. Foot frame (178) is preferably formed from a superelastic material, such as nitinol, and heat treated in the state of FIGS. 26F-H where a distal end portion (404) is deflected at an angle with respect to a proximal portion (406). Frame (178) is formed from a single continuous wire where the distal tip (408) of frame (178) is closed and the proximal end (409) is open, although each end can be closed or open and frame (178) can be formed from two or more discreet wire bodies. Distal portion (404) includes one or more partial loops or bends (402-1 through 402-4) that can be used to help retain tension members wrapped around the closure device (14).

Distal portion (404) of frame (178) is substantially encased within distal section (410) of foot member (92), which preferably has a concave lower surface for contacting or abutting the closure device (14). Proximal to section (410) is an intermediate section (411) that also has a concave lower surface for abutting or contacting the proximal portion of closure device (14) while within outer sheath (76) (prior to deflection). Intermediate section (411) transitions, or tapers, to a tubular proximal section (412) which is, in turn, connected or secured to the handle (see FIG. 26A).

In one example embodiment, foot member (92) is fabricated by molding a polymeric material (e.g., plastic, etc.) around foot frame (178) such that the two are integrated into one body. For instance, tubular plastic or polymeric stock can be placed into a mold with frame (178) and heated until a distal part of the tubular stock flows around and encases foot frame (178), leaving only the partial loops (402) exposed as depicted in FIG. 26E. In such a case, the portions of a tubular stock corresponding to sections (410) and (411) are heated until they flow (and melt) into the forms depicted in FIG. 26E. The tubular stock corresponding to proximal section (412) is not molded and retains its tubular shape with an inner lumen (426).

As can be seen in FIGS. 26C and 26E, partial loops (402-1 through 402-4) extend from the plastic or polymeric body to form eyelets bounded by the wire-body of foot frame (178) and the body of the plastic or polymer. Foot member (92) also includes three openings or apertures that can be located along the center line (419) of foot member (92) as seen in FIG. 26D. These apertures (424, 425, 426) (similar to apertures 204 and 206 of FIG. 9D) can be used to route tension members (also referred to as tethers, lines, or hitches) for controlling the placement and deployment of the closure device (14).

FIGS. 26C-E depict two hitches (416, 418) which are similar to the deployment tension members described herein. Hitch (416) preferably extends from the handle (not shown), along an inner lumen (426) of foot member (92), from inner lumen (426) through the sidewall of foot member (92) via aperture (424), distally along the upper surface of intermediate section (411) to a location just distal of the bend axis (423) of foot member (92). At this location, hitch (416) passes from the upper surface of deflectable distal section (410) downward through eyelet (402-3) around the outer surface of closure device (14) and back in an upward direction through eyelet (402-4) to the upper surface of distal section (410), where it is tied in a slip knot (428).

Hitch (418) passes from the hub, through inner lumen (419), from inner lumen (419) through the sidewall of foot member (92) by way of aperture (425) (located on the underside of the foot member), along the underside (or lower surface) of a portion of proximal section (412) and the majority of intermediate section (411), through aperture (422) in foot member (92) to the upper surface of distal section (410), and along the upper surface distally to a location at approximately the midpoint of the length of distal section (410). Here, hitch (418) extends downward through eyelet (402-1), around the outer surface of closure device (14), and back through eyelet (402-2) to the upper surface of distal section (410) where it is tied in a knot (429). Hitches (416) and (418) are preferably proximally retracted (i.e., pulled) to release knots (428) and (429), respectively, at the same time and allow closure device (14) to expand to its expanded state.

Referring to FIG. 26C, in addition to hitches 416 and 418, a third line (or tether) (417) is shown connected to the closure device (14) and passing along foot member (92). Unlike hitches (416, 418), tether (417) does not hold closure device (14) in the compressed state. Tether (417) is preferably coupled with a loop portion of the scaffold (similar to loops 121, 125, 127, etc. described above) and acts to hold the cover of closure device (14) to the underlying scaffold.

Tether (417) can take any desired route from the handle to closure device (14). In FIG. 26C, tether (417) extends from the handle (not shown), along the inner lumen of foot member (92), from the inner lumen through the sidewall of foot member (92) via aperture (424), distally along the upper surface of intermediate section (411) to a location just distal of the bend axis (423) of foot member (92). At this location, tether (417) passes from the upper surface of deflectable distal section (410) downward through aperture (427) and through the cover of closure device (14) where it then couples with the scaffold of closure device (14).

Alternatively, tether (417) could extend from the handle (not shown) through the inner lumen of foot member (92), from that inner lumen through the sidewall of foot member (92) by way of aperture (425), along the underside of a portion of proximal section (412) and the majority of intermediate section (411), through aperture (422) in foot member (92) to the upper surface of distal section (410), and along the upper surface distally to and through aperture (427), where it then couples with the scaffold of closure device (14) as described in the preceding paragraph. In yet another example (not shown), tether (417) could exit the inner lumen of foot member (92) via aperture (425) and proceed directly to closure device (14) without passing through the sidewall of foot member (92).

Referring back to FIG. 26H, it should be noted that partial loops (402-1 through 402-4) can be spaced apart from the collapsed closure device (shown in dashed line) in order to facilitate release of the knots and removal of hitches (416, 418).

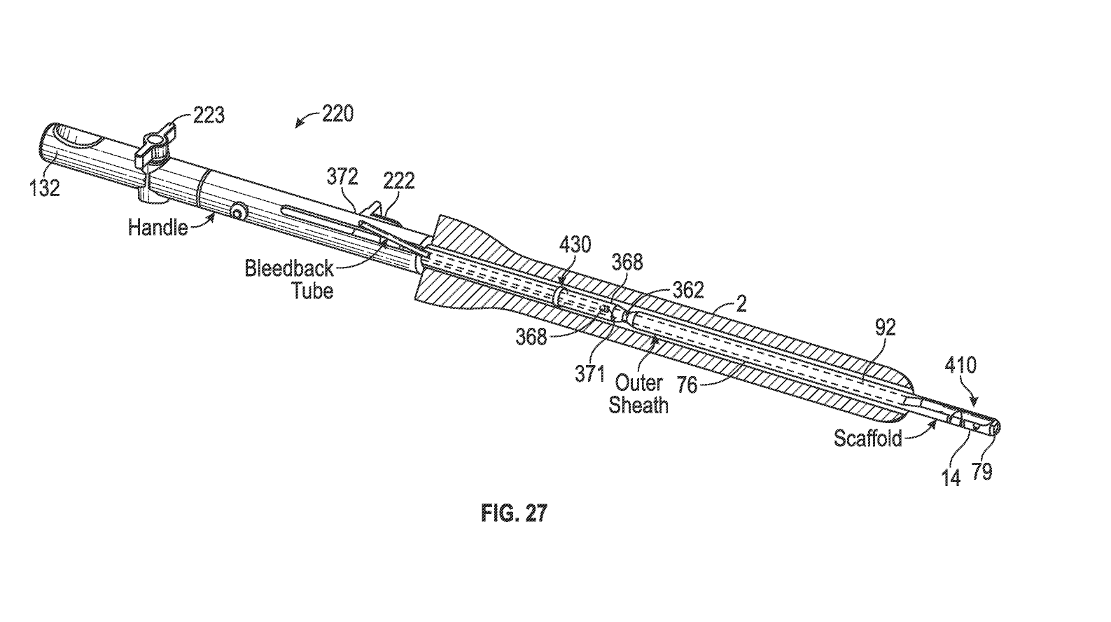

FIG. 27 is a perspective view of an embodiment of the delivery system described with respect to FIGS. 26A-H and the bleed-back subsystem described with respect to FIGS. 11D-E (inner tube (360) not shown). FIG. 27 depicts an introducer sheath (2) (transparent), with outer sheath (76) slidably located therein.

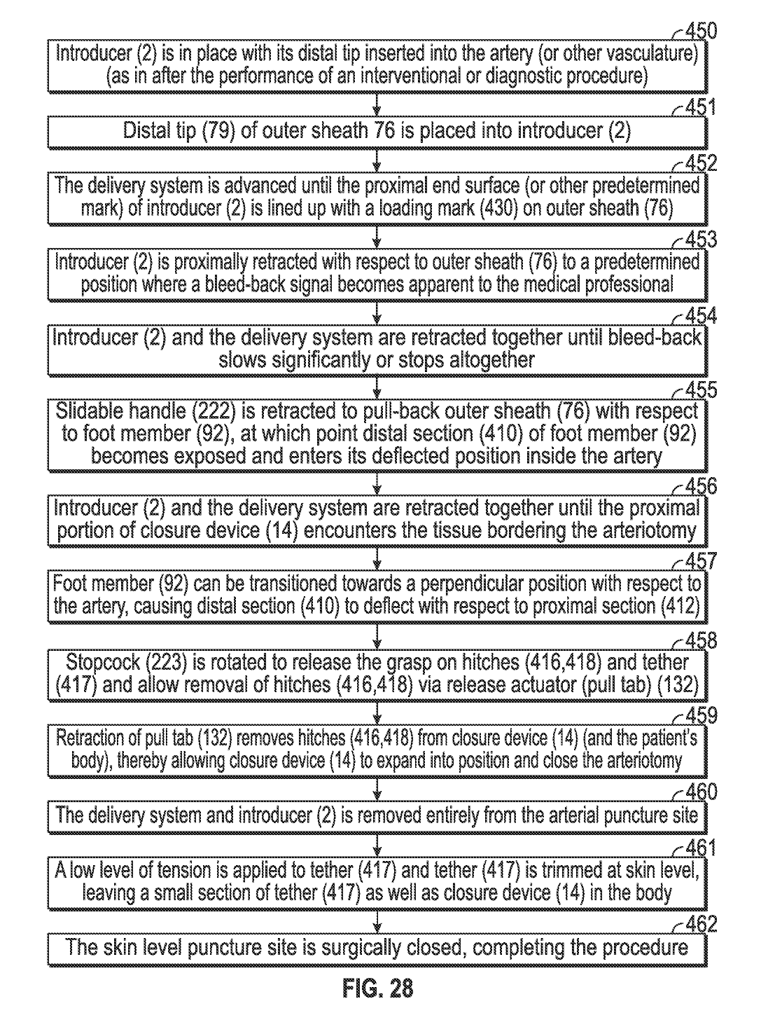

FIG. 28 depicts an example embodiment of an arteriotomy procedure, which is described with respect to the delivery system of FIG. 27. One of ordinary skill in the art will recognize that this example procedure can also be used in similar fashion with the other embodiments of the delivery system and bleed-back subsystem described herein.

Prior to use of the delivery system to deploy closure device (14), introducer (2) is preferably already in place with its distal tip inserted into the artery (or other vasculature) (as in after the performance of an interventional or diagnostic procedure) (see 450). Distal tip (79) of outer sheath 76 is then placed into introducer (2) (see 451). The delivery system is then advanced until the proximal end surface (or other predetermined mark) of introducer (2) is lined up with a loading mark (430) on outer sheath (76), at which point advancement of the delivery system with respect to introducer (2) is stopped (see 452). This position corresponds to the location of the outer sheath tip within the introducer but near the introducer (2) distal tip. The position could also be alignment of the outer sheath tip with the introducer distal tip (2).

Introducer (2) is then proximally retracted with respect to outer sheath (76) to a predetermined position, which, in this embodiment, is the contact of the proximal end surface of introducer (2) with the front distal-most surface of handle housing assembly (220) (see 453). Proximal retraction of introducer (2) is preferred over further advancement of the delivery system as it minimizes the risk of inadvertent pushing of the delivery system into the arterial wall. In this state, the outer sheath (76) preferably extends beyond the distal tip the introducer (2) by between about 10 mm and about 50 mm.

At this point the distal tip of introducer (2) is at a predetermined distance proximal to the distal tip of outer sheath (76) but also distal to bleed-back inlets (368). Preferably, blood is traveling proximally along the space between the inner diameter (ID) of introducer (2) and the outer diameter (OD) of outer sheath (76) to bleed-back inlets (368), where it enters into bleed-back channel (364). Blood continues proximally through bleed-back channel (364) vacating air (or other fluid) from that channel through the microvent located on the channel's proximal end. Blood also passes into bleed-back tube (370) and continues proximally through tube (370) and out of blood outlet (372) to provide a visual bleed-back signal to the medical professional.

Next, introducer (2) and the delivery system are retracted together until bleed-back slows significantly or stops altogether, which is indicative of the distal tip of introducer (2) exiting the artery (or arteriotomy) either partially or fully (see 454). The delivery system is now in the appropriate position for outer sheath retraction with the closure device located just inside the artery (in one example, by about 10 mm). Slidable handle (222) is then retracted to pull-back outer sheath (76) with respect to foot member (92), at which point distal section (410) of foot member (92) becomes exposed and enters its deflected position inside the artery (see 455). Introducer (2) and the delivery system are then refracted together until resistance is encountered (or felt by the medical professional), indicating that the proximal portion of closure device (14) has encountered the tissue bordering the arteriotomy (see 456). The assurance of anchoring and the resulting resistance can be enhanced by transitioning foot member (92) towards a perpendicular position with respect to the artery, e.g., by lifting or raising foot member (92), which causes distal section (410) to further deflect with respect to proximal section (412) (i.e., the angle of deflection increases between distal section (410) and the adjacent portion of foot member (92) on the opposite side of the bend axis) (see 457). In one example, distal section (410) transitions from a deflection angle of approximately 60 degrees to an angle of approximately 90 degrees.

Stopcock (223) can then be rotated to release the grasp on hitches (416, 418) and tether (417) and allow removal of hitches (416, 418) via release actuator (pull tab) (132) (see 458). Proximally retracting pull tab (132) likewise pulls hitches (416, 418) causing knots (428, 429) to be set free (or released). Further retraction of pull tab (132) removes hitches (416, 418) from closure device (14) (and the patient's body), thereby allowing closure device (14) to expand into position and close the arteriotomy (see 459). The delivery system and introducer (2) can then be removed entirely from the arterial puncture site (see 460). The attachment tether (417) remains connected to closure device (14) and extends out of the puncture site. A low level of tension is applied to the tether and the tether is trimmed at skin level, leaving a small section of tether and closure device (14) remaining in the body (see 461). Preferably one or more of the tether, closure device cover, and closure device frame are bio-absorbable and disintegrate within the body over time. The skin level puncture site is then surgically closed, completing the procedure (see 462).

As mentioned, the release of hitches (416, 418) can be staggered such that either hitch 416 or hitch 418 is released first, with the distal device hitch (418) preferably released first. Release of only one hitch can allow partial expansion of closure device (14) and, if the positioning of device (14) is not as desired (confirmed via imaging), then closure device (14) can be repositioned or removed with the exertion of relatively less force as compared to a fully expanded closure device (14). This staggered release can be accomplished either with two dedicated actuators (132) (i.e., one for each hitch), or with a single actuator having progressive release settings, where movement of the actuator a first amount causes a first hitch to be released and additional movement by a second amount causes the second hitch to be released (and so on for each hitch if there are more than two).

FIGS. 29A-B illustrate various aspects of an alternative embodiment of an arteriotomy closure system (520). Referring to FIG. 29A, the arteriotomy closure system (520) has a soft tip (532) wherein the soft tip (532) is configured to rotate with respect to the outer sheath (524). The closure system (520) may have a marker (526) at the proximal end of the outer sheath (524), an outer sheath hub (528) proximal to the marker (526), and a hook pull tab (530) that is coupled to the outer sheath hub (528). The closure system (520) is inserted and positioned in a manner similar to that described with respect to FIGS. 16A and 16B. In this embodiment, the arteriotomy closure system (520) may be inserted into a vascular sheath (not shown) that is already positioned in the vessel (through the arteriotomy). Preferably, the closure system is advanced into the vascular sheath until the distal end of the closure system (522) protrudes slightly past the distal end of the vascular sheath. Preferred placement of the outer sheath (524) with respect to the vascular sheath may be accomplished by use of the marker (526). Here, the length of the vascular sheath is known and the marker (526) is positioned such that alignment of the proximal end of the vascular sheath with the marker (526) results in the distal end of the outer sheath (524) extending a relatively small amount (preferably less than the typical diameter of the vessel) distal to the distal end of the vascular sheath (523).

In use, the surgeon may advance the outer sheath (524) within the vascular sheath (523) lumen until the marker (526) reaches a proximal portion (or the proximal end) of the vascular sheath. Thus, the outer sheath hub (528), the hook pull tab (530), and the proximal end of the outer sheath (524) may remain exposed once proper placement within the vascular sheath (523) is obtained.

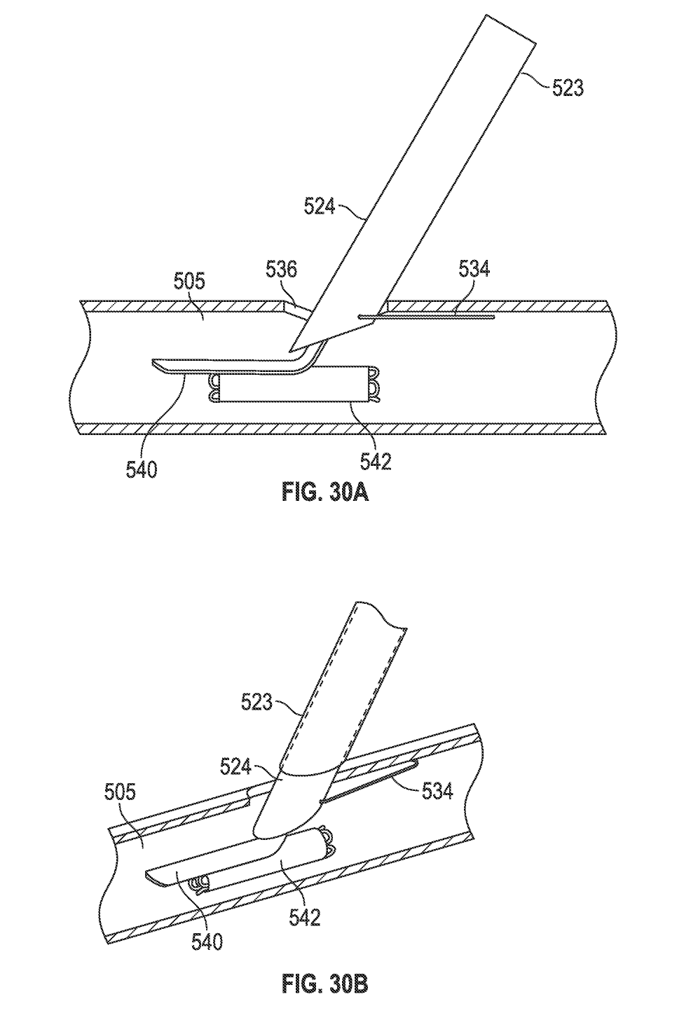

Referring to FIG. 29B, the distal portion (522) of the outer sheath (524) is illustrated. The distal portion (522) has a soft tip (522) and a hook (534), which is also be referred to herein as a catch member. The soft tip (522) may be a flexible, generally atraumatic polymer that is bendable and capable of sealing of the arteriotomy. The soft tip (532) may have a beveled edge such that the distal termination of the closure system (522) is diagonal. The soft tip (532) is oriented such that the longer side will be adjacent the arteriotomy during the procedure, and preferably impedes blood from flowing out of the arteriotomy during the closure procedure. The soft tip (532) may also have markers that are readily visible on an X-ray viewer during insertion. The soft tip (532) may have an anti-collapsing structure and may be shaped before use to perform an arteriotomy closure medical procedure. Please note that the soft tip can be used with all embodiments described herein and each hook embodiment can be freely used in every embodiment or interchanged with every other hook member in every embodiment. The hook (534), or catch member, may be used to prevent or otherwise hinder the inadvertent withdrawal of the outer sheath (524) prior to the proper deployment and placement of the closure device within the accessed artery (see FIGS. 30A-30B). FIGS. 30A-B illustrate a cross section and an elevated perspective view of an alternative embodiment of an arteriotomy closure system (520), wherein a hook (534) protrudes from the distal end of the outer sheath (524), in a proximal direction, to prevent inadvertent removal through the arteriotomy (536). Referring to FIG. 30A, upon contemporaneously inserting the outer sheath (524), coupled within the vascular sheath (523), as previously described, into the arteriotomy (536), the surgeon may advance the outer sheath (524) into the vessel (505) independent from the vascular sheath (523). This advancing step occurs while maintaining the vascular sheath in a constant position, thus, exposing the hook (534) within the vessel (505). Once the hook (534) is clear of the restraining vascular sheath, one may retract the outer sheath (524) sufficiently to cause the hook (534) to directly abut the vessel (505) from within the vessel (505) itself. With the hook (534) deployed, the outer sheath (524) is effectively prevented from being withdrawn from the vessel (505) unless and until the hook (534) is collapsed or otherwise removed from a position that impedes the withdrawal of the outer sheath (524).

Once the hook (534) is sufficiently caught on the inside vessel wall (505), the closure procedure may advance to deployment of the closure device itself. The foot member (540) may be advanced sufficiently so as to clear the outer sheath (524), causing the foot (540) to deflect or rotate into a position that places the foot (540) in substantial alignment with the length of the vessel (505). The scaffold and cover (542) (being removably coupled to the foot (540)) deflects contemporaneously with the foot member (540). The subsequent release and deployment of the scaffold and cover (542), i.e., the closure device, to occlude the arteriotomy has been described in detail in the foregoing portions of the specification but in this latter embodiment the foot (540) is moved proximally until it abuts the soft tip (532). That is, the soft tip (532) may be used as a backstop so that the user has a known and fixed object against which to pull the foot (540).

Referring to FIG. 30B, an elevated perspective view of an arteriotomy closure system (520) is shown, wherein a hook (534) protrudes from the distal end of the outer sheath (524), in a proximal direction, to prevent inadvertent removal through the arteriotomy (536).

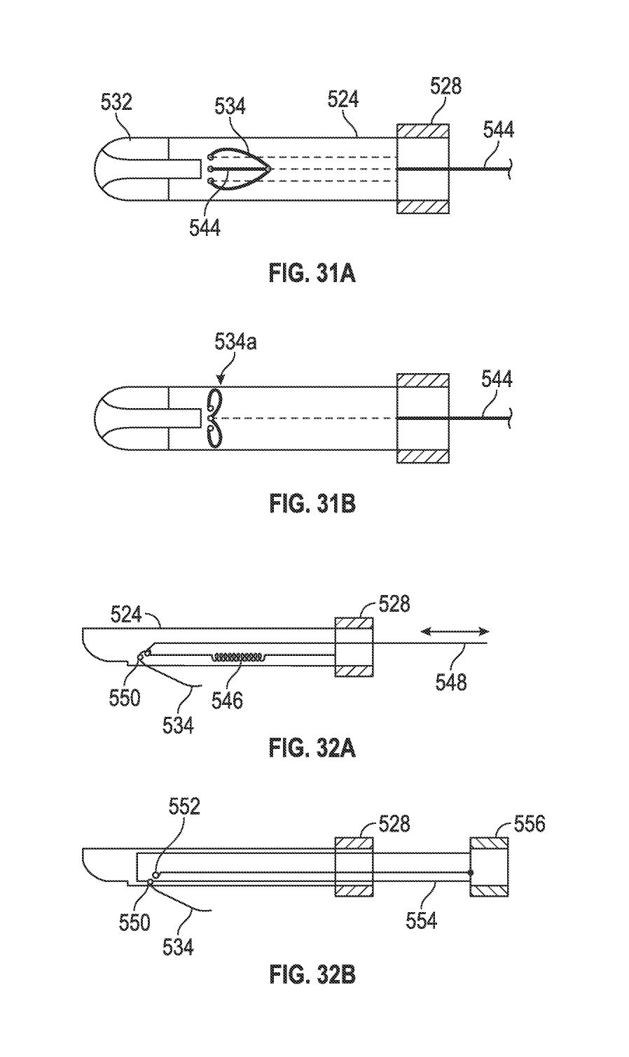

FIGS. 31A-31B illustrate a cross section of an alternative embodiment of an arteriotomy closure system (520), wherein a collapsible hook (534) protrudes from the distal end of the outer sheath (524), capable of being drawn towards the outer sheath (524) by manipulating a suture (544). Referring to FIG. 31A, an assembly similar to that of FIG. 9A is depicted, with the exception that a hook (534) is depicted extending proximally away from the proximal end of the collapsed closure device. In the depicted embodiment, the hook (534) is fashioned from one wire that is looped to form the hook (534), terminated at both ends to a hub (528). That is, both ends of the hook (534) are each bonded to the hub (528), the hook (534) extends into the sheath through two apertures (550) and each segment of the wire extend the length of the sheath. Accordingly, lateral manipulation of the hub (528) translates into lateral movement of the hook (534), as previously explained in connection with FIG. 13D. In instances where it is not desirable to have lateral movement of a hub (528), however, the hook (534) is nonetheless inwardly collapsible by exerting a pull force to the hook's (534) apex. This may be accomplished by using a suture (544) that is terminated at the hook's (534), e.g., at the apex, and routed into the inner lumen of the outer sheath (524) by way of an aperture that may be generally located in between the outer sheath (524) apertures that expose the hook. Referring to FIG. 31B, when the suture (544) is drawn proximally, the hook (534) deforms as the suture pulls the hook's (534) apex towards the outer sheath (524). With the hook (534) deformed, the hook (534) no longer sticks out, away from the outer sheath (524), thus, extraction from the hole or defect would be unrestricted by the previously extended hook (534) post-deployment of a closure device.

Hook (534) functionality may be obtained in a variety of example embodiments herein disclosed. For example, referring to FIGS. 32A-32B depicting alternative embodiments where the hook (534) is ultimately collapsed into the outer sheath (524), thus, allowing the unimpeded extraction of the deployment assembly from within the arteriotomy or hole. Referring to FIG. 32A, a hook (534) may be routed into the lumen of an outer sheath (524). The hook (534) may be attached to a rigid member (548) at its distal end. Due to its coupled nature, once the rigid member (548) is distally advanced, the hook (534) would slide distally through the aperture (550) and into the outer sheath (524). Since one termination of the wire forming the hook (534) is bonded to the hub (528), a spring (546) may be used to allow the distal and proximal movement of the rigid member (548).

Alternatively, referring to FIG. 32B, a hook (534) may be collapsed (or retracted) within the distal end of the inner lumen of the outer sheath (528) using an inner member hub (556) to apply a distal force to the hook (534). In this embodiment, a hook (534) is routed through a hole or aperture on the outer sheath (550) and through a hole or aperture in the inner member (554). The hook is routed proximally within the lumen of the inner member (554) and is coupled to the inner member (554), that is, the hook (534) may be bonded to a portion of the inner member hub (556). With the inner member (554) in a coaxial arrangement with the outer sheath (524), the two components are slidably coupled together, movable in the distal-proximal direction. To collapse the hook (534), the inner member (554) is slidably advanced in a distal direction, bringing the outer sheath hub (528) and the inner member hub (556) in close proximity of one another. This motion subsequently causes the hook (534) to get drawn within the lumen of the outer sheath (524).

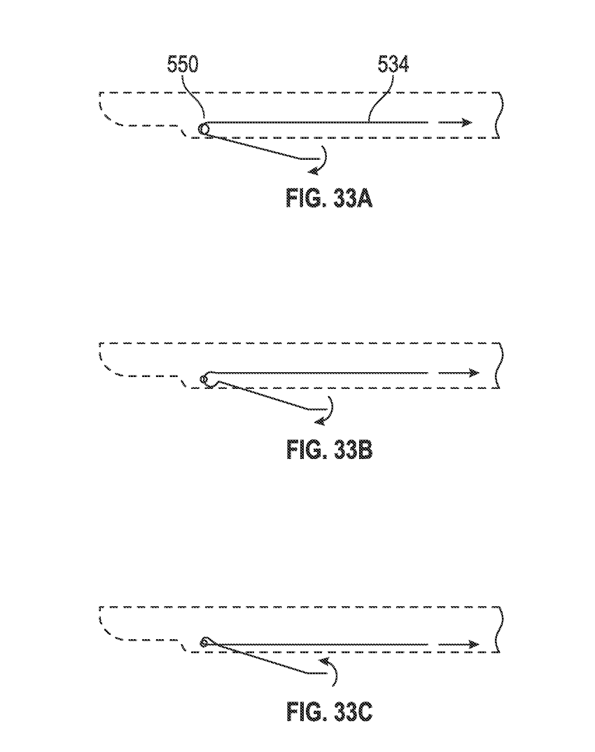

As may be readily appreciated by one of ordinary skill, it may be desirable to collapse the hook (534) using force directed in the proximal direction. FIGS. 33A-33C illustrate a cross section of an outer sheath (524), wherein a collapsible looped hook (534) protrudes from the distal end of the outer sheath (524) through exit holes or apertures (550). In this alternative embodiment, the hook (534) is retracted from its initial position by pulling on one end of the hook wires (534) in a proximal direction. The proximally directed force makes the hook (534) rotate about the hole (550) either towards or away from the outer sheath (524), dependent upon the shape of the bend.

Referring to FIG. 33A, a proximal force on the hook wires (534) may cause the hook (534) to pivot away from the outer sheath (524) at the hole (550). Depending upon the length of the exposed hook (534), this may not be desirable since the hook (534) may need to be retracted while the outer sheath (524) is in the blood vessel. Thus, the clockwise rotation of the hook (534) may cause the hook (534) to be driven toward the vessel wall opposite the arteriotomy, possibly shifting the closure device from the appropriate position or causing injury to the opposite vessel wall.

Referring to FIG. 33B, a “hairpin curve” is placed at the pivot point for the hook (534). In this embodiment, the clockwise rotation of the hook (534) is minimized by adding a hairpin curve to the pivot point, such as a sharply curved back, U-shaped turn. In response to a proximal force, the hook (534) may begin clockwise rotation, yet the added curve allows for the hook to return closer to the outer sheath (524) before complete removal is accomplished.

Referring to FIG. 33C, a greater than 360 deg. curve is placed at the pivot point for the hook (534). In this embodiment, the clockwise rotation of the hook (534) may be entirely eliminated. In response to a proximal force, and primarily due to the introduced greater than 360 deg. bend, the hook (534) may be drawn closer to the outer sheath (524), thereby minimizing risk of damage to the surrounding tissue within the vessel. Alternatively, a slot structure may be used in place of a hole or aperture (550). Having a slot that leads to the inner lumen of the outer sheath (524) may facilitate using a hook (534) that has a membrane surrounding the exposed hook (534). By using a membrane that substantially surrounds the hook (534), the membrane may keep the spacing in the hook (534) to remain relatively free of biological matter that may impede the hook's (534) ability to retract within the outer sheath (524). It should be noted that the membrane can be used with any embodiment of a looped hook or catch member described herein.

FIGS. 34A-34B illustrate a cross section of a closure device (564) configured to be deployed prior to the contemplated surgical procedure. FIG. 34A illustrates an example embodiment of a deployed closure device (564) within the vessel (562), the closure device (564) having a tether (561) extending therefrom through an arteriotomy (563). The closure device (564) may be deployed in accordance with the teachings of the present disclosure, but in this alternative embodiment, however, the deployment of the closure device (564) differs in that it occurs prior to performing the intended diagnostic or interventional surgical objective, e.g., heart valve repair, replacement, reconstruction, etc. For example, the arteriotomy opening (563) is first created and a guidewire (560) is inserted into the vasculature. The size of the opening (563) can be expanded by subsequent insertions of dilators over the guidewire (560) and into the opening (563), e.g., using the Seldinger technique. Once the opening (563) is large enough to receive any of the closure system embodiments of the present disclosure (e.g., outer sheath 524 of FIGS. 29A-B without the introducer), which is preferably smaller than the outer diameter of the introducer (2) for the interventional surgical device (e.g., heart valve replacement catheter), the closure system can be inserted into the arteriotomy and the closure device can be deployed in accordance with any of the embodiments disclosed herein. Once deployed, the tether (561) extends through the arteriotomy and may be used for anchoring the closure device (564) during the insertion or removal of other elongate surgical instruments so as to prevent the closure device (564) from slipping longitudinally along the vessel (562). The guidewire (560) will remain in place in the space between the vessel (562) wall and the closure device (564) as shown in FIG. 34A.

With the tether (561) anchored, a user may then enlarge the arteriotomy (563) even further to accommodate the (typically) larger introducer or vascular sheath (566). Considering that the deployed closure device (564) is collapsible, the insertion pressure of the vascular sheath (566) will collapse a portion of the closure device (564) at one end, as shown in FIG. 34B. The temporarily deformed closure device (564) preferably remains immobile in the vessel (562) while the vascular sheath (566) is used to introduce the diagnostic, therapeutic, or interventional instrument. To accommodate the introduction of a vascular sheath (566) into the arteriotomy (563) so as to partially collapse the closure device (564), the polymer cover that surrounds the scaffold to comprise the closure device (564) may have to be reinforced to prevent the vascular sheath (566) from damaging the cover. This may be accomplished by designing the top (superior) side of the polymer cover to be thicker than the bottom (inferior) side, thus, capable of withstanding aggressive insertion of the vascular sheath (566). Alternatively, the polymer cover can be designed to have multiple layers, including a safety layer (or shield) that is removably coupled to the polymer cover. The guidewire (560) may remain in coaxial arrangement with the vascular sheath (566) to assist in the distal routing of additional surgical tools and/or materials. Once the primary surgical objective has been completed, the vascular sheath (566) may be proximally removed until it clears the arteriotomy (563). As the vascular sheath (566) exits the arteriotomy (563), the closure device (564) returns to its original deployed form, as depicted in FIG. 34A. If a removable safety layer is used, this layer may be removed after withdrawal of the vascular sheath (566). As one of ordinary skill may appreciate, although this disclosure depicts a vascular sheath (566) being inserted to facilitate the collapse of the closure device (564), any similar lumen may perform the same function, for example, any hollow or solid elongate device that is of substantial enough diameter to pressure the closure device (564) to deform and allow clearance into the vessel (562). Deployment of the closure device (564) prior to performing the primary surgical procedure allows the closure process to occur when the arteriotomy opening (563) is smaller, i.e., before the arteriotomy opening (563) has been expanded to accommodate a large vascular sheath/introducer. This allows the closure procedure to occur with less potential for loss of blood by the patient, given the smaller opening (563).

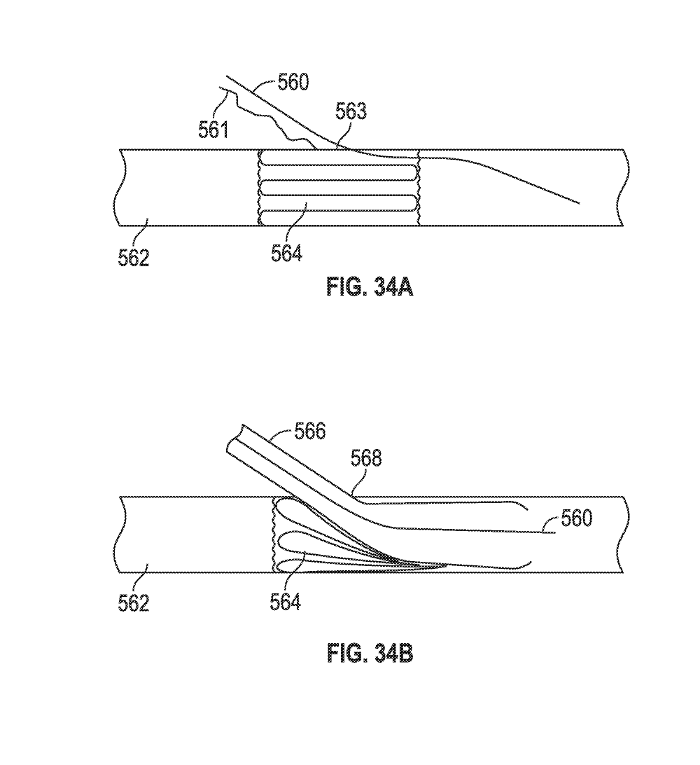

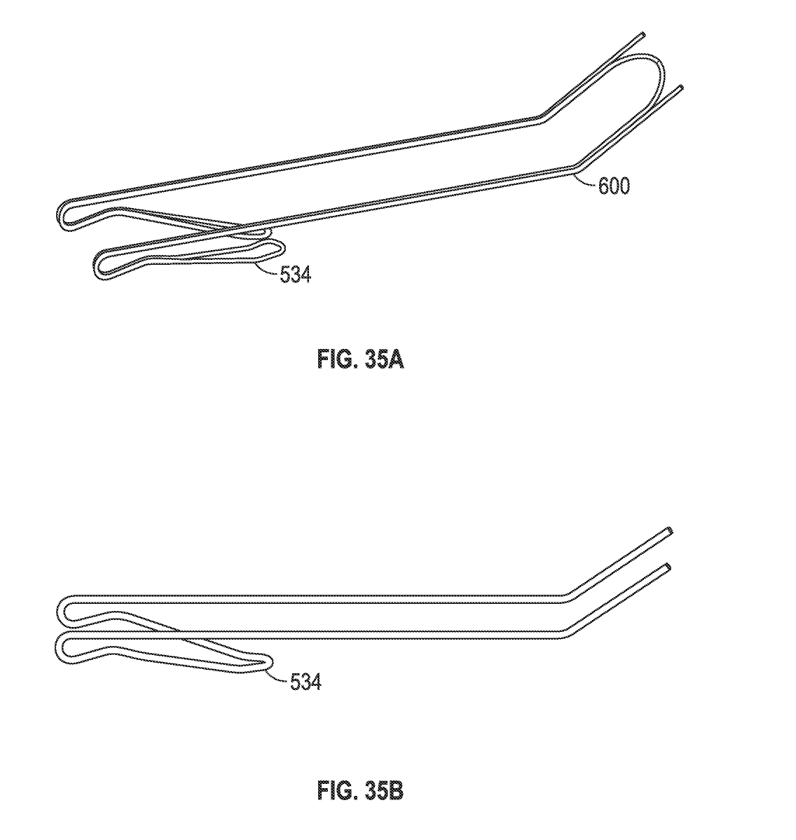

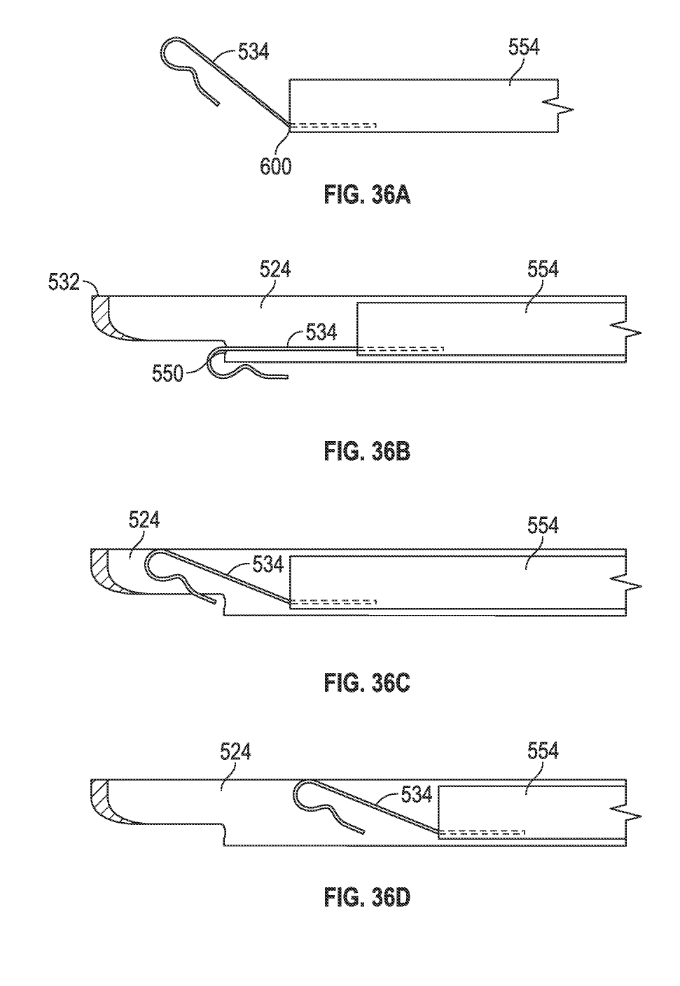

FIGS. 35A-35B illustrate example embodiments of a hook wire (534) for use in the example embodiments of FIGS. 36A-36D, 38A-38D, and 39A-39C. As detailed above with respect to FIGS. 29 and 30, the hook wire (534) is configured to engage on to an arteriotomy in a vessel. As depicted in FIG. 35A, the hook (534) may be have a bend (600) at a proximal point along the hook wire (534), causing the hook (534) to have a deflected state. The hook (534) is preferably formed from a superelastic material, such as nitinol (NiTi) or similar alloy. Hook (534) may be formed from a single continuous wire to form a single loop (FIG. 35B) or a double loop (FIG. 35A). In a single loop environment, the hook (534) may inadvertently catch onto tissue or other components of the vascular closure device like the scaffold, tether, or tensile elements. Therefore, it may be more desirable to use a double loop design wherein the individual wire legs can separate from each other and allow objects to pass through without departing from its intended purpose.

FIGS. 36A-36D illustrate a cross section of an alternative embodiment of an arteriotomy closure system (520), wherein a collapsible hook (534) is attached to a bottom portion of a distal end of an inner sheath (554) and protrudes through the outer sheath (524), and is capable of being drawn within the outer sheath (524) by manipulating the inner (554) and outer sheaths (524) in coaxial fashion. In this alternative embodiment, the hook (534) may be attached or otherwise bonded or affixed to a bottom portion of the distal end of the inner sheath (554), with the bend (600) aligned so that the hook (534) enters a new angle as it exits the inner sheath (554), as depicted in FIG. 36A. The inner sheath (554) is slidably inserted into an outer sheath (524), or other tubular polymer extension. After insertion in a distal direction, the hook (534) is routed through an exit hole (550), and retracted in the proximal direction so as to fully deploy the collapsible hook (534), ready to engage an arteriotomy and depicted in FIG. 36B. Once transvascular access is no longer desirable and the closure device has been deployed, the hook (534) may be disengaged from the arteriotomy. The inner sheath (554) is advanced distally with respect to the outer sheath (524), causing the hook (534) to enter the hole (550) and rotate about the bend (600), coming to rest within a hollow portion of the outer sheath (524) at its distal end (FIG. 36C). The bend (600) facilitates the rotation since the hook (600) is biased towards the upper area of the outer sheath (524). With the inner sheath (554) and the hook (534) both substantially housed within the lumen of the outer sheath (524), the inner sheath (554) may be removed from within the outer sheath (524) and the arteriotomy without interference of any object, as shown in FIG. 36D.

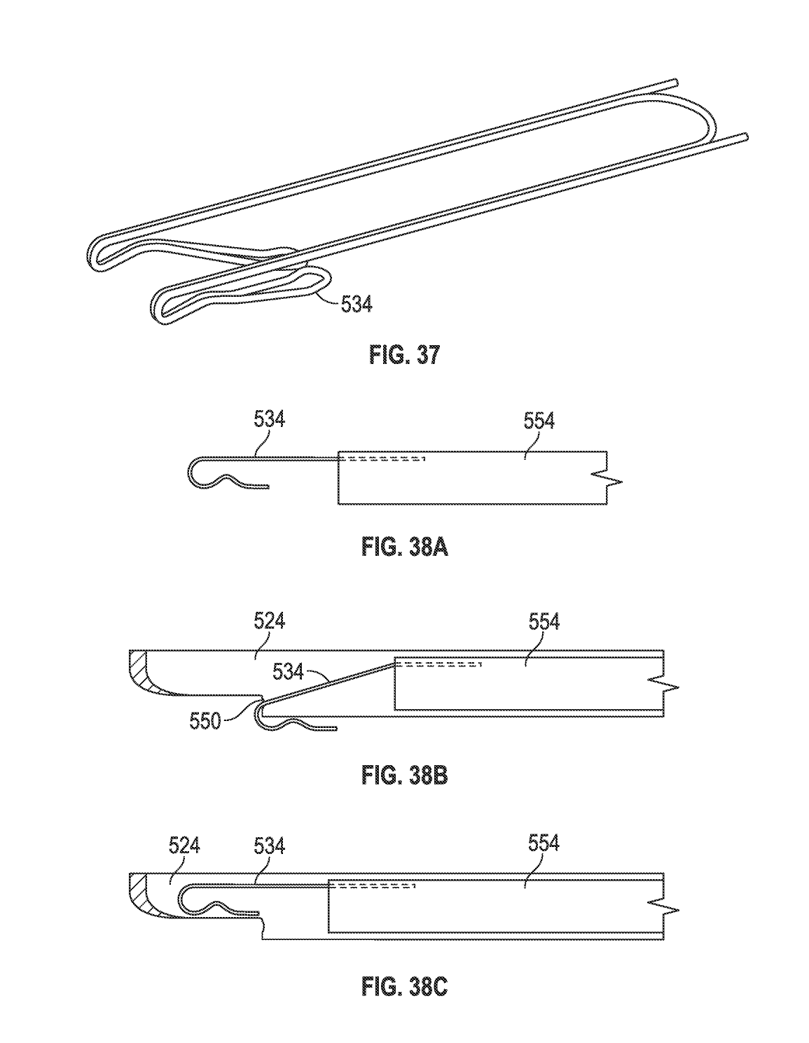

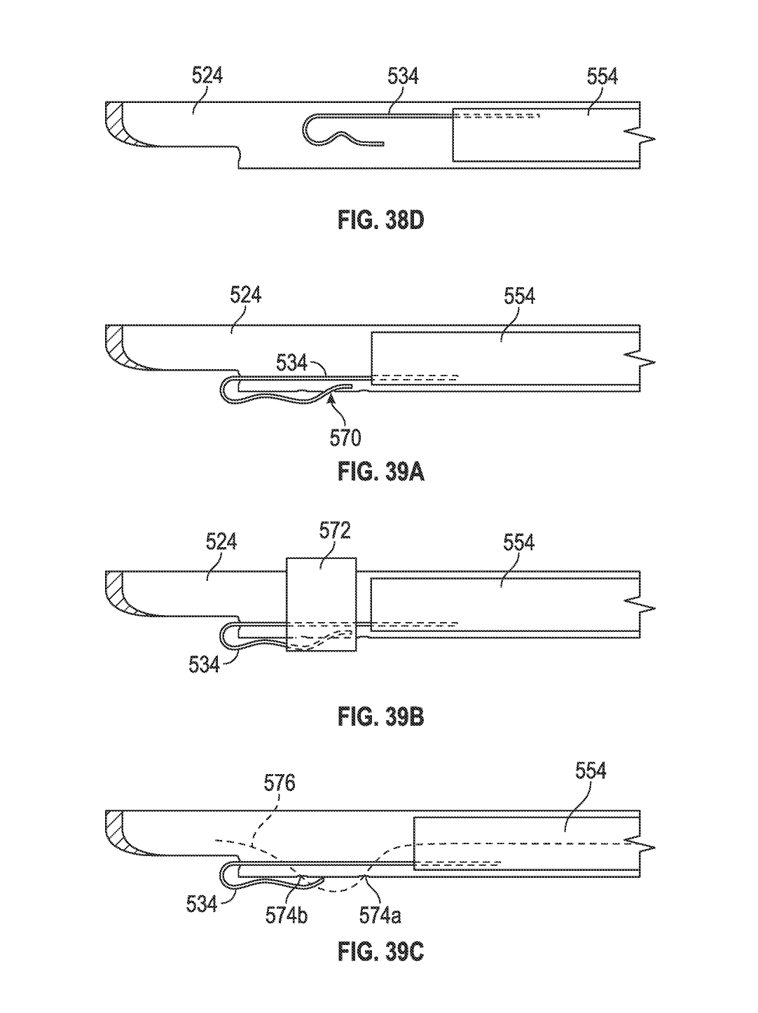

FIG. 37 illustrates an example embodiment of a hook wire (534) for use in the example embodiments of FIGS. 38A-38D. As detailed above with respect to FIGS. 35A and 35B, the hook wire (534) may alternatively be configured to not have a bend in connection with other embodiments but still be able to engage on to an arteriotomy in a vessel. Rather than to have a deflected state, pre-biased at a bend (600), the hook (534) may be shape-set straight but may otherwise be similar in all other regards, for example, formed from a superelastic material, formed from a single continuous wire to form a single loop or a double loop.

FIGS. 38A-38D illustrate a cross section of an alternative embodiment of an arteriotomy closure system, wherein a collapsible hook (534) is attached to a top portion of a distal end of an inner sheath (554) and protrudes through the outer sheath (524), through a hole (550), and is capable of being drawn within the outer sheath (524) by manipulating the inner (554) and outer sheaths (524) in coaxial fashion. In this alternative embodiment, the hook (534) may be attached or otherwise bonded or affixed to a top portion of the distal end of the inner sheath (554). The inner sheath (554) is slidably inserted into an outer sheath (524), or other tubular polymer extension, and the hook (534) is bent downwards and routed through the hole (550) of the outer sheath (524). Once the hook (534) is properly routed through the hole (550), the inner sheath (554) may be slightly retracted to fully deploy the hook (534). Once the underlying procedure is completed and the closure device is deployed, the hook (534) is disengaged from the arteriotomy. The inner sheath (554) is advanced distally with respect to the outer sheath (524), causing the hook (534) to enter the hole (550) and return to its shape-set straight formation, coming to rest within a hollow portion of the outer sheath (524) at its distal end (FIG. 38C). Since the hook (534) was bent to be routed through the hole (550), the hook (534) naturally returns to the upper area of the lumen of the outer sheath (524). With the inner sheath (554) and the hook (534) both substantially housed within the lumen of the outer sheath (524), the inner sheath (554) may be removed from within the outer sheath (524) and the arteriotomy without interference of any object, as shown in FIG. 38D.

As can be readily appreciated by one of ordinary skill, it is desirable to have the ability to collapse or otherwise confine the hook (534) until deployment of the hook (534) is warranted. Typically, the hook (534) is preferably placed in the collapsed state to facilitate arteriotomy insertion. Upon proper insertion and distal clearance of the arteriotomy itself, a user may deploy the hook (534) to prevent the inadvertent removal of the sheath. FIGS. 39A-39C illustrate cross sections of alternative embodiments of an arteriotomy closure system, wherein the collapsible hook is configured to be retained or otherwise compressed against the outer sheath (524) to facilitate subsequent deployment of the hook (534). FIG. 39A depicts an outer sheath (524) having a slit (570) on the underside of the outer sheath (524). In this embodiment, the hook (534) is routed through the hole (550) of the outer sheath (524) as disclosed in the various foregoing embodiments but, in this instance, the end of the exposed hook (534) is retained against the outer sheath (524) by temporarily inserting the end of the exposed hook (534) into the pre-formed slit on the exterior underside. The slit (570) thus retains the hook (534) until the hook (534), via the inner sheath (554), is moved distally so that the end of the hook (534) is released from the slit (570). Similarly, FIG. 39B depicts an alternative embodiment wherein a piece of polymer material is bonded to the outer surface of the outer sheath (524) to create a pocket (572) for the end of the hook (534). The hook (534) would be temporarily housed within the pocket (572) until the user deems it appropriate to deploy the hook (534) by advancing the inner sheath (554) distally until the hook (534) clears the pocket (572). FIG. 39C depicts another alternative embodiment wherein a wire (576) is used to retain the hook (534). A wire (576) is passed from the handle (not shown) and through a first small hole (574a) at the distal end of the outer sheath (524). The wire (576) is then routed over the hook (534) and into the outer sheath (524) through a second small hole (574b). In this latter example, the wire (576) is proximally drawn at the handle to free the hook (534) from its retained state, thus, deploying the hook (534).

It should be noted that each component described herein can be treated or coated to increase biocompatibility, promote a healing response or the formation of a thrombosis, or increase lubricity, to name a few. One with skill in the art will appreciate that one or more lubricious coatings (e.g., hydrophilic polymers such as polyvinylpyrrolidone-based compositions, fluoropolymers such as tetrafluoroethylene, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures.

Various exemplary embodiments are described herein. Reference is made to these examples in a non-limiting sense. They are provided to illustrate more broadly applicable aspects of the inventive subject matter. Various changes may be made to the embodiments described and equivalents may be substituted without departing from the true spirit and scope of the inventive subject matter. In addition, modifications may be made to these embodiments to adapt to the needs of each particular application, including alteration of materials, the alteration of a process step, the addition of a process step, the subtraction of a process step, the rearrangement of process steps, the alteration of a structural element or its function, the addition of a structural element, the subtraction of a structural element, and the rearrangement of components.

Further, as will be appreciated by those of ordinary skill in the art, each embodiment described herein is not to be viewed in isolation. Rather, each embodiment builds on the teachings and suggestions of each other embodiment. Each embodiment complements each other embodiment. Of course, the number of possible permutations of the embodiments of the vascular closure systems, devices, and methods described herein is so large that a description of each possible embodiment is not practical. Thus, it is intended that every element, component, feature, characteristic, function, and process step of every embodiment described herein is combinable with every other embodiment described here, unless clearly stated otherwise. Likewise, the mere recitation of an element, component, feature, characteristic, function, or process step in a particular embodiment does not mean it is required, rather, each element, component, feature, characteristic, function, or process step in a particular embodiment is intended to be optional unless clearly stated otherwise. Accordingly, each element, component, feature, characteristic, function, and process step described herein is claimable with every other element, component, feature, characteristic, function, and process step described herein within the bounds of logic of one of skill in the art.

Any of the devices described for carrying out the subject interventions may be provided in packaged combination for use in executing such interventions. These supply “kits” further may include instructions for use and be packaged in sterile trays or containers as commonly employed for such purposes.

The scope of this disclosure includes methods of using the subject systems, devices, and/or components thereof. The methods include the act of providing each system, device, and component described herein. Such provision may be performed by the end user. In other words, the “providing” act merely requires the end user obtain, access, approach, position, set-up, activate, power-up or otherwise act to provide the recited structure. Methods recited herein may be carried out in any order of the recited events which is logically possible, not only in that order recited.

Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any element, regardless of whether that element is stated as being “optional.” As such, this statement is intended to serve as antecedent basis for use of such exclusive terminology as “solely,” “only” and the like in connection with the recitation of claim elements, or use of a “negative” limitation.

Without the use of exclusive terminology, the terms “comprising,” “including,” and “having” in claims associated with this disclosure shall allow for the inclusion of any additional element—irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity. No claim element should be construed as invoking 35 U.S.C. section 112, paragraph six, without the explicit recitation of the language “means for” or “step for” in that claim element.

What is claimed is:

1. A delivery system for closing a hole in a vessel, comprising:
a deployable closure device;
an outer sheath having a distal end and a proximal end, wherein said distal end has a retractable hook; and
an elongate foot member having a deflectable foot coupled to the deployable closure device, wherein the outer sheath is in coaxial arrangement with respect to the elongate foot member, further wherein at least one tether couples the deployable closure device to the elongate foot member.

2. The delivery system of claim 1, further comprising a soft tip affixed to the distal end of the outer sheath.

3. The delivery system of claim 2 wherein the soft tip has a beveled distal termination.

4. The delivery system of claim 3 wherein the retractable hook is drawn towards the outer sheath as the outer sheath is coaxially advanced within a lumen.

5. The delivery system of claim 4 wherein the lumen comprises a vascular sheath.

6. The delivery system of claim 5 wherein the retractable hook moves away from the outer sheath once the retractable hook is no long restrained by the vascular sheath.

7. The delivery system of claim 6, further comprising a suture having proximal and distal ends, the distal end is bonded to the retractable hook and the proximal end is configured to be manually manipulated.

8. The delivery system of claim 7 wherein the retractable hook is retracted inwardly towards the outer sheath in response to the suture being pulled in a proximal direction.

9. The delivery system of claim 3 wherein the retractable hook is drawn into the distal end of the outer sheath.

10. The delivery system of claim 9 further comprising:
a spring configured to laterally expand and contract to facilitate the refraction and deployment of the retractable hook, wherein the retractable hook has a proximal end and a distal end;
a hub, wherein the proximal end of the retractable hook is bonded to the hub; and
a rigid member, wherein the rigid member is operably coupled to the hook at the distal end and extends proximally within the lumen defined by the outer sheath.

11. The delivery system of claim 9 further comprising:
an inner member in coaxial arrangement within the outer sheath, wherein a distal end of the inner member has a hole and a proximal end of the inner member has an inner member hub;
wherein the retractable hook, having a distal end external to the outer sheath, is routed through the outer sheath, through the hole at the distal end of the inner member, through the lumen defined by the inner member, and terminates an external end to the inner member hub; and
further wherein the inner member is configured to distally advance within the outer sheath lumen to draw the retractable hook into the distal end of the out sheath.

12. The delivery system of claim 9 wherein the retractable hook is configured to retract within the outer sheath in a proximal direction by exerting a proximal force on the proximal end of the retractable hook wires.

13. The delivery system of claim 12 wherein the retractable hook has a hairpin curve in close proximity to the holes on the outer sheath.

14. The delivery system of claim 12 wherein the retractable hook has a curve that is greater than 360 degrees in close proximity to the holes on the outer sheath.

15. The delivery system of claim 9 further comprising a membrane that substantially surrounds the distal portion of the retractable hook, wherein the retractable hook is configured to retract within a lumen defined by the outer sheath through at least one slot on the outer sheath.

16. The delivery system of claim 9 further comprising a plurality of tethers coupling the deployable closure device with the elongate foot member.

17. The delivery system of claim 16, further comprising:
a handle assembly having a sheath actuator configured to slide the outer sheath;
a release mechanism configured to release at least two of the plurality of tethers from the deployable closure device; and
a safety release mechanism for controlling whether the release actuator can be actuated.

18. The delivery system of claim 17, wherein the plurality of tethers includes a first tether passing through a cover of the deployable closure device, a second tether wrapped along an outer surface of the cover of the deployable closure device, and a third tether wrapped along the outer surface of the cover of the deployable closure device.

19. A delivery system adapted to deliver a deployable closure device to a hole in a vessel, comprising:
a deployable closure device, the deployable closure device further comprising a scaffold and a cover;
an outer sheath having a distal end and a proximal end, wherein said distal end has a retractable hook; and
an elongate foot member having a deflectable foot coupled to the deployable closure device, wherein the outer sheath is in coaxial arrangement with respect to the elongate foot member, further wherein at least one tether couples the deployable closure device to the elongate foot member.

20. The delivery system of claim 19 wherein the cover further comprises a two-layer, hermetically sealed, bioresorbable material having an inflation member extend therefrom.

21. The delivery system of claim 20 wherein the cover is configured to be inflated via the inflation member using a material selected from hydrogel, liquid, or gas.

22. The delivery system of claim 21 wherein the cover is configured to unfurl into an inflated state once an inflation material substantially fills the cover.

23. The delivery system of claim 22 wherein the inflated state of the cover is configured to occlude the hole upon reaching the inflated state.

24. The delivery system of claim 23 wherein the two-layers of the cover are spot welded together in an array.

25. The delivery system of claim 23 wherein the two-layers of the cover are slot welded together in an array.

26. The delivery system of claim 19 wherein the cover further comprises
a foldable bioresorbable material having a first and a second surface, the first surface having at least one aperture and the second surface having at least two apertures that are off-axis to the at least one aperture; and
a first suture routed through one of the at least two apertures on the second surface and out through the at least one aperture; and
a second suture routed through the second of the at least two apertures on the second surface and out through the at least one aperture.

27. The delivery system of claim 26 wherein the first and second surface are folded upon one another to partially sandwich the first and second sutures there between.

28. The delivery system of claim 27 wherein the cover in a collapsed state is furled for subsequent deployment within a vessel.

29. A method of closing an arteriotomy with a delivery system comprising an outer sheath, an elongate member having a deflectable foot, and a deployable closure device coupled with the deflectable foot, wherein the elongate member and deployable closure device are housed within the outer sheath, the method comprising:
distally advancing the delivery system into and through an inner lumen of an introducer to a first predetermined position;
proximally retracting the introducer with respect to the delivery system to a second position where a distal tip of the introducer is proximal to a distal tip of the outer sheath;
proximally retracting the introducer and the delivery system until bleed-back from the bleed-back port is at least significantly reduced;
proximally retracting the outer sheath with respect to the elongate foot member to allow a hook to deflect;
proximally retracting the outer sheath with respect to the elongate foot member to allow the deflectable foot to deflect with the deployable closure device;
proximally retracting the delivery system until the deployable closure device and hook are contacting the arteriotomy; and
deploying the deployable closure device.

30. The method of claim 29, wherein the elongate member is located within an inner tube and the inner tube is located within the outer sheath, and wherein bleed-back occurs through a space between the outer sheath and the inner tube.

31. The method of claim 30, wherein the bleed-back port is a proximal end of a bleed-back tube, the bleed-back tube having an open distal end located within the space between the outer sheath and the inner tube.

32. The method of claim 31, wherein the deployable closure device is deployed by:
transitioning a release safety mechanism from a position that prevents release of the deployable closure device to a position that permits release of the deployable closure device; and
actuating a release actuator to remove at least one tether coupling the deployable closure device to the deflectable foot such that the deployable closure device transitions from a contracted state to an expanded state.

33. The method of claim 32, wherein, in the second position, the distal tip of the introducer is distal to the at least one blood inlet.

34. The method of claim 32, wherein, in the second position, the distal tip of the introducer is proximal to the at least one blood inlet.

35. The method of claim 32, wherein the deployable closure device is held to the elongate member by a first hitch and a second hitch, and wherein releasing the deployable closure device comprises:
releasing the first hitch to allow a first portion of the deployable closure device to expand.

36. The method of claim 35, wherein releasing the deployable closure device further comprises:
repositioning the partially expanded deployable closure device; and
releasing the second hitch to allow a second, remaining portion of the deployable closure device to expand.

37. A method of treating a patient, comprising:
creating an opening in a blood vessel;
inserting a closure device delivery system through the opening and into the blood vessel;
deploying a compressible closure device from the closure device delivery system and into the blood vessel such that the closure device can close the opening;
removing the closure device delivery system from the body of the patient while leaving the closure device within the blood vessel;
inserting an elongate medical device through the opening and into the blood vessel, wherein insertion of the elongate medical device compresses the closure device away from the opening; and
removing the elongate medical device from the blood vessel, wherein removal of the elongate medical device allows the closure device to self-expand to a state that closes the opening.

38. The method of claim 37, further comprising inserting a guidewire into the blood vessel through the opening.

39. The method of claim 38, wherein the closure device delivery system is inserted over the guidewire, through the opening and into the blood vessel.

40. The method of claim 39, wherein the guidewire remains within the blood vessel when the closure device delivery system is removed from the body of the patient while leaving the closure device within the blood vessel.

41. The method of claim 39, wherein the elongate medical device is inserted over the guidewire, through the opening, and into the blood vessel.

42. The method of claim 37, further comprising removing a shield from the closure device after removing the elongate medical device from the blood vessel.

43. The method of claim 37, wherein the elongate medical device is an introducer or vascular sheath.

44. The method of claim 43, further comprising performing a diagnostic, therapeutic, or interventional procedure after the compressible closure device has been deployed and before the elongate medical device has been removed.

45. The method of claim 43, further comprising performing a valvuloplasty or valve replacement procedure after the compressible closure device has been deployed and before the elongate medical device has been removed.

46. The method of claim 43, further comprising inserting a intravascularly deliverable heart valve through the introducer or vascular sheath after insertion of the introducer or vascular sheath through the opening and into the blood vessel and prior to removal of the introducer or vascular sheath from the blood vessel.

47. The method of claim 37, further comprising enlarging the opening after deploying the closure device and before inserting the elongate medical device.

48. A delivery system for closing a hole in a vessel, comprising:
a deployable closure device;
an inner sheath having a distal end and a proximal end, wherein said distal end has a hook extending therefrom; and
an elongate foot member having a deflectable foot coupled to the deployable closure device, wherein the inner sheath is in coaxial arrangement with respect to the elongate foot member, further wherein at least one tether couples the deployable closure device to the elongate foot member.
